(19) United States
(12) Patent Application Publication
(10) Pub. No.: US 2026/0241232 A1
(43) Pub. Date: Aug. 20, 2026

(54) System And Method for Deterministic Generation and Adaptive Management of Personalized Pelvic Floor Physical Therapy Care Plan

(71) Applicant: Expect Fitness, Inc., New York, NY (US)

(72) Inventors: Dara Cook, New York, NY (US); Nicole Dugan, Philadelphia, PA (US)

(73) Assignee: Dara Cook, New York, NY (US)

(21) Appl. No.: 19/661,717

(22) Filed: Apr. 29, 2026

(51) Int. Cl. A63B 24/00; A63B 23/20

(52) U.S. Cl. CPC A63B 24/0075; A63B 23/20; A63B 2024/0068

(57) ABSTRACT

The present invention relates to a system and method for deterministic generation and adaptive management of a personalized pelvic floor physical therapy care plan. The system comprises an input module, a deterministic scoring module, an assignment module, a risk determination module, a care plan generation module, an outcome collection module, and a feedback module. The input module includes receiving patient-specific clinical data, including responses to a plurality of validated clinical assessment instruments. The deterministic scoring module computes one or more baseline clinical scores and one or more clinical flags from patient-specific clinical data using deterministic scoring functions. The assignment module assigns the patient to at least one exercise tier, at least one therapy profile, and one or more diagnosis codes based on baseline clinical scores and clinical flags. The risk determination module includes determining a patient's risk classification based on the exercise tier, therapy profile, diagnosis codes, baseline clinical scores, and clinical flags. The care plan generation module includes generating an individualized pelvic floor physical therapy care plan based on the patient's risk classification. The outcome collection module collects longitudinal treatment outcome data generated by the execution of the individualized care plan. The feedback module updates the baseline clinical scores, clinical flags, exercise tier, therapy profile, diagnosis codes, and patient risk classification based on longitudinal treatment outcome data for subsequent care plan generation. Therefore, the system and method enable automated, consistent, and adaptive generation of personalized pelvic floor therapy plans using deterministic clinical logic while continuously improving outcomes through feedback and data-driven insights.

### FIELD OF THE INVENTION

The present invention relates to the field of digital health systems and clinical decision support technologies, and more particularly to a system and method for deterministic generation and adaptive management of a personalized pelvic floor physical therapy care plan using patient-specific clinical assessment data and deterministic clinical logic. This improves consistency and transparency in therapy planning, enhances patient-specific treatment adaptation, and enables continuous refinement of care through data-driven insights and outcome-based feedback mechanisms.

### BACKGROUND

### Interpretation Considerations

This section describes the field of the invention in detail and discusses problems encountered therein. Therefore, statements in the section are not to be construed as prior art.

### Discussion

Pelvic floor physical therapy (PFPT) is a conservative, non-invasive treatment approach widely used for managing pelvic floor dysfunctions, including urinary incontinence, fecal incontinence, pelvic organ prolapse, chronic pelvic pain, and sexual dysfunction. These conditions affect a substantial portion of the population, with epidemiological data indicating that approximately one in four women in the United States experience a pelvic floor disorder. PFPT commonly includes pelvic floor muscle training, behavioral therapy, biofeedback, and adjunctive rehabilitation techniques designed to improve strength, coordination, endurance, and relaxation of pelvic musculature.

Conventional PFPT delivery relies heavily on clinician-guided exercise prescription and patient education performed during in-person visits. Although pelvic floor muscle training is widely recognized as a first-line intervention for multiple pelvic floor disorders, access to specialized pelvic health providers remains limited. Many patients experience extended waiting times for evaluation and treatment, particularly in rural or underserved regions. Additionally, social stigma, embarrassment, and lack of awareness further contribute to underdiagnosis and delayed treatment.

To address accessibility challenges, various digital health solutions have been developed to support PFPT outside traditional clinical environments. These systems include biofeedback devices, mobile applications, and sensor-based rehabilitation tools that guide exercise routines or monitor pelvic floor activity. Many digital platforms incorporate patient-reported outcome measures using validated clinical questionnaires to assess baseline symptoms and track progress over time.

However, existing PFPT systems lack standardized mechanisms for translating patient-specific clinical data into structured treatment protocols. The exercise prescriptions, dosage parameters, progression criteria, and adjunct therapy recommendations frequently depend on individual clinician expertise, leading to variability in care delivery. The questionnaire responses are often used only for classification or outcome monitoring rather than as structured inputs for automated care plan generation.

Digital PFPT systems also integrate multiple clinical indicators, such as symptom severity scores, questionnaire responses, and clinical flags. They offer limited capability to integrate multiple clinical indicators, such as symptom severity scores, questionnaire responses, and clinical flags, into systematic patient stratification frameworks. As a result, current solutions generally lack mechanisms to deterministically assign exercise tiers, therapy profiles, or diagnosis mappings to guide individualized treatment plans. Furthermore, few platforms incorporate automated identification of patients who may require modified therapy parameters or additional clinical oversight.

Another limitation is the absence of integrated safety and governance mechanisms in digital therapy systems. Many existing platforms lack structured guardrail checks, red-flag screening procedures, or automated review triggers identifying adverse symptoms, elevated risk indicators, or therapy regression during program execution. Similarly, mechanisms for monitoring patient adherence, capturing adverse events, and performing structured mid-program reassessments remain limited.

Existing systems further lack robust frameworks for generating structured longitudinal datasets that link patient inputs, therapy prescriptions, adherence patterns, and treatment outcomes. Without standardized data pipelines and auditable rule execution, it becomes difficult to evaluate treatment effectiveness across diverse patient populations or identify optimal therapy protocols.

Recent developments in artificial intelligence have introduced new approaches to digital health decision support; however, many AI-based systems rely on probabilistic machine learning or large language models that produce non-deterministic outputs. Such systems may lack transparency, reproducibility, and traceability, which are critical requirements for clinical decision-making, regulatory compliance, and safety auditing.

Accordingly, there remains a need for improved systems and methods for delivering pelvic floor physical therapy that expand access to treatment, systematically translate patient-specific clinical data into individualized therapy plans, enforce clinical governance and safety mechanisms, and generate structured outcome datasets suitable for longitudinal analysis. A system implementing deterministic clinical logic, auditable rule execution, and structured data collection would enable scalable, reproducible, and explainable personalization of PFPT interventions while improving patient engagement and clinical outcomes.

### SUMMARY

An objective of the present invention is to provide a system and method that enables systematic and consistent generation of personalized pelvic floor physical therapy care plans based on structured clinical inputs, thereby reducing variability associated with subjective clinical interpretation.

Another objective of the present invention is to provide a system and method that establishes a deterministic and transparent framework for clinical scoring and classification, ensuring reproducibility, auditability, and reliability in the assessment of patient condition and therapy assignment.

Another objective of the present invention is to provide a system and method that enables the integration of multiple validated clinical assessment instruments into a unified evaluation model, thereby facilitating comprehensive and multidimensional characterization of pelvic floor dysfunction.

Another objective of the present invention is to provide a system and method that includes a mechanism for mapping patient-specific clinical data to structured therapy tiers, exercise profiles, and diagnostic classifications, enabling standardized yet individualized treatment pathways.

Another objective of the present invention is to provide a system and method that incorporates risk-based patient stratification, thereby enabling differentiation of care intensity and facilitating early identification of patients requiring heightened clinical attention.

Another objective of the present invention is to provide a system and method that generates parameterized and adaptable therapeutic exercise programs, including progression criteria and adjunct recommendations, thereby improving treatment precision and patient adherence.

Another objective of the present invention is to provide a system and method that supports the personalization of therapeutic instruction delivery, including variations in cueing styles, thereby enhancing patient engagement and the effectiveness of therapy execution.

Another objective of the present invention is to provide a system and method that enable continuous, feedback-driven refinement of therapy plans by incorporating longitudinal outcome data, thereby supporting adaptive, evolving treatment strategies over time.

Another objective of the present invention is to provide a system and method that facilitates the structured collection and analysis of treatment outcome data, enabling the generation of clinically relevant insights and identification of patterns associated with therapeutic response.

Another objective of the present invention is to provide a system and method that incorporates robust clinical safety mechanisms, including guardrails, review checkpoints, and adverse event monitoring, thereby ensuring safe deployment of personalized therapy plans.

Yet another objective of the present invention is to provide a system and method that maintains end-to-end traceability and auditability between patient inputs, computed scores, applied rules, and generated outputs, thereby supporting clinical validation and regulatory compliance.

Yet another objective of the present invention is to provide a system and method that enables dynamic protocol modification and conditional adaptation of therapy plans, ensuring responsiveness to changing patient conditions without compromising predefined clinical constraints.

Yet another objective of the present invention is to provide a system and method that includes mechanisms for monitoring adherence and detecting regression across multiple clinical domains, thereby improving early intervention and long-term treatment outcomes.

Yet another objective of the present invention is to provide a system and method that supports the generation of structured clinical documentation, thereby reducing administrative burden and improving consistency in clinical reporting.

Yet another objective of the present invention is to provide a system and method that enable data-driven discovery of digital biomarkers and clinically significant patterns from aggregated outcome data, thereby advancing evidence-based pelvic floor therapy.

Yet another objective of the present invention is to provide a system and method capable of maintaining version-controlled clinical logic and decision frameworks, thereby ensuring consistency and traceability across evolving therapeutic protocols.

Yet another objective of the present invention is to provide a system and method that supports measurement of agreement between automated system-generated plans and clinician-reviewed plans, thereby enabling validation of system performance and supporting regulatory acceptance.

Yet another objective of the present invention is to provide a system and method that enables the generation of digital biomarkers from aggregated clinical, therapy, and outcome data, thereby facilitating the identification of statistically significant patterns associated with pelvic floor dysfunction and treatment response.

Yet another objective of the present invention is to provide a system and method that systematically transforms longitudinal clinical data into evidence-based insights, thereby supporting data-driven refinement of therapeutic strategies.

Yet another objective of the present invention is to provide a system and method that continuously establishes a closed-loop learning framework to improve future care plan generation, wherein insights derived from aggregated patient data are utilized to improve future care plan generation.

Yet another objective of the present invention is to provide a system and method that enables integration of population-level analysis with individual patient care, thereby bridging the gap between large-scale clinical evidence and personalized therapy delivery.

Yet another objective of the present invention is to provide a system and method that incorporates a mechanism for identifying predictive indicators of therapeutic outcomes, thereby enabling early intervention and improved clinical decision-making.

Yet another objective of the present invention is to provide a system and method that supports statistically grounded validation of treatment effectiveness, thereby enhancing clinical credibility and supporting evidence-based practice.

Yet another objective of the present invention is to provide a system and method that identifies emerging data patterns, thereby improving long-term system performance.

Yet another objective of the present invention is to provide a system and method that facilitates scalable learning across a population of patients, enabling the system to improve accuracy and effectiveness as more outcome data becomes available.

This and other objectives are achieved by providing a system and method for deterministic generation and adaptive management of a personalized pelvic floor physical therapy care plan as defined in the features of the independent claims. Additional advantageous embodiments and improvements of the invention are listed in the dependent claims. The use of expressions like “...aspect according to the invention” or “in one embodiment” or similar terminology is intended to refer to examples or embodiments consistent with the broadest scope of the invention as defined by the independent claims.

According to a first aspect, the present invention discloses a system for deterministic generation and adaptive management of a personalized pelvic floor physical therapy care plan. The system comprises an input module, a deterministic scoring module, an assignment module, a risk determination module, a care plan generation module, an outcome collection module, and a feedback module. The input module includes receiving patient-specific clinical data, including responses to a plurality of validated clinical assessment instruments. The deterministic scoring module computes one or more baseline clinical scores and one or more clinical flags from patient-specific clinical data using deterministic scoring functions. The assignment module assigns the patient to at least one exercise tier, at least one therapy profile, and one or more diagnosis codes based on baseline clinical scores and clinical flags. The risk determination module includes determining a patient's risk classification based on the exercise tier, therapy profile, diagnosis codes, baseline clinical scores, and clinical flags. The care plan generation module includes generating an individualized pelvic floor physical therapy care plan based on the patient's risk classification. The outcome collection module collects longitudinal treatment outcome data generated by the execution of the individualized care plan. The feedback module updates the baseline clinical scores, clinical flags, exercise tier, therapy profile, diagnosis codes, and patient risk classification based on longitudinal treatment outcome data for subsequent care plan generation. Therefore, the system enables systematic generation of individualized personalized pelvic floor therapy plans and facilitates iterative refinement of care planning based on collected treatment outcome data.

In an embodiment of the present invention, the plurality of validated clinical assessment instruments includes at least an incontinence instrument, a pain instrument, a prolapse instrument, a bowel function instrument, or a mental health screening instrument. This enables assessment of multiple functional domains relevant to pelvic floor dysfunction.

In another embodiment of the present invention, the deterministic scoring module computes severity classifications by mapping baseline clinical scores to predefined severity ranges and generating composite scores derived from multiple instrument values. This enables consistent classification of symptom severity based on predefined score ranges and aggregated assessment results.

In another embodiment of the present invention, the clinical flags include at least one of constipation indicators, depression indicators, prolapse indicators, and pain severity indicators. This enables identification of clinically relevant condition indicators for consideration during therapy planning.

In another embodiment of the present invention, the deterministic scoring functions include a composite pain score derived from reusing a validated instrument pain item as both a component of the validated instrument score and a component of a custom composite score, while preserving the original instrument structure. This enables the incorporation of instrument-driven pain information into additional scoring constructs while maintaining compatibility with the original validated instrument structure.

In another embodiment of the present invention, the assignment module assigns the exercise tier based on predefined severity thresholds. This is enabled based on predefined severity thresholds.

In another embodiment of the present invention, the care plan generation module generates parameterized therapeutic exercises including repetition counts, hold durations, relaxation durations, session frequency, and progression criteria. This enables precise and reproducible prescription of therapeutic exercises tailored to individual patient needs.

In another embodiment of the present invention, the assignment module further assigns adjunct therapy recommendations based on the clinical flags and severity thresholds. This enables holistic treatment planning that goes beyond core exercise interventions and is based on detected clinical indicators.

In another embodiment of the present invention, the assignment module assigns diagnosis codes based on predefined mappings between clinical score ranges and diagnostic classifications. This enables standardized clinical classification and improved interoperability with healthcare systems.

In another embodiment of the present invention, the risk determination module classifies patients into at least standard risk and elevated risk categories based on combinations of pain severity, prolapse indicators, and mental health screening results. This enables categorization of patients into predefined risk levels based on multiple clinical indicators.

In another embodiment of the present invention, the care plan generation module personalizes exercise instructions based on a received patient cue preference. This enables improved patient engagement and adherence through individualized instruction.

In another embodiment of the present invention, patient cue preference includes at least biologic cueing, imaginative cueing, breath-based cueing, or contraction cueing. This enables the selection of appropriate instruction modalities to enhance patient comprehension and correct execution of therapeutic exercises.

In another embodiment of the present invention, the system further comprises a documentation module that generates structured SOAP clinical encounter documentation from the individualized care plan. This enables an efficient process with a reduced administrative burden.

In another embodiment of the present invention, the system further comprises a clinical review module to enforce clinical guardrail checks during plan modifications. This enables safe and compliant therapy delivery through enforced clinical validation checks.

In another embodiment of the present invention, the clinical guardrail checks include at least one of a minimum review duration verification, red flag screening verification, and clinical documentation requirements. This enables verification of critical clinical conditions and compliance requirements to therapy execution.

In another embodiment of the present invention, the longitudinal outcome data includes at least one symptom progression, treatment adherence data, functional outcome measures, or repeated clinical assessment results. This enables longitudinal tracking of patient progress and treatment effectiveness.

In another embodiment of the present invention, the system further comprises embedding a clinical logic version identifier within generated care plans and outcome records, and updating the version identifier when scoring rules, assignment thresholds, diagnosis mappings, or therapy parameters are modified. This enables traceability and consistency across evolving clinical decision frameworks.

In another embodiment of the present invention, the input module further enforces field-level validation with conditional instrument completion requirements to prevent incomplete instrument scoring from influencing deterministic scoring outputs. This enables data integrity by preventing incomplete or invalid inputs from influencing clinical decisions.

In another embodiment of the present invention, the assignment module executes conditional logic branches that modify the individualized care plan based on threshold values of the baseline clinical scores or clinical flags. This enables modification of therapy plan parameters based on predefined threshold conditions.

In another embodiment of the present invention, the system further comprises a substitute for one or more therapeutic actions with alternative actions while preserving associated parameter values upon detection of a predefined condition. This enables flexible modification of therapy actions while preserving treatment structure and parameters.

In another embodiment of the present invention, the feedback module performs statistical analysis comprising descriptive statistics, effect sizes using Cohen's d, and statistical significance using Welch's t-test. This enables evidence-based identification of clinically meaningful patterns using statistically validated methods.

In another embodiment of the present invention, a biomarker generation module partitions patients into subgroups based on combinations of baseline clinical scores, behavioral attributes, and therapy parameters for statistical analysis for biomarker generation. This enables granular analysis of treatment response across distinct patient populations.

In yet another embodiment of the present invention, the system maintains an auditable linkage between patient-specific input data, computed clinical scores, applied rules, generated care plan parameters, collected outcome data, and updated decision parameters. This enables end-to-end traceability of clinical decisions for validation and regulatory compliance.

In yet another embodiment of the present invention, the system further comprises a mid-program clinical checkpoint at a predetermined interval that re-administers a subset of baseline clinical instruments, computes deltas relative to baseline, classifies a concern level using deterministic thresholds, and generates triggered review flags for a supervising clinician. This enables the identification of predefined concern conditions during mid-program reassessment.

In yet another embodiment of the present invention, the clinical review module enforces two categories of review flags: a) always-review flags deterministically generated at care plan creation based on baseline instrument scores and risk factors, and b) triggered flags dynamically generated during post-treatment monitoring based on longitudinal score changes, adherence patterns, or adverse event reports. This enables structured clinician oversight by systematically identifying cases requiring clinical attention at different stages of therapy.

In yet another embodiment of the present invention, the system further comprises a daily adherence logging module window analysis. This enables continuous evaluation of patient adherence behavior across defined time intervals, thereby supporting detection of adherence patterns during therapy execution.

In yet another embodiment of the present invention, the patient entries include completion status and date, retroactive entries are permitted with a backfill marker, rolling adherence rates are computed over a defined window, adherence concern flags are generated when thresholds are not met, and month-level aggregation is supported. This enables quantitative evaluation of adherence patterns and early detection of non-compliance trends.

In yet another embodiment of the present invention, the system comprises a structured adverse event capture module with severity-gated escalation. This enables structured capture and escalation of reported adverse events.

In yet another embodiment of the present invention, the structured adverse event capture module collects new-symptom indicators, free-text descriptions, and numeric severity scales, elevates concern levels when severity exceeds a predefined threshold, generates adverse event review flags, and incorporates the adverse event data into outcome records for biomarker analysis. This enables prioritization and escalation of clinically significant adverse events based on severity thresholds.

In still another embodiment of the present invention, the system further comprises a multi-domain regression detection module for a post-treatment checkpoint. This enables identification of deterioration across multiple clinical domains for proactive intervention.

In still another embodiment of the present invention, the multi-domain regression detection module computes domain-specific deltas independently for a plurality of clinical instruments, applies worsening thresholds for each domain, and generates domain-specific review flags when regression exceeds the respective thresholds. This enables granular detection of regression across multiple clinical domains for targeted intervention.

In still another embodiment of the present invention, an agreement analysis module computes an AI-clinician agreement rate from aggregated outcome records by comparing the number of care plans approved without clinician modifications to the total number of completed care plans. It presents the agreement rate as a regulatory compliance metric. This enables quantitative validation of system-generated care plans against clinician decisions.

According to a second aspect, the present invention discloses a method for deterministic generation and adaptive management of a personalized pelvic floor physical therapy care plan. The method comprises the following steps: (a) receiving patient-specific clinical data, including responses to a plurality of validated clinical assessment instruments; (b) computing one or more baseline clinical scores and one or more clinical flags using deterministic scoring functions; (c) assigning a patient to at least one exercise tier, at least one therapy profile, and one or more diagnosis codes based on the baseline clinical scores and clinical flags; (d) determining a patient risk classification based on the exercise tier, therapy profile, diagnosis codes, baseline clinical scores, and clinical flags; (e) generating an individualized pelvic floor physical therapy care plan based on the patient risk classification; (f) collecting longitudinal treatment outcome data generated from execution of the individualized care plan; and (g) updating the baseline clinical scores, clinical flags, exercise tier, therapy profile, diagnosis codes, and patient risk classification based on the longitudinal treatment outcome data for subsequent care plan generation. This method enforces a structured, repeatable process for delivering personalized, adaptive pelvic floor therapy through deterministic clinical workflows.

According to the third aspect, the present invention discloses a system for deterministic generation and adaptive management of a personalized pelvic floor physical therapy care plan. The system comprises an input module, a deterministic scoring module, an assignment module, a risk determination module, a care plan generation module, an outcome collection module, a biomarker generation module, and a feedback module. The input module includes receiving patient-specific clinical data, including responses to a plurality of validated clinical assessment instruments. The deterministic scoring module computes one or more baseline clinical scores and one or more clinical flags from patient-specific clinical data using deterministic scoring functions. The assignment module assigns the patient to at least one exercise tier, at least one therapy profile, and one or more diagnosis codes based on baseline clinical scores and clinical flags. The risk determination module includes determining a patient's risk classification based on the exercise tier, therapy profile, diagnosis codes, baseline clinical scores, and clinical flags. The care plan generation module includes generating an individualized pelvic floor physical therapy care plan based on the patient's risk classification. The outcome collection module collects longitudinal treatment outcome data generated by the execution of the individualized care plan. The biomarker generation module includes receiving aggregated clinical, therapy, and outcome data and generating digital biomarkers by applying statistical analysis to detect statistically significant clinical patterns. The feedback module updates the baseline clinical scores, clinical flags, exercise tier, therapy profile, diagnosis codes, and patient risk classification, based on generated digital biomarkers and longitudinal treatment outcome data, for subsequent care plan generation. Therefore, the system enables refinement of personalized pelvic floor therapy plans using deterministic clinical logic while continuously improving outcomes by integrating outcome-derived statistical biomarkers into the care-planning process.

### BRIEF DESCRIPTION OF DRAWINGS

The drawings illustrate the design and utility of embodiments of the present invention, in which common reference numerals refer to similar elements. To better appreciate the advantages and objects of the embodiments of the present invention, reference should be made to the accompanying drawings that illustrate these embodiments. However, the drawings depict only some embodiments of the invention and should not be taken as limiting its scope.

Various aspects, as well as embodiments of the present invention, are better understood by referring to the following detailed description. To better understand the invention, the detailed description should be read in conjunction with the drawings. With this caveat, embodiments of the invention will be described and explained with additional specificity and detail through the use of the accompanying drawings in which:

FIG. 1 illustrates a block diagram of a system for deterministic generation and adaptive management of a personalized pelvic floor physical therapy care plan in accordance with an exemplary embodiment of the present invention;

FIG. 2 illustrates a block diagram of an input and validation flow of the system 100 in accordance with an exemplary embodiment of the invention;

FIG. 3 illustrates a block diagram of a deterministic scoring module associated with the system in accordance with an exemplary embodiment of the present invention;

FIG. 4 illustrates a block diagram of an assignment module associated with the system in accordance with an exemplary embodiment of the invention;

FIG. 5 illustrates a block diagram of a risk determination module associated with the system in accordance with an embodiment of the present invention;

FIG. 6 illustrates a block diagram of a care plan generation module associated with the system in accordance with an exemplary embodiment of the present invention;

FIG. 7 illustrates a block diagram of an outcome collection module associated with the system in accordance with an exemplary embodiment of the present invention;

FIG. 8 illustrates a block diagram of a feedback module associated with the system in accordance with an exemplary embodiment of the present invention;

FIG. 9 illustrates a block diagram of a biomarker generation module associated with the system in accordance with an exemplary embodiment of the present invention;

FIG. 10 illustrates a block diagram of a clinical review module associated with the system in accordance with an exemplary embodiment of the present invention;

FIG. 11 illustrates a block diagram of a documentation module associated with the system in accordance with an exemplary embodiment of the present invention;

FIG. 12 illustrates a block diagram of a protocol modification module associated with the system in accordance with an exemplary embodiment of the present invention;

FIG. 13 illustrates a block diagram of a regression detection module associated with the system in accordance with an exemplary embodiment of the present invention;

FIG. 14 illustrates a block diagram of an agreement analysis module associated with the system in accordance with an exemplary embodiment of the present invention;

FIG. 15 illustrates a block diagram depicting a mid-program clinical checkpoint in accordance with an exemplary embodiment of the present invention;

FIG. 16 illustrates a flowchart of a method for generating a personalized pelvic floor physical therapy care plan in accordance with an exemplary embodiment of the present invention;

FIG. 17 illustrates a block diagram of an end-to-end operational flow for generating and refining a personalized pelvic floor physical therapy care plan in accordance with an exemplary embodiment of the present invention;

FIG. 18 illustrates a block diagram of a complete care plan generation through deterministic scoring and assignment processes in accordance with an exemplary embodiment of the present invention;

FIG. 19 illustrates a decision flow for an exercise tier assignment in accordance with an exemplary embodiment of the present invention;

FIG. 20 illustrates a block diagram of a rule-based mapping between trigger conditions and adjunct therapy recommendations in accordance with an exemplary embodiment of the present invention;

FIG. 21 illustrates a block diagram of a prenatal protocol modification workflow in accordance with an exemplary embodiment of the present invention;

FIG. 22 illustrates a block diagram of a risk evaluation workflow in accordance with an exemplary embodiment of the present invention;

FIG. 23 illustrates a block diagram of a longitudinal data lifecycle workflow in accordance with an exemplary embodiment of the present invention;

FIG. 24 illustrates a block diagram of an analytical workflow for generating digital biomarkers in accordance with an exemplary embodiment of the present invention;

FIG. 25 illustrates a block diagram of a biomarker candidate architecture in accordance with an exemplary embodiment of the present invention;

FIG. 26 illustrates a block diagram of a closed-loop evidence generation architecture in accordance with an exemplary embodiment of the present invention;

FIG. 27 illustrates a block diagram of a clinical review flag architecture in accordance with an exemplary embodiment of the present invention;

FIG. 28 illustrates a longitudinal reassessment workflow 2800 in accordance with the present invention;

FIG. 29 illustrates a multidomain concern aggregation workflow 2900 in accordance with the present invention; and

FIG. 30 illustrates a domain-agnostic core pipeline 3000 in accordance with the present invention.

### DETAILED DESCRIPTION

### Definitions

As used herein, “input data” refers to any structured or unstructured data received by the system, including, but not limited to, questionnaire responses, sensor data, clinical observations, user-reported outcomes, and externally provided datasets. “Structured data” refers to data organized according to a predefined schema, format, or model, while “unstructured data” refers to data that does not conform to a predefined structure.

As used herein, “scoring function” refers to any algorithm, rule set, or computational process that evaluates one or more inputs to generate a score, classification, or derived value. A “deterministic scoring function” refers to a scoring function that produces consistent outputs for identical inputs under defined conditions. A “rule” refers to a defined condition-action relationship used to evaluate data or determine system outputs.

As used herein, “care plan object,” “output object,” or “structured output” refers to a machine-readable data structure that includes one or more recommended actions, classifications, or parameters generated by the system. Such structures may include associated metadata, identifiers, and execution parameters and may be stored, transmitted, or processed by other computing systems.

As used herein, “traceability” or “auditability” refers to the ability to identify, reconstruct, and analyze the sequence of inputs, rules, and intermediate computations that resulted in a given system output. This may include maintaining logs, rule execution traces, or version-controlled rule sets.

As used herein, “domain” refers to any field or application area in which structured input data, scoring functions, and rule-based outputs can be defined. The system is not limited to any specific domain, and references to particular domains are illustrative rather than limiting.

As used herein, “system” may refer to a single computing device, a collection of computing devices, or a distributed architecture comprising multiple interconnected components. The term encompasses both centralized and decentralized implementations.

The foregoing description of the embodiments of the present invention has been provided for purposes of illustration and description. It is not intended to be exhaustive or to limit the invention to the precise forms disclosed. Many modifications and variations will be apparent to practitioners skilled in the art in light of the above disclosure. It is intended that the scope of the invention be defined by the claims appended hereto and their equivalents.

In the foregoing specification, specific embodiments have been described. However, it will be appreciated that various modifications and changes may be made without departing from the broader scope of the invention as outlined in the claims. Accordingly, the specification and drawings are to be regarded in an illustrative rather than restrictive sense.

The figures illustrate the architecture, functionality, and operation of possible implementations of systems, methods, and computer program products in accordance with various embodiments of the present invention. In this regard, each block in the figures may represent a module, segment, or portion of instructions that comprises one or more executable instructions for implementing specified logical functions. It should also be noted that, in some alternative implementations, the functions noted in the blocks may occur out of the order illustrated in the figures. For example, two blocks shown in succession may, in fact, be executed substantially concurrently, or the blocks may sometimes be executed in reverse order, depending upon the functionality involved.

The terms comprising, including, containing, or any variation thereof, are intended to cover a non-exclusive inclusion such that a process, method, article, or apparatus that comprises a list of elements does not include only those elements but may include other elements not expressly listed or inherent to such process, method, article, or apparatus.

Furthermore, the terms “a” or “an”, as used herein, are defined as one or more unless explicitly stated otherwise. The term plurality, as used herein, is defined as two or more unless explicitly stated otherwise.

The term another, as used herein, is defined as at least a second or more. The terms having, including, and comprising are defined as open-ended terms (i.e., meaning “including, but not limited to”) unless otherwise noted.

The term coupled, as used herein, is defined as connected, although not necessarily directly and not necessarily mechanically. The term configured to describes hardware and/or software that is adapted, arranged, capable, or programmed to perform a function.

The terms “substantially”, “approximately”, or similar terms may be used to describe values or characteristics and are intended to account for acceptable tolerances or variations as understood by those skilled in the art.

It will be further understood that when an element is referred to as being connected, coupled, or responsive to another element, it may be directly connected or coupled to the other element, or intervening elements may be present.

Reference throughout this specification to one embodiment, an embodiment, or similar language means that a particular feature, structure, or characteristic described may be included in at least one embodiment of the present invention. Thus, appearances of such phrases throughout this specification do not necessarily refer to the same embodiment.

It should also be understood that the described features, structures, or characteristics of the invention may be combined in any suitable manner in one or more embodiments. In the following description, numerous specific details may be provided to give a thorough understanding of embodiments of the invention. One skilled in the relevant art will recognize, however, that the invention may be practiced without one or more of the specific details or with other methods, components, materials, or structures. In some instances, well-known structures, materials, or operations may not be shown or described in detail to avoid obscuring aspects of the invention.

The Abstract of the Disclosure is provided to allow the reader to ascertain the nature of the technical disclosure quickly. It is submitted with the understanding that it will not be used to interpret or limit the scope or meaning of the claims.

In addition, in the foregoing Detailed Description, various features may be grouped in embodiments to streamline the disclosure. This method of disclosure should not be interpreted as reflecting an intention that claimed embodiments require more features than are expressly recited in each claim. Rather, inventive subject matter may lie in less than all features of a single disclosed embodiment.

Thus, the following claims are hereby incorporated into the Detailed Description, with each claim standing on its own as a separately claimed subject matter.

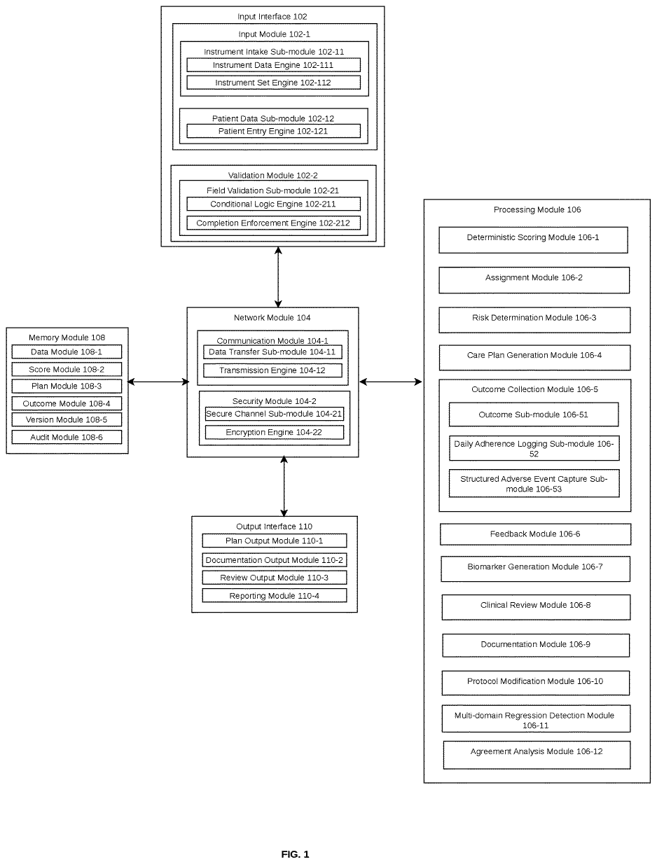

FIG. 1 illustrates a block diagram of a system 100 for deterministic generation and adaptive management of a personalized pelvic floor physical therapy care plan in accordance with an embodiment of the present invention. The system 100 comprises an input interface 102, a network module 104, a processing module 106, a memory module 108, and an output interface 110, each operatively coupled to enable data exchange and coordinated execution of clinical decision logic in accordance with the claimed system.

In some cases, the system 100 is implemented using one or more computing devices comprising the processing module 106, the memory module 108, and the network module 104. The processors execute instructions stored in memory to perform the operations described herein. The system's 100 components may be implemented using software, hardware, firmware, or any combination thereof. In certain implementations, the system 100 operates in a networked environment, including cloud-based or distributed computing environments, where one or more modules are executed across multiple physical or virtual machines.

As used herein, the terms “module,” “sub-module,” “engine,” and “sub-engine” refer to logical, functional, or structural components of the system 100 that may be implemented in hardware, software, firmware, or any combination thereof. A module may comprise one or more sub-modules or engines, and an engine may include one or more sub-engines. These elements may be distributed across multiple computing devices or consolidated within a single system 100. The delineation of such components is provided for clarity of description and does not limit the implementation to any specific architecture or configuration.

The input interface 102 includes an input module 102-1 and a validation module 102-2. The input module 102-1 is configured to receive patient-specific clinical data, including responses to a plurality of validated clinical assessment instruments. The validated clinical assessment instruments include an incontinence instrument, a pain instrument, a prolapse instrument, a bowel function instrument, and a mental health screening instrument. The incontinence instrument is configured to capture the frequency, severity, and impact of urinary leakage on daily activities. The pain instrument is configured to quantify pain intensity, distribution, and associated functional limitations. The prolapse instrument is configured to assess pelvic organ prolapse symptoms and related distress. The bowel function instrument is configured to evaluate stool consistency, frequency, and associated gastrointestinal symptoms, and the mental health screening instrument is configured to assess psychological indicators such as depression or anxiety. Each instrument in the validated clinical assessment is structured with predefined question sets and scoring rules used for downstream processing.

In some cases, the system 100 includes validation and error-handling mechanisms to address incomplete, inconsistent, or invalid input data. Such mechanisms may include input validation rules, default value assignment, exclusion of invalid data points, or generation of alerts requesting additional input. These processes ensure that downstream deterministic scoring and rule execution operate on reliable and standardized data.

The input module 102-1 further comprises an instrument intake sub-module 102-11 and a patient data sub-module 102-12. The instrument intake sub-module 102-11 further comprises an instrument data engine 102-111 and an instrument set engine 102-112. The instrument intake sub-module 102-11 is configured to perform the functions of its constituent sub-components, including the instrument data engine 102-111, which captures structured instrument responses. These instruments are organized into clinically relevant categories to ensure comprehensive coverage of pelvic floor conditions and associated comorbidities. Within the pelvic floor female category, the instrument intake sub-module 102-11 receives inputs corresponding to instruments including ICIQ, FLUTS, GUPI, POPDI-6, PHQ-2, and Bristol stool scale, each instrument capturing specific symptom domains such as urinary incontinence severity, lower urinary tract symptoms, pelvic pain, prolapse distress, mental health screening, and bowel function characteristics.

Within the pelvic floor male category, the instrument intake sub-module 102-11 receives inputs from ICIQ-MLUTS, IPSS, NIH-CPSI, and PHQ-2, thereby capturing male-specific lower urinary tract symptoms, prostate-related symptom severity, chronic pelvic pain indices, and mental health indicators. For musculoskeletal and low back pain conditions, the instrument intake sub-module 102-11 receives inputs from OSPRO-YF, FABQ, PSEQ, ODI, and NPRS, enabling evaluation of pain intensity, fear-avoidant behaviors, functional disability, and self-efficacy. For vestibular rehabilitation, the instrument intake sub-module 102-11 receives inputs from DHI and BPPV screening instruments to assess dizziness-related handicap and positional vertigo indicators.

Although specific embodiments are described with respect to pelvic floor, musculoskeletal, vestibular, and oncology rehabilitation domains, the disclosed system architecture is not limited to these applications. The system may be applied to any domain in which structured input data, deterministic scoring functions, and rule-based intervention assignment can be defined. For each such domain, implementation requires substitution of domain-specific instruments, scoring functions, and intervention libraries within the deterministic framework described herein. Accordingly, the described embodiments are illustrative and not limiting, and the system is adaptable to a wide range of clinical and non-clinical decision-making environments.

The instrument set engine 102-112 is configured to manage instrument groupings, enforce completeness across instrument sets, and organize incoming responses into structured formats suitable for downstream deterministic scoring. For example, a patient completing a pelvic floor intake questionnaire may provide responses to a validated incontinence instrument and a mental health screening instrument, which are captured and structured by the instrument data engine 102-111 and grouped via the instrument set engine 102-112.

The patient data sub-module 102-12 further comprises a patient entry engine 102-121. The patient entry engine 102-121 is configured to receive demographic data, symptom descriptions, behavioral attributes, and patient cue preferences, including biologic cueing, imaginative cueing, breath-based cueing, or contraction cueing. For instance, a patient may indicate a preference for breath-based cueing during exercise instruction, which is then stored and later used to personalize the care plan. The input interface 102 further includes the validation module 102-2 configured to enforce field-level validation with conditional instrument completion requirements. The validation module 102-2 comprises a field validation sub-module 102-21, which includes a conditional logic engine 102-211 and a completion enforcement engine 102-212.

The conditional logic engine 102-211 is configured to enforce completion dependencies between instrument items, and the completion enforcement sub-engine 102-212 is configured to prevent incomplete or inconsistent data from influencing deterministic scoring outputs. For example, if a required pain severity item is missing, the completion enforcement sub-engine 102-212 may block progression until the required input is provided, thereby ensuring scoring integrity. The system 100 further includes a network module 104 configured to facilitate communication between components of system 100 and one or more external devices, including patient-operated devices, clinician interfaces, and remote computing devices. The network module 104 functions as a communication layer that enables bidirectional transmission of data required for operation of the system 100, including patient-specific clinical data, generated care plans, longitudinal outcome data, and system-generated outputs. The network module 104 is operatively coupled with the input interface 102, the processing module 106, the memory module 108, and the output interface 110, thereby enabling coordinated data exchange across the system 100.

The network module 104 comprises a communication Module 104-1 and a security module 104-2. The communication module 104-1 is configured to manage data transmission between client devices and backend components. The communication module 104-1 includes a data transfer sub-module 104-11 configured to structure, package, and route data for transmission across one or more communication networks. The data transfer sub-module 104-11 supports transmission of various data types. The communication module 104-1 further includes a transmission engine 104-12 configured to execute the actual transmission of data packets over wired or wireless communication channels, including internet-based networks, local area networks, or mobile communication networks. The transmission engine 104-12 ensures reliable data delivery between components of the system 100, including synchronization of data between client devices and the memory module 108.

The network module 104 further includes the security module 104-2 configured to ensure secure transmission of sensitive clinical and patient-specific data. The security module 104-2 includes a secure channel sub-module 104-21 configured to establish secure communication channels between components of system 100 and external devices. These secure channels may be implemented using standardized communication protocols that support the confidentiality and integrity of transmitted data. The security module 104-2 further includes an encryption engine 104-22 configured to encrypt data before transmission and to decrypt data upon receipt, thereby protecting patient data from unauthorized access during transmission. The encryption engine 104-22 may apply encryption algorithms to ensure that data such as clinical assessment responses, care plan parameters, adherence logs, and adverse event reports are transmitted securely across networks.

The network module 104 is further configured to support real-time and asynchronous communication, enabling continuous data exchange between patients and the system 100. For example, patient adherence entries captured through a mobile application may be transmitted via the communication module 104-1 using the transmission engine 104-12, while the security module 104-2 ensures that the data is encrypted and transmitted through a secure channel established by the secure channel sub-module 104-21. Upon receipt, the data is routed to the processing module 106, where it is processed by the outcome collection module 106-5 and stored in the memory module 108.

In another illustrative scenario, an individualized pelvic floor physical therapy care plan generated by the care plan generation module 106-4 may be transmitted from the processing module 106 to a patient device through the network module 104. The data transfer sub-module 104-11 structures the care plan data, the transmission engine 104-12 transmits the data over a network, and the security module 104-2 ensures that the transmission is encrypted and secure. Similarly, review flags generated by the clinical review module 106-8 or updated risk classifications generated by the feedback module 106-6 may be transmitted to clinician interfaces for review and intervention.

Through these coordinated functions, the network module 104 enables seamless, secure, and reliable communication across the system 100, ensuring that all data required for patient assessment, care plan execution, outcome monitoring, and feedback-driven updates are transmitted efficiently while maintaining the confidentiality and integrity of sensitive clinical information. The processing module 106 comprises a plurality of coordinated submodules, including a deterministic scoring module 106-1, an assignment module 106-2, a risk determination module 106-3, a care plan generation module 106-4, an outcome collection module 106-5, a feedback module 106-6, a biomarker generation module 106-7, a clinical review module 106-8, a documentation module 106-9, a protocol modification module 106-10, a regression detection module 106-11, and an agreement analysis module 106-12.

The processing module 106 forms the core computational layer of the system 100 and includes multiple coordinated modules implementing the claimed functionality. Among these, the deterministic scoring module 106-1 is configured to compute one or more baseline clinical scores and one or more clinical flags from the patient-specific clinical data using deterministic scoring functions, as recited in the claims. The deterministic scoring module 106-1 operates on structured responses received from the input interface 102, including responses to a plurality of validated clinical assessment instruments such as an incontinence instrument, a pain instrument, a prolapse instrument, a bowel function instrument, and a mental health screening instrument. The deterministic nature of the scoring ensures that identical input data consistently produce identical outputs, thereby supporting reproducibility, auditability, and clinical transparency.

The system 100 transforms structured and unstructured input data, including questionnaire responses, clinical observations, and user-reported outcomes, into standardized intermediate representations and subsequently into structured care plan objects and outcome records. This transformation constitutes a technical data-processing operation in which input data is normalized, evaluated according to deterministic rules, and converted into machine-readable outputs that can be stored, transmitted, and further processed by computing systems.

The system 100 provides a technical improvement to computer-implemented clinical decision systems by enforcing deterministic, rule-bound execution across structured clinical data inputs. Unlike non-deterministic machine learning systems, the disclosed architecture ensures reproducible outputs for identical inputs and enables full traceability of decision pathways. This deterministic processing reduces computational ambiguity, enables version-controlled execution of clinical logic, and improves system auditability, thereby enhancing the functioning of computer-based decision systems rather than merely automating an abstract clinical process.

The deterministic scoring module 106-1 includes a score computation sub-module configured to compute baseline clinical scores using predefined scoring functions associated with each validated clinical assessment instrument. These scoring functions preserve the original structure of the validated instruments while enabling reuse of specific instrument items for additional composite scoring. In particular, the score computation sub-module is configured to compute composite scores derived from multiple instrument values, including a composite pain score derived from reuse of a validated instrument pain item as both a component of the validated instrument score and a component of a custom composite score, while preserving the integrity of the original instrument structure. For example, a numerical pain rating provided within a validated pain instrument may contribute to both the overall instrument score and a cross-domain composite score that integrates pain with functional impairment indicators.

The deterministic scoring module 106-1 further includes a classification sub-module configured to compute severity classifications by mapping baseline clinical scores to predefined severity ranges. These severity ranges are defined according to clinically established thresholds and may include categories such as mild, moderate, and severe. The classification sub-module applies deterministic mapping rules to ensure consistent categorization of patient condition severity across different instruments and composite scores. For instance, a score from a prolapse instrument may be mapped to a moderate-severity range based on predefined thresholds, which in turn influences downstream assignment and risk determination processes.

In addition, the deterministic scoring module 106-1 includes a flag sub-module configured to generate one or more clinical flags based on the computed baseline clinical scores and underlying instrument responses. The flag sub-module evaluates specific conditions and thresholds to identify clinically relevant indicators, including but not limited to constipation indicators derived from bowel function responses, depression indicators derived from mental health screening results, prolapse indicators derived from prolapse instrument scores, and pain severity indicators derived from pain instrument responses. These clinical flags are generated using deterministic logic to ensure that the presence or absence of each flag is directly attributable to specific input data and predefined criteria. For example, a patient reporting infrequent bowel movements and straining may trigger a constipation indicator flag. In contrast, elevated responses on a mental health screening instrument may trigger a depression indicator flag.

The outputs of the deterministic scoring module 106-1, including baseline clinical scores, severity classifications, composite scores, and clinical flags, are structured and stored for subsequent processing by downstream modules, such as the assignment module 106-2 and the risk determination module 106-3. These outputs form the foundational data layer upon which exercise tier assignment, therapy profile selection, diagnosis code mapping, and patient risk classification are based. By employing deterministic scoring functions, predefined severity mappings, and explicit flag-generation criteria, the deterministic scoring module 106-1 ensures consistency, traceability, and clinical interpretability across all stages of system 100.

The integration of deterministic scoring functions with cross-instrument composite scoring, domain-parameterized intervention assignment, and outcome-driven biomarker feedback is not a predictable combination of known elements. Conventional systems typically treat intake, scoring, intervention assignment, and outcome tracking as independent processes. In contrast, the present system enforces a unified execution pipeline in which each stage operates under deterministic constraints and directly informs subsequent stages, thereby producing synergistic improvements in reproducibility, auditability, and clinical standardization.

For example, in a representative implementation, a patient may complete a set of validated clinical assessment instruments, including a pain instrument and a bowel function instrument. The score computation sub-module calculates individual instrument scores and a composite pain score incorporating selected pain-related items. The classification sub-module maps these scores to severity categories, such as moderate pain severity and mild bowel dysfunction. Concurrently, the flag Sub-module generates a pain severity indicator and a constipation indicator based on predefined thresholds. These computed scores and flags are then provided to the assignment module 106-2 to determine an appropriate exercise tier and therapy profile, thereby enabling a data-driven and reproducible pathway for personalized care plan generation.

The processing module 106 further includes an assignment module 106-2 configured to assign the patient to at least one exercise tier, at least one therapy profile, and one or more diagnosis codes based on the baseline clinical scores and clinical flags generated by the deterministic scoring module 106-1. The assignment module 106-2 operates using predefined mappings, threshold-based rules, and deterministic logic structures to ensure consistent and reproducible assignment outcomes. The assignment module 106-2 serves as a translation layer that converts quantified clinical insights, including severity classifications and clinical flags, into structured therapeutic categorizations that directly inform care plan generation.

The assignment module 106-2 includes a tier sub-module configured to assign the patient to an exercise tier based on predefined severity thresholds derived from baseline clinical scores and associated severity classifications. The exercise tiers correspond to structured levels of therapeutic intensity, progression readiness, and functional capability, and may include categories such as low-intensity, moderate-intensity, and advanced-intensity tiers. The tier sub-module applies deterministic threshold logic to ensure that patients with higher severity classifications or elevated clinical risk indicators are assigned to lower-intensity tiers. In comparison, patients with lower severity classifications may be assigned to higher-intensity tiers. For example, a patient with a severe pain severity classification and multiple clinical flags may be assigned to a low-intensity exercise tier to reduce the risk of exacerbation during therapy.

The assignment module 106-2 further includes a profile sub-module configured to assign the patient to a therapy profile based on combinations of baseline clinical scores, clinical flags, and identified symptom domains. The therapy profiles are structured groupings of therapeutic approaches tailored to specific clinical presentations, such as pain-dominant, prolapse-focused, bowel dysfunction, or mixed-condition profiles. The profile sub-module uses deterministic mapping rules to associate specific combinations of scores and flags with predefined therapy profiles. For instance, the presence of elevated pain severity indicators combined with depression indicators may result in assignment to a therapy profile emphasizing pain modulation and behavioral support strategies.

The assignment module 106-2 also includes a diagnosis sub-module configured to assign one or more diagnosis codes based on predefined mappings between clinical score ranges, severity classifications, and diagnostic classifications. The diagnosis codes may correspond to standardized diagnostic coding regularities and are deterministically derived from clinical score thresholds and instrument-specific criteria. The diagnosis sub-module ensures that diagnosis assignments are consistent with the quantified clinical data and aligned with established diagnostic frameworks. For example, a prolapse instrument score exceeding a defined threshold may map to a corresponding prolapse diagnosis code. In contrast, a combination of bowel dysfunction indicators may map to a constipation-related diagnosis code.

In addition, the assignment module 106-2 includes an adjunct sub-module configured to assign adjunct therapy recommendations based on clinical flags, severity thresholds, and identified risk factors. The adjunct therapy recommendations may include supplemental therapeutic actions or supportive interventions that complement the primary exercise-based care plan, such as behavioral modifications, relaxation techniques, or additional clinical follow-up recommendations. The adjunct sub-module evaluates the presence of specific clinical flags and applies deterministic rules to determine whether adjunct therapies should be incorporated. For example, the presence of a depression indicator may trigger an adjunct recommendation for mental health support strategies, while a constipation indicator may trigger dietary or bowel management recommendations.

The assignment module 106-2 further includes a conditional logic sub-module configured to execute conditional logic branches that modify assignments based on threshold values of baseline clinical scores or the presence of specific clinical flags. The conditional logic sub-module enables dynamic adjustment of assignment outcomes by applying rule-based branching logic that refines or overrides initial assignments when predefined conditions are met. This includes modifying exercise-tier assignments, adjusting therapy-profile selection, altering diagnosis-code prioritization, or adding adjunct-therapy recommendations. The conditional logic sub-module ensures that assignment outputs are responsive to clinically significant combinations of factors rather than relying solely on independent score thresholds. For example, a patient initially assigned to a moderate-intensity exercise tier based on overall severity may be reassigned to a lower-intensity tier if both a high pain severity indicator and a prolapse indicator are present, thereby reducing the risk of adverse outcomes.

The outputs generated by assignment module 106-2, including the assigned exercise tier, therapy profile, diagnosis codes, and adjunct therapy recommendations, are structured and transmitted to downstream modules, such as the risk determination module 106-3 and the care plan generation module 106-4. These outputs serve as critical inputs for determining patient risk classification and generating individualized care plans. Because the assignment module 106-2 operates using deterministic mappings and threshold-based logic, each assignment decision is traceable to specific baseline clinical scores and clinical flags, thereby supporting auditability and clinical validation.

For example, in a representative implementation, a patient presenting with moderate prolapse severity, elevated pain scores, and a constipation indicator may be processed by the assignment module 106-2 such that the tier sub-module assigns the patient to a low-to-moderate intensity exercise tier, the profile sub-module assigns a prolapse-focused therapy profile with integrated pain management, the diagnosis sub-module assigns corresponding prolapse and bowel dysfunction diagnosis codes, and the adjunct sub-module recommends additional bowel management strategies. The conditional logic sub-module may further refine these assignments by reducing exercise intensity when pain and prolapse indicators are present together, thereby ensuring a safe and clinically appropriate foundation for subsequent care plan generation.

The processing module 106 further includes a risk determination module 106-3 configured to determine a patient risk classification based on the assigned exercise tier, therapy profile, diagnosis codes, baseline clinical scores, and clinical flags generated by upstream modules. The risk determination module 106-3 operates as an integrative decision layer that evaluates multiple dimensions of patient-specific clinical data in combination, rather than in isolation, to produce a clinically meaningful and safety-oriented risk classification. The module applies deterministic logic rules and predefined combinations of factors to ensure that risk classification outcomes are consistent, reproducible, and traceable to underlying data inputs.

The risk determination module 106-3 includes a risk sub-module configured to classify patients into at least two risk categories: standard and elevated. The risk sub-module evaluates a combination of severity classifications, clinical flags, assigned exercise tiers, therapy profiles, and diagnosis codes to determine the appropriate risk category. The classification process is governed by deterministic criteria that consider both individual thresholds and multi-factor interactions. In particular, the risk sub-module is configured to classify patients into elevated risk categories based on combinations of pain severity, prolapse indicators, and mental health screening results, as well as other relevant clinical flags and severity metrics.

The risk sub-module further incorporates logic to account for interactions among different clinical domains. For example, a patient with moderate pain severity alone may not meet criteria for elevated risk; however, when moderate pain severity is combined with a depression indicator derived from a mental health screening instrument, the combined condition may satisfy a predefined rule for elevated risk classification. Similarly, the presence of prolapse indicators in conjunction with high pain severity may increase the overall risk classification due to the potential for exacerbation during therapy. The deterministic logic ensures that such combinations are consistently recognized and appropriately classified.

In addition to evaluating clinical flags and severity classifications, the risk sub-module also considers the assigned exercise tier and therapy profile as contextual factors influencing risk. For instance, assignment to a lower-intensity exercise tier may indicate underlying clinical severity that contributes to elevated risk classification. At the same time, certain therapy profiles associated with complex or multi-domain conditions may further influence risk categorization. Diagnosis codes assigned by the assignment module 106-2 are also incorporated into the risk determination process, enabling alignment between diagnostic classifications and risk assessment.

The risk determination module 106-3 is further configured to support downstream clinical safety mechanisms by generating a structured risk classification output for use by the care plan generation module 106-4, the clinical review module 106-8, and other system 100 components. For example, patients classified as elevated risk may be subject to additional clinical guardrail checks, more conservative exercise parameter selection, or mandatory review flags for clinician oversight. The risk classification may also influence the frequency of monitoring, the sensitivity of adverse event escalation thresholds, and the triggering of mid-program clinical checkpoints.

The outputs of the risk determination module 106-3 are stored in the memory module 108 and maintained as part of the auditable linkage between patient-specific input data, computed clinical scores, applied rules, and generated care plan parameters. This ensures that each risk classification decision can be traced back to specific baseline clinical scores, clinical flags, and assignment outcomes, thereby supporting regulatory compliance and clinical validation.

For example, in a representative implementation, a patient presenting with high pain severity, a prolapse indicator, and a positive depression screening result may be processed by the risk sub-module such that the combination of these factors satisfies predefined criteria for elevated risk classification. The patient may also have been assigned to a low-intensity exercise tier and a therapy profile addressing both pain and prolapse. Based on the aggregation of these inputs, the risk determination module 106-3 classifies the patient as an elevated risk. This classification is then used to inform the care plan generation module 106-4, which may generate a more conservative and closely monitored care plan, and to trigger review flags within the clinical review module 106-8, ensuring appropriate clinician oversight.

In another illustrative scenario, a patient with mild symptom severity across all instruments, absence of significant clinical flags, and assignment to a standard therapy profile may be classified as standard risk by the risk sub-module. In this case, the resulting care plan may allow for standard progression parameters and reduced need for intensive monitoring. Through such deterministic, multi-factor evaluation, the risk determination module 106-3 ensures that patient risk classification is both clinically grounded and systematically consistent throughout the overall operation of the system 100.

In certain implementations, the system further comprises a referral routing module configured to direct patients to alternative or supplemental care pathways based on predefined clinical criteria. Such pathways may include referral to in-person clinical care, specialist consultation, diagnostic evaluation, or escalation to higher-acuity treatment programs. The referral routing decisions are determined using deterministic rules based on baseline clinical scores, clinical flags, patient risk classification, adherence patterns, or insufficient therapeutic progress. This configuration enables integration of digital care delivery with conventional clinical pathways while maintaining rule-based decision transparency and traceability.

The processing module 106 further includes a care plan generation module 106-4 configured to generate an individualized pelvic floor physical therapy care plan based on the patient risk classification determined by the risk determination module 106-3, in combination with the assigned exercise tier, therapy profile, diagnosis codes, baseline clinical scores, and clinical flags. The care plan generation module 106-4 functions as a structured synthesis layer that transforms upstream clinical determinations into a parameterized, executable therapeutic program. The module operates using deterministic rule sets and predefined therapy templates to ensure that generated care plans are consistent, clinically interpretable, and reproducible while remaining personalized to patient-specific conditions and preferences.

The care plan generation module 106-4 includes a parameter sub-module configured to define therapeutic exercises with parameters. The parameter sub-module determines specific exercise parameters, including repetition counts, hold durations, relaxation durations, session frequency, and progression criteria based on the assigned exercise tier, therapy profile, and patient risk classification. Predefined parameter ranges and threshold-based rules govern the parameter definitions to ensure clinical safety and appropriateness. For example, a patient classified as elevated risk and assigned to a low-intensity exercise tier may receive exercise parameters characterized by reduced repetition counts, shorter hold durations, extended relaxation durations, and lower session frequency, whereas a patient classified as standard risk may receive progressively higher repetition counts and more frequent sessions. The progression criteria defined by the parameter sub-module specify conditions under which exercise parameters are adjusted over time, such as improvements in symptom scores or adherence levels, or the absence of adverse events.

The care plan generation module 106-4 further includes a personalization sub-module configured to personalize exercise instructions based on a received patient cue preference. The personalization sub-module incorporates cueing modalities, including biologic cueing, imaginative cueing, breath-based cueing, or contraction cueing, into the instructional content of the care plan. This personalization ensures that therapeutic instructions align with patients' comprehension, engagement, and comfort. For instance, a patient indicating a preference for imaginative cueing may receive instructions framed using visualization techniques, such as imagining lifting or drawing up pelvic floor muscles. In contrast, a patient preferring breath-based cueing may receive instructions synchronized with inhalation and exhalation patterns. The personalization sub-module applies deterministic selection rules to ensure that the selected cueing modality is consistently integrated across all exercises within the care plan.

The care plan generation module 106-4 also includes a plan assembly sub-module configured to assemble the individualized pelvic floor physical therapy care plan. The plan assembly sub-module integrates outputs from the parameter sub-module and the personalization sub-module, along with therapy profile specifications and adjunct therapy recommendations generated by the assignment module 106-2, into a structured care plan format. The assembled care plan may include a sequence of exercises, parameterized instructions, progression guidelines, and adjunct therapy components. The plan assembly sub-module further ensures that the care plan is internally consistent and aligned with the patient's risk classification, including safeguards such as conservative parameter settings for elevated-risk patients and progression pathways for standard-risk patients.

The care plan generation module 106-4 is further configured to generate care plans that support downstream processes, including documentation, clinical review, and outcome tracking. For example, the generated care plan may be formatted by the documentation module 106-9 to enable structured SOAP documentation and by the outcome collection module 106-5 to facilitate monitoring of adherence and outcome data. Additionally, the care plan may incorporate references to a clinical logic version identifier stored in the memory module 108, thereby enabling traceability of the rules and parameters used during plan generation.

The outputs of the care plan generation module 106-4 are stored in the memory module 108 and transmitted to the output interface 110 for presentation to the patient and, where applicable, to a supervising clinician. The care plan serves as the basis for treatment execution, and its parameters directly influence the collection of longitudinal outcome data, including adherence patterns, symptom progression, and adverse events.

For example, in a representative implementation, a patient classified as elevated risk with a pain-dominant therapy profile may receive a care plan generated by the care plan generation module 106-4 that includes low-intensity pelvic floor exercises with reduced repetition counts, shorter hold durations, and extended relaxation intervals. The personalization sub-module may incorporate breath-based cueing instructions to guide the patient in coordinating muscle contractions with controlled breathing. The plan assembly sub-module integrates these elements into a structured daily routine with clearly defined progression criteria, such as increasing repetitions only after a predefined period of symptom stability and adequate adherence.

In another illustrative scenario, a patient classified as standard risk with mild symptom severity may receive a care plan that includes moderate-intensity exercises with higher repetition counts and more frequent sessions, along with progression criteria that allow gradual increases in exercise intensity. If the patient has indicated a preference for imaginative cueing, the instructions may include visualization-based prompts to enhance engagement. Through such deterministic yet personalized plan construction, the care plan generation module 106-4 ensures that each individualized pelvic floor physical therapy care plan is tailored to the patient's clinical profile while maintaining consistency, safety, and alignment with predefined therapeutic frameworks.

The processing module 106 further includes an outcome collection module 106-5 configured to collect longitudinal treatment outcome data generated from execution of the individualized pelvic floor physical therapy care plan. The outcome collection module 106-5 operates as a continuous data acquisition and monitoring layer that captures patient-reported and system-derived data over time, enabling tracking of treatment effectiveness, adherence, and safety. The collected longitudinal treatment outcome data include, but are not limited to, symptom progression, treatment adherence, functional outcome measures, repeated clinical assessment results, and adverse event reports, thereby aligning with the claimed definition of outcome data.

The outcome collection module 106-5 includes an outcome sub-module 106-51 configured to capture symptom progression and repeated clinical assessment results. The outcome sub-module 106-51 is configured to re-administer a subset of baseline clinical assessment instruments at predetermined intervals, including during a mid-program clinical checkpoint, and to compute deltas relative to baseline clinical scores. These deltas represent changes in symptom severity and functional outcomes over time and are used to classify a concern level using deterministic thresholds. The outcome sub-module 106-51 further supports the generation of triggered review flags when worsening or insufficient improvement is detected. For example, a patient who initially reported moderate pain severity may be reassessed at a mid-program checkpoint. If the pain score exceeds a predefined threshold, the system 100 may classify the change as a regression and generate a review flag for clinician oversight.

The outcome collection module 106-5 further includes a daily adherence logging sub-module 106-52 configured to receive patient adherence entries and to perform rolling-window analysis. The daily adherence logging sub-module 106-52 captures patient entries that include exercise completion status and date information, thereby enabling tracking of whether prescribed therapeutic activities are performed as scheduled. The sub-module 106-52 is further configured to permit retroactive entries, which may be marked with a backfill indicator to distinguish them from real-time entries. The daily adherence logging sub-module 106-52 computes rolling adherence rates over a defined temporal window, such as a weekly or multi-day window, and evaluates adherence performance against predefined thresholds. When adherence rates fall below these thresholds, the sub-module 106-52 generates adherence-concern flags that may be used to trigger clinical review or to adjust care plan parameters. Additionally, the sub-module 106-52 supports the aggregation of adherence data at the month level or other extended time intervals to facilitate longitudinal trend analysis. For example, a patient who completes prescribed exercises on four out of seven days within a weekly window may have an adherence rate computed and compared against a predefined threshold, resulting in an adherence concern flag if the threshold is not met.

The outcome collection module 106-5 also includes a structured adverse event capture sub-module 106-53 configured to collect adverse event data with severity-gated escalation. The structured adverse event capture sub-module 106-53 captures patient-reported adverse events, including new-symptom indicators, free-text descriptions, and numeric severity scales. The sub-module 106-53 applies deterministic severity thresholds to evaluate reported events and to elevate concern levels when severity exceeds predefined limits. Upon detection of high-severity events, the sub-module 106-53 generates adverse event review flags that may be routed to the clinical review module 106-8 for further evaluation. Additionally, the structured adverse event capture sub-module 106-53 incorporates the captured adverse event data into outcome records stored in the memory module 108, thereby enabling inclusion of such data in downstream statistical analysis and biomarker generation. For example, a patient reporting a new onset of pelvic pain with a high severity rating may trigger an escalation condition, resulting in the generation of a review flag and inclusion of the event in outcome datasets used for pattern analysis.

The outcome collection module 106-5 is further configured to integrate data from the outcome sub-module 106-51, the daily adherence logging sub-module 106-52, and the structured adverse event capture sub-module 106-53 into a unified outcome record structure. This unified structure supports maintenance of an auditable linkage between patient-specific input data, computed clinical scores, applied rules, generated care plan parameters, and collected outcome data. The outcome collection module 106-5 also facilitates interaction with the regression detection module 106-11, which may utilize domain-specific deltas computed from repeated assessments to detect multi-domain regression and generate domain-specific review flags.

The outputs of the outcome collection module 106-5 are stored in the outcome module 108-4 of the memory module 108 and are made available to downstream modules, including the feedback module 106-6 and the biomarker generation module 106-7. The collected longitudinal data serve as the basis for updating baseline clinical scores, refining assignment and risk determination, and identifying statistically significant clinical patterns across patient populations.

For example, in a representative implementation, a patient executes a prescribed care plan over several weeks and logs daily adherence entries indicating completion status of prescribed exercises. The daily adherence logging sub-module 106-52 computes rolling adherence rates and identifies a decline in adherence below a predefined threshold, thereby generating an adherence concern flag. Concurrently, the patient completes a mid-program reassessment, and the outcome sub-module 106-51 computes a slight worsening in pain scores, triggering a regression flag. Additionally, the patient reports a new adverse event characterized by increased pelvic discomfort, captured by the structured adverse event capture sub-module 106-53 and evaluated against severity thresholds, resulting in escalation and the generation of an adverse event review flag. These combined outcome data points are aggregated into the patient's outcome record and subsequently used by the feedback module 106-6 to adjust care plan parameters and by the clinical review module 106-8 to prompt clinician intervention, thereby supporting a closed-loop and safety-aware treatment process.

In certain scenarios, the longitudinal treatment outcome data collected by the outcome collection module 106-5 are structured for deterministic reuse within the system 100 rather than serving as direct inputs to autonomous model retraining. The captured adherence data, adverse event data, symptom progression, and reassessment results are stored in association with clinical logic version identifiers. They are subsequently evaluated using predefined statistical and rule-based frameworks. This ensures that outcome-driven refinements to the system occur through controlled updates to deterministic logic rather than through unsupervised or continuous learning mechanisms.

In certain implementations, the system further comprises an engagement module configured to initiate patient-specific interventions, notifications, reminders, or educational content delivery in response to detected adherence patterns, adherence threshold violations, or changes in patient engagement metrics. The engagement module operates based on deterministic rules and predefined trigger conditions derived from adherence data and longitudinal outcome patterns. In this manner, the engagement module may adjust communication frequency, content type, or intervention strategy in a rule-governed manner to improve patient compliance, reinforce adherence behavior, and support improved therapeutic outcomes.

The processing module 106 further includes a feedback module 106-6 configured to update one or more baseline clinical scores, clinical flags, exercise tier, therapy profile, diagnosis codes, and patient risk classification based on longitudinal treatment outcome data collected during execution of the individualized care plan. The feedback module 106-6 operates as a closed-loop adaptation layer that incorporates newly collected outcome data into the system 100's decision framework, thereby enabling iterative refinement of clinical determinations and subsequent care plan generation. The feedback module 106-6 ensures that updates are performed using deterministic rules and statistically supported evaluations so that modifications remain consistent, traceable, and clinically interpretable.

The feedback module 106-6 includes an update sub-module configured to perform rule-based updates to system 100 parameters and patient-specific classifications. The update sub-module receives longitudinal treatment outcome data from the outcome collection module 106-5, including symptom progression, adherence patterns, functional outcome measures, repeated clinical assessment results, and adverse event reports. Based on this data, the update sub-module recalculates or adjusts baseline clinical scores by incorporating newly observed values or computed deltas relative to baseline measurements. The update sub-module further updates clinical flags by re-evaluating threshold conditions using the most recent outcome data, thereby enabling dynamic activation or deactivation of indicators such as pain severity, depression, prolapse, or constipation.

In addition to updating scores and flags, the update sub-module is configured to modify assignment-related outputs, including exercise tier, therapy profile, and diagnosis codes. These updates are performed using deterministic logic rules that consider changes in severity classifications, adherence performance, and safety indicators. For example, if longitudinal data indicate sustained improvement in symptom severity and consistent adherence above a predefined threshold, the update sub-module may reassign the patient to a higher exercise tier with increased therapeutic intensity. Conversely, detection of worsening symptoms, reduced adherence, or adverse events may result in reassignment to a lower exercise tier, modification of the therapy profile, or prioritization of different diagnosis codes to reflect the updated clinical state.

The update sub-module also updates the patient risk classification by re-evaluating the combination of updated clinical scores, clinical flags, assignment outputs, and outcome-derived indicators. For instance, a patient initially classified as elevated risk may be reclassified as standard risk following sustained improvement in pain severity and resolution of associated clinical flags, while a patient exhibiting regression or adverse events may be escalated to an elevated risk classification. These updates ensure that the system 100 maintains an accurate and current representation of patient risk throughout the treatment lifecycle.

The feedback module 106-6 further includes a statistical sub-module configured to perform statistical analysis on aggregated and patient-specific outcome data. The statistical sub-module computes descriptive statistics to summarize trends in symptom progression, adherence rates, and functional outcomes. In addition, the statistical sub-module is configured to compute effect sizes using Cohen's d to quantify the magnitude of changes between baseline and follow-up measurements, thereby providing a standardized measure of treatment impact. The statistical sub-module also performs statistical significance testing using Welch's t-test to assess whether observed changes in clinical scores or outcomes are statistically significant, particularly when sample sizes or variances differ across measurement intervals or patient subgroups.

The outputs of the statistical sub-module are used by the update sub-module to inform decision-making processes, ensuring that updates to scores, flags, assignments, and risk classifications are supported not only by deterministic thresholds but also by statistically validated evidence. For example, a statistically significant reduction in pain scores with a large effect size may trigger an upward adjustment in exercise tier and progression criteria. In contrast, non-significant changes may result in maintenance of the current care plan parameters.

The feedback module 106-6 is further configured to interact with the biomarker generation module 106-7 by incorporating statistically derived insights and detected patterns into future updates. Additionally, updates performed by the feedback module 106-6 are stored in the memory module 108 along with corresponding clinical logic version identifiers, thereby maintaining an auditable linkage between input data, applied rules, statistical analyses, and resulting decision parameters.

For example, in a representative implementation, a patient undergoing therapy shows a gradual reduction in symptom severity across repeated assessments, with adherence rates consistently exceeding predefined thresholds. The statistical sub-module reports a significant reduction in pain scores with a positive effect size (Cohen's d) and confirms statistical significance using Welch's t-test. Based on these results, the update sub-module updates the baseline clinical scores to reflect improved status, removes certain clinical flags, reassigns the patient to a higher exercise tier, and updates the therapy profile to incorporate more advanced exercises. The patient risk classification is also adjusted from elevated risk to standard risk, reflecting the improved clinical condition.

The feedback module 106-6 operates within a bounded deterministic update framework in which statistical outputs, including effect sizes and significance measures, inform clinician-defined or administratively approved modifications to system parameters. The system 100 thereby avoids real-time autonomous modification of care plans based solely on statistical correlations, and instead utilizes statistically derived insights to guide controlled updates to scoring functions, assignment criteria, and therapy configurations. This preserves the integrity of the deterministic architecture while still enabling evidence-based refinement over time.

In another illustrative scenario, a patient exhibits declining adherence and reports increasingly severe adverse events. The statistical sub-module may identify a lack of statistically significant improvement or a worsening trend in symptom scores. In response, the update sub-module updates clinical flags to reflect increased risk, lowers the assigned exercise tier, modifies the therapy profile to a more conservative approach, and elevates the patient risk classification. These updates are then used to generate a revised care plan and to trigger additional clinical review, thereby ensuring that the system 100 continuously adapts to patient-specific outcomes while maintaining safety and effectiveness.

The processing module 106 further includes a biomarker generation module 106-7 configured to partition patients into subgroups and generate digital biomarkers by applying statistical analysis to aggregated clinical, therapy, and longitudinal outcome data. The biomarker generation module 106-7 operates on datasets stored in the memory module 108, including baseline clinical scores, behavioral attributes, therapy parameters, adherence data, and outcome measures. The biomarker generation module 106-7 includes a partition sub-module configured to segment patients into subgroups based on combinations of baseline clinical scores, behavioral attributes, and therapy parameters. These partitions may be defined using deterministic grouping criteria, such as severity ranges, presence of specific clinical flags, adherence patterns, or assigned therapy profiles. For example, patients with moderate pain severity, high adherence, and a specific therapy profile may be grouped into a common subgroup for analysis.

The biomarker generation module 106-7 further includes an analysis sub-module configured to apply statistical analysis to each subgroup to detect statistically significant clinical patterns and generate digital biomarkers. The analysis sub-module may utilize outputs from the statistical sub-module, including descriptive statistics, effect sizes using Cohen's d, and statistical significance testing using Welch's t-test, to identify patterns of treatment response or outcome variability. The generated digital biomarkers represent statistically supported associations between patient characteristics, therapy parameters, and clinical outcomes, and may be used to inform future assignment, risk determination, and care plan generation. For example, the analysis sub-module may identify that patients in a particular subgroup exhibit statistically significant improvement under a specific set of exercise parameters, thereby generating a biomarker to guide subsequent therapy optimization.

The system 100 maintains persistent records of inputs, applied rules, intermediate computations, and generated outputs to enable auditability and traceability. Each generated care plan or decision output may be associated with a corresponding rule execution trace that identifies the specific rules, thresholds, and inputs that contributed to the result. These records may be stored in structured databases and retrieved for validation, compliance, or analysis.

In certain implementations, analytical techniques beyond deterministic rule execution may be applied within limited portions of the system associated with statistical evaluation of aggregated outcome data, including within the biomarker generation processes and feedback analysis layers. Such techniques may include statistical modeling, comparative analysis, or cohort-based pattern detection applied to longitudinal outcome records. However, the core operational modules responsible for care plan generation, including the deterministic scoring module, assignment module, risk determination module, and care plan generation module, continue to operate exclusively using predefined deterministic scoring functions, rule-based logic, and threshold-driven decision pathways. Any insights derived from statistical or analytical processes are incorporated into the system only through controlled updates to deterministic rules, thresholds, or mappings, thereby preserving reproducibility, auditability, and consistent execution of clinical decision logic.

The operations described herein may be performed in real-time, near-real-time, or batch-processing modes, depending on the configuration of system 100. In certain scenarios, portions of the processing pipeline are triggered dynamically based on user interaction, data availability, or predefined schedules.

Accordingly, the system 100 provides an end-to-end deterministic processing pipeline that transforms structured input data into clinically actionable outputs while maintaining traceability, reproducibility, and adaptability across different application domains. The digital biomarkers generated by the biomarker generation module 106-7 are not directly executed as autonomous decision-making agents but are instead incorporated into the deterministic framework as candidate refinements subject to validation, threshold definition, and controlled deployment. This distinguishes the system 100 from predictive analytics platforms that directly operationalize statistical associations, as all biomarker-derived insights are translated into explicit rule modifications prior to influencing care plan generation.

In contrast to conventional systems that independently perform intake, scoring, intervention assignment, or outcome analysis, the present system integrates deterministic forward clinical decision generation with a feedback-driven statistical refinement loop. Outcome-derived biomarkers are systematically incorporated into subsequent deterministic rule execution, thereby forming a closed-loop decision architecture that continuously improves the relevance of interventions while preserving deterministic traceability and reproducibility.

The processing module 106 further includes a clinical review module 106-8 configured to enforce clinical guardrail checks and generate review flags to support clinician oversight. The clinical review module 106-8 includes a guardrail sub-module configured to enforce predefined clinical guardrail checks, including verification of minimum review duration, red-flag screening, and clinical documentation requirements. These guardrail checks ensure that care plan modifications and clinical decisions adhere to safety and regulatory constraints. The clinical review module 106-8 further includes a flag sub-module configured to generate review flags, including always-review flags and triggered flags. Always-review flags are deterministically generated at care plan creation based on baseline instrument scores and risk factors, while triggered flags are dynamically generated during post-treatment monitoring based on longitudinal score changes, adherence patterns, or adverse event reports. For example, a patient classified as an elevated risk at baseline may automatically receive an always-review flag. At the same time, a sudden decline in adherence or a worsening of symptom scores may trigger a flag requiring clinician intervention.

The processing module 106 further includes a documentation module 106-9 configured to generate structured SOAP (Subjective, Objective, Assessment, Plan) clinical encounter documentation from the individualized care plan and associated patient data. The documentation module 106-9 includes a documentation sub-module configured to compile patient-reported data, computed clinical scores, assigned therapy parameters, and outcome measures into a standardized SOAP format. The generated documentation supports clinical recordkeeping, auditability, and regulatory compliance. For example, subjective data may include patient-reported symptoms and adherence feedback, objective data may include computed scores and assessment results, assessment may summarize clinical interpretation, and plan may include the generated therapeutic regimen.

The processing module 106 further includes a protocol modification module 106-10 configured to modify therapeutic protocols by substituting one or more therapeutic actions with alternative actions upon detection of a predefined condition while preserving associated parameter values. The protocol modification module 106-10 includes a substitution sub-module configured to identify conditions such as adverse events, contraindications, or patient-specific limitations, and to replace affected therapeutic actions with alternative actions that maintain equivalent parameter structures, including repetition counts, durations, and frequency. This ensures continuity of therapy while adapting to patient safety requirements. For example, if a specific pelvic floor contraction exercise is associated with discomfort or adverse events, the substitution sub-module may replace it with a functionally similar exercise while preserving the prescribed repetition and duration parameters.

The processing module 106 further includes a regression detection module 106-11 configured to detect multi-domain regression during post-treatment checkpoints. The regression detection module 106-11 includes a domain sub-module configured to compute domain-specific deltas independently for a plurality of clinical instruments, including changes in pain, bowel function, prolapse, and mental health scores relative to baseline or prior measurements. The regression detection module 106-11 further includes a threshold sub-module configured to apply worsening thresholds for each domain and to generate domain-specific review flags when regression exceeds the respective thresholds. This multi-domain approach ensures that deterioration in any clinical domain is detected and addressed independently. For example, a patient may show improvement in pain scores but worsening in bowel function scores; the regression detection module 106-11 identifies the domain-specific regression and generates a corresponding review flag.

The processing module 106 further includes an agreement analysis module 106-12 configured to compute an AI-clinician agreement rate from aggregated outcome records and to present the agreement rate as a regulatory compliance metric. The agreement analysis module 106-12 includes an agreement sub-module configured to compare a number of care plans approved without clinician modifications to the total number of completed care plans. Based on this comparison, the agreement sub-module computes an agreement rate reflecting the level of concordance between system-generated and clinician-approved care plans. This metric may be used for regulatory reporting and quality assurance. For example, if a high proportion of care plans are approved without modification by clinicians, the computed agreement rate may indicate strong alignment between automated recommendations and clinical judgment, supporting confidence in the system's performance. Together, the biomarker generation module 106-7, clinical review module 106-8, documentation module 106-9, protocol modification module 106-10, regression detection module 106-11, and agreement analysis module 106-12 provide advanced analytical, safety, compliance, and optimization capabilities that enhance the robustness, adaptability, and clinical reliability of the system 100.

In certain implementations, the agreement analysis module 106-12 operates independently of any specific therapeutic domain and applies to a broad class of clinical decision support environments in which automated outputs are subject to clinician validation, approval, or modification. The computed agreement rate may be utilized as a quantitative performance metric for regulatory reporting, audit processes, certification within controlled evaluation environments, and validation of system reliability. The agreement rate may further be tracked longitudinally across clinical logic versions to evaluate improvements in deterministic decision accuracy, consistency, and alignment with clinician judgment over time.

The system 100 further includes a memory module 108 configured to store, organize, and maintain all data utilized and generated by the system 100 across the full lifecycle of patient evaluation, care plan generation, treatment execution, and feedback-driven updates. The memory module 108 functions as a centralized data repository that supports persistence, traceability, auditability, and version control, thereby enabling consistent operation of all modules within the processing module 106 and ensuring compliance with clinical and regulatory requirements. The memory module 108 is structured into multiple coordinated modules, including a data module 108-1, a score module 108-2, a plan module 108-3, an outcome module 108-4, a version module 108-5, and an audit module 108-6.

The data module 108-1 is configured to store patient-specific clinical data received via the input interface 102, including responses to the plurality of validated clinical assessment instruments and other patient-provided information. The stored data may include raw instrument responses, demographic data, behavioral attributes, and patient preferences such as cueing preferences. The data module 108-1 supports the retrieval of patient-specific input data by the deterministic scoring module 106-1 and other downstream modules for processing and analysis. For example, responses to a pain instrument, bowel function instrument, and mental health screening instrument may be stored in structured form and subsequently accessed for score computation and classification.

The processing module 106 is further configured to enforce deterministic execution boundaries across all modules such that conditional logic, threshold evaluation, and rule application are performed without stochastic variation or model-based prediction. Unlike systems that dynamically alter care pathways using continuously retrained models, the system 100 maintains version-controlled clinical logic definitions, wherein any modification to scoring rules, assignment thresholds, or therapy parameters is explicitly recorded and applied prospectively. This approach ensures that all clinical decisions remain explainable, reproducible, and anchored to predefined clinical criteria rather than adaptive or opaque optimization processes.

The system 100 operates using a strictly deterministic clinical decision architecture in which each output is generated through predefined rule sets, validated instrument mappings, and threshold-based logic. In contrast, the generalized digital health platforms rely on adaptive coaching frameworks, asynchronous clinician messaging, or probabilistic recommendation engines. The system 100 does not rely on black-box machine learning inference for primary care plan generation; instead, all clinical scores, flags, assignments, and care plan parameters are reproducibly derived from structured input data using transparent and auditable computational pathways. This deterministic configuration ensures that identical patient-specific clinical data consistently produce identical baseline clinical scores, clinical flags, assignments, and care plan outputs, thereby enabling regulatory traceability, clinical validation, and repeatable therapeutic decision-making.

The memory module 108 further includes a score module 108-2 configured to store computed baseline clinical scores, composite scores, severity classifications, and clinical flags generated by the deterministic scoring module 106-1. The score module 108-2 maintains both initial baseline values and updated values generated by the feedback module 106-6, thereby enabling longitudinal tracking of score evolution. This module supports consistent access to current and historical scoring data by the assignment module 106-2, risk determination module 106-3, and other components that rely on clinical score information. For instance, a composite pain score and its associated severity classification may be stored with corresponding timestamps to enable comparisons across multiple assessment intervals.

The memory module 108 also includes a plan module 108-3 configured to store individualized pelvic floor physical therapy care plans generated by the care plan generation module 106-4. The plan module 108-3 maintains structured representations of care plans, including exercise parameters, personalization elements, progression criteria, and recommendations for adjunct therapies. Each stored care plan may be associated with metadata such as creation time, patient identifiers, and links to the underlying clinical data used to generate the plan. For example, a care plan specifying repetition counts, hold durations, relaxation intervals, and cueing instructions may be stored in a structured format that enables retrieval for execution, modification, or review.

The memory module 108 further includes an outcome module 108-4 configured to store longitudinal treatment outcome data collected by the outcome collection module 106-5. The outcome module 108-4 maintains records of symptom progression, repeated clinical assessment results, adherence data, and adverse event data. This module supports temporal analysis of patient progress and provides input data for the feedback module 106-6 and the biomarker generation module 106-7. For example, daily adherence logs, rolling adherence rates, mid-program reassessment scores, and structured adverse event reports may be stored and indexed for subsequent analysis.

The memory module 108 also includes a version module 108-5 configured to store clinical logic version identifiers associated with generated care plans, scoring rules, assignment thresholds, diagnosis mappings, and therapy parameters. The version module 108-5 enables tracking of changes to system 100 logic over time by associating each generated care plan with its corresponding outcome record and a specific version identifier. When updates are made to scoring functions, assignment rules, or therapy parameters, the version module 108-5 records a new version identifier, thereby preserving historical context for previously generated outputs. For example, a care plan generated under a specific set of scoring thresholds and therapy parameter rules may be tagged with a corresponding version identifier, allowing subsequent analysis to account for the logic in effect at the time of generation.

The memory module 108 further includes an audit module 108-6 configured to maintain an auditable linkage between patient-specific input data, computed clinical scores, applied rules, generated care plan parameters, collected outcome data, and updated decision parameters. The audit module 108-6 ensures that each step of the system's 100 operation is traceable and that relationships between inputs, intermediate computations, and outputs are preserved in a structured and verifiable manner. This auditable linkage supports regulatory compliance, quality assurance, and clinical validation by enabling reconstruction of decision pathways. For example, the audit module 108-6 may record that a specific care plan was generated based on defined baseline clinical scores, associated clinical flags, applied assignment rules, and a particular version of clinical logic, along with subsequent updates driven by outcome data.

The audit module 108-6 further ensures that every deterministic decision pathway, including input data, computed scores, applied rules, generated outputs, and subsequent updates, is persistently recorded in a traceable format. This level of auditability exceeds that of conventional digital therapeutic platforms by enabling retrospective reconstruction of every care plan decision based on the exact rule set and data state present at the time of execution.

In operation, the memory module 108 enables seamless data flow between modules while preserving historical records and ensuring data integrity. For example, patient-specific input data stored in the data module 108-1 is used by the deterministic scoring module 106-1 to generate clinical scores stored in the score module 108-2. These scores inform care plan generation, with resulting plans stored in the plan module 108-3. As the patient executes the care plan, outcome data is collected and stored in the outcome module 108-4, and subsequent updates are recorded alongside version identifiers in the version module 108-5. Throughout this process, the audit module 108-6 maintains comprehensive linkage across all data elements, ensuring that each generated care plan and its evolution over time can be fully traced back to its originating inputs and governing logic.

The system 100 further includes an output interface 110 configured to present system-generated outputs to one or more users, including patients, clinicians, and administrative entities. The output interface 110 functions as a structured presentation layer that communicates the results of processing performed by the processing module 106 in a format suitable for execution, review, documentation, and analysis. The output interface 110 is operatively coupled with the memory module 108 and the processing module 106 to retrieve stored data and computed outputs, and to render such information in an interpretable and actionable manner.

The output interface 110 includes a plan output module 110-1 configured to present the individualized pelvic floor physical therapy care plan generated by the care plan generation module 106-4. The plan output module 110-1 retrieves structured care plan data from the plan module 108-3 of the memory module 108 and displays parameterized therapeutic exercises, including repetition counts, hold durations, relaxation durations, session frequency, and progression criteria. The plan output module 110-1 further presents personalization elements, such as patient cue preferences, within the care plan's instructional content. The displayed care plan may be formatted for patient comprehension and execution, including sequencing of exercises and guidance for daily or session-based routines. For example, a patient may view a care plan that specifies performing a defined number of repetitions with associated timing instructions and cue-based guidance aligned with the patient's selected cueing modality.

The output interface 110 further includes a documentation output module 110-2 configured to present structured SOAP clinical encounter documentation generated by the documentation module 106-9. The documentation output module 110-2 retrieves documentation records from the memory module 108 and displays them in a standardized format comprising subjective, objective, assessment, and plan sections. This structured presentation supports clinical recordkeeping, review, and regulatory compliance by enabling clinicians to access comprehensive documentation derived from patient-reported data, computed clinical scores, and generated care plans. For example, the documentation output module 110-2 may present a clinical encounter summary in which the subjective section includes patient-reported symptoms, the objective section includes computed clinical scores and assessment results, the assessment section provides clinical interpretation, and the plan section outlines the prescribed therapeutic regimen.

The output interface 110 also includes a review output module 110-3 configured to present review flags generated by the clinical review module 106-8, the outcome collection module 106-5, and the regression detection module 106-11. The review output module 110-3 displays both always-review flags and triggered flags, including flags associated with elevated risk classification, adherence concerns, adverse event escalation, and domain-specific regression detection. The review output module 110-3 enables clinicians to identify and prioritize cases requiring intervention, thereby supporting clinical oversight and safety. For example, a clinician may view a triggered flag indicating a significant worsening in pain scores or a high-severity adverse event, prompting further evaluation or modification of the care plan.

The output interface 110 further includes a reporting module 110-4 configured to present outcome reporting dashboards and aggregated analytical outputs. The reporting module 110-4 retrieves aggregated outcome data, statistical analysis results, and biomarker insights from the memory module 108 and the processing module 106, including outputs from the feedback module 106-6, the biomarker generation module 106-7, and the agreement analysis module 106-12. The reporting module 110-4 displays this information in structured formats suitable for monitoring treatment effectiveness, adherence trends, population-level outcomes, and regulatory metrics. For example, the reporting module 110-4 may present dashboards showing longitudinal changes in symptom scores, rolling adherence rates, statistical measures such as effect sizes, and AI-clinician agreement rates computed by the agreement analysis module 106-12.

The output interface 110 is further configured to support interaction between users and system outputs, enabling users to review, interpret, and act upon the presented information. The outputs generated and displayed through the output interface 110 are linked to corresponding data stored in the memory module 108, thereby ensuring consistency and traceability across all system operations. For example, a care plan displayed via the plan output module 110-1 may be directly associated with the clinical scores, risk classification, and version identifier stored in the memory module 108, while review flags displayed via the review output module 110-3 may be linked to specific outcome data and triggering conditions.

The modules described herein may be implemented as independent services, microservices, or integrated components within a single system 100. In some embodiments, the modules communicate via application programming interfaces (APIs) over one or more networks. The system 100 may be deployed in centralized, decentralized, or hybrid configurations, including cloud-based infrastructures, edge devices, or on-premise systems, depending on implementation requirements.

In operation, the output interface 110 enables the system 100 to communicate actionable clinical insights and treatment instructions in a structured, traceable, and transparent manner, thereby completing the end-to-end loop between data acquisition, computation, and clinical utilization. The plan output module 110-1 presents the individualized pelvic floor physical therapy care plan in a format suitable for patient execution, including parameterized therapeutic exercises, progression criteria, and personalized cueing instructions. The documentation output module 110-2 presents structured SOAP clinical encounter documentation derived from patient-specific clinical data, computed clinical scores, and generated care plans, thereby enabling clinician review and regulatory-compliant recordkeeping. The review output module 110-3 presents both always-review flags and triggered flags generated across the system 100, including those originating from baseline risk classification, adherence deficiencies, adverse event escalation, and regression detection, thereby facilitating clinician oversight and intervention. The reporting module 110-4 presents aggregated outcome reporting dashboards, statistical analyses, digital biomarkers, and AI-clinician agreement metrics, enabling monitoring of treatment effectiveness and adherence trends.

Through these coordinated presentation capabilities, the output interface 110 ensures that all outputs generated by the processing module 106 are accessible, interpretable, and directly aligned with the underlying clinical logic, stored data structures, and decision pathways maintained within the memory module 108.

In overall operation, the system 100 executes a continuous, closed-loop process for generating, monitoring, and refining personalized pelvic floor physical therapy care plans. Patient-specific clinical data, including responses to a plurality of validated clinical assessment instruments, is initially received through the input interface 102 and processed by the validation module 102-2 to enforce field-level validation and conditional instrument completion requirements, thereby preventing incomplete or inconsistent data from influencing downstream computations. The validated data is transmitted via the network module 104 and processed by the deterministic scoring module 106-1 to compute one or more baseline clinical scores, severity classifications, composite scores, and clinical flags using deterministic scoring functions. These outputs are stored in the memory module 108 and provided to the assignment module 106-2, which assigns the patient to at least one exercise tier, at least one therapy profile, one or more diagnosis codes, and any applicable adjunct therapy recommendations using predefined mappings and conditional logic.

The assigned outputs are then evaluated by the risk determination module 106-3, which determines a patient risk classification based on combinations of exercise tier, therapy profile, diagnosis codes, baseline clinical scores, and clinical flags. This risk classification is used by the care plan generation module 106-4 to generate an individualized pelvic floor physical therapy care plan comprising parameterized therapeutic exercises, personalization elements based on patient cue preferences, and structured progression criteria. The generated care plan is stored in the plan module 108-3 and presented to the patient via the output interface 110 for execution.

In some cases, the generated care plans, recommendations, and outcome metrics are presented via one or more user interfaces. These interfaces may include graphical user interfaces (GUIs), mobile applications, web-based dashboards, or clinician-facing systems. The outputs may be formatted for human readability, machine consumption, or integration with external systems, including electronic health record (EHR) platforms or third-party applications.

As the patient executes the care plan, longitudinal treatment outcome data is continuously collected by the outcome collection module 106-5, including symptom progression, repeated clinical assessment results, adherence data captured through the daily adherence logging sub-module 106-52, and adverse event data captured through the structured adverse event capture sub-module 106-53 with severity-gated escalation. This data is stored in the outcome module 108-4 and is further evaluated by the regression detection module 106-11 to identify multi-domain regression using domain-specific deltas and thresholds. Concurrently, the clinical review module 106-8 enforces clinical guardrail checks and generates both always-review flags and triggered flags based on baseline conditions and longitudinal changes, thereby ensuring ongoing safety and compliance.

The collected outcome data is then processed by the feedback module 106-6, which performs deterministic updates to baseline clinical scores, clinical flags, exercise tier, therapy profile, diagnosis codes, and patient risk classification. The feedback module 106-6 further applies statistical analysis, including descriptive statistics, effect size computation using Cohen's d, and statistical significance testing using Welch's t-test, to evaluate treatment effectiveness and inform updates. In parallel, the biomarker generation module 106-7 aggregates data across patients, partitions patients into subgroups, and generates digital biomarkers that represent statistically significant clinical patterns, which may further inform future system updates and the optimization of care plan generation.

Additional operations include the generation of structured SOAP documentation by the documentation module 106-9; substitution of therapeutic actions by the protocol modification module 106-10 upon detection of predefined conditions, while preserving parameter values; and computation of AI-clinician agreement rates by the agreement analysis module 106-12 as a regulatory compliance metric. All data, including inputs, computed scores, applied rules, care plan parameters, outcome records, and updates, are persistently stored within the memory module 108 along with associated clinical logic version identifiers maintained by the version module 108-5, while the audit module 108-6 maintains an auditable linkage across all system operations.

In an exemplary scenario, a patient submits responses to validated clinical assessment instruments via a client device, and the responses are validated and processed to compute baseline clinical scores and clinical flags. Based on these outputs, an exercise tier, therapy profile, and diagnosis codes are assigned, a patient risk classification is determined, and an individualized care plan with personalized instructions is generated. The patient follows the care plan, logs adherence data, and reports adverse events. The system monitors adherence patterns and symptom progression, detects any regression or safety concerns, and generates review flags for clinician oversight. Over time, the feedback module 106-6 updates the patient's clinical profile and adjusts the care plan accordingly, while aggregated data contributes to biomarker generation and system-wide optimization.

In another scenario, aggregated outcome data across multiple patients is analyzed to identify statistically significant improvements associated with specific therapy parameters within defined patient subgroups. These insights are incorporated into subsequent iterations of care plan generation through updated deterministic rules and version-controlled logic. Simultaneously, agreement rates between system-generated care plans and clinician approvals are computed and presented as regulatory compliance metrics. Through this integrated and iterative operational framework, the system 100 delivers a comprehensive, data-driven, and adaptive approach to personalized pelvic floor physical therapy care.

In another operational sequence, aggregated data from multiple patients are processed by the biomarker generation module 106-7 to identify statistically significant patterns, which are then incorporated into subsequent care plan generation via the feedback module 106-6. Simultaneously, the clinical review module 106-8 enforces guardrails and generates review flags for clinician oversight, while the agreement analysis module 106-12 computes agreement rates between automated care plans and clinician approvals.

For example, in one scenario, a patient submits baseline instrument responses indicating moderate incontinence and high pain severity. corresponding scores are computed, an appropriate exercise tier is assigned, and a personalized care plan with breath-based cueing is generated. Over time, the patient logs adherence data and reports a mild adverse event. Once the adverse event is detected, the concern level is escalated, and the case is flagged for clinical review. Updated outcome data is analyzed, resulting in adjusted exercise parameters and improved care plan recommendations.

In another scenario, aggregated data from multiple patients reveal that a specific therapy profile yields statistically significant improvements for patients with similar baseline characteristics. The biomarker generation module 106-7 identifies this pattern, and the feedback module 106-6 incorporates the insight into future assignment and care plan generation, thereby improving overall treatment effectiveness.

In a further scenario, regression in a subset of clinical domains is detected during a post-treatment checkpoint, triggering domain-specific review flags and prompting clinician intervention. Concurrently, the agreement analysis module 106-12 calculates that a high percentage of care plans are approved without modification, supporting regulatory compliance reporting.

Accordingly, the system 100 defines a closed-loop yet non-autonomous refinement architecture in which clinical decision-making remains rule-governed and deterministic at every stage. While longitudinal outcome data and statistical analyses contribute to system improvement, such contributions are mediated through explicit rule updates rather than continuous model adaptation. This structural distinction ensures that the system maintains clinical transparency, regulatory compliance, and reproducibility, while still benefiting from data-driven insights in a controlled and verifiable manner. In additional implementations, the system is configured for use across a plurality of clinical and non-rehabilitative domains, including, without limitation, mental health treatment planning, chronic disease management, cardiovascular rehabilitation, post-acute care coordination, and preventative care workflows. In such implementations, the plurality of validated clinical assessment instruments comprises domain-appropriate instruments including psychological screening tools, risk stratification indices, functional capacity measures, and disease-specific scoring systems. The care plan generation module correspondingly produces domain-specific outputs including therapeutic interventions, treatment recommendations, monitoring protocols, and care pathway assignments. Notwithstanding such domain variation, the system continues to operate using deterministic scoring functions, rule-based assignment logic, and feedback-driven refinement mechanisms, thereby preserving reproducibility, transparency, and auditability across all supported domains.

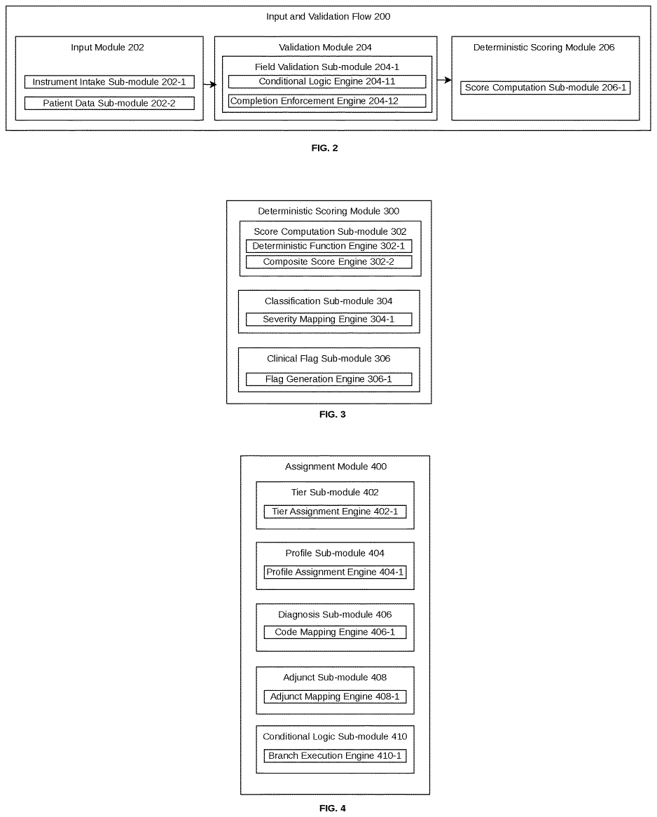

As illustrated in FIG. 1, the system 100 architecture defines the overall data flow across the system 100 components. FIG. 2 further details the input and validation processes that generate structured clinical data, which is subsequently processed by the deterministic scoring module illustrated in FIG. 3. Outputs of FIG. 3 are consumed by the intervention assignment processes of FIG. 4, which generate structured care plans. These care plans are tracked and evaluated through outcome monitoring and feedback mechanisms illustrated in subsequent figures, thereby forming a continuous and interconnected processing pipeline.

FIG. 2 illustrates a block diagram of an input and validation flow 200 of the system 100 in accordance with an exemplary embodiment of the invention. The input and validation flow 200 depicts the structured sequence through which patient-specific clinical data is received, validated, and prepared for deterministic scoring. The flow 200 includes an input module 202, a validation module 204, and a deterministic scoring module 206, each corresponding functionally to components described in the system 100 of FIG. 1 and implementing the claimed operations of receiving, validating, and processing patient-specific clinical data.

The input module 202 is configured to receive patient-specific clinical data, including responses to a plurality of validated clinical assessment instruments. The input module 202 includes an instrument intake sub-module 202-1 and a patient data sub-module 202-2. The instrument intake sub-module 202-1 is configured to receive structured responses corresponding to validated clinical assessment instruments, such as an incontinence instrument, a pain instrument, a prolapse instrument, a bowel function instrument, and a mental health screening instrument, as recited in the claims. These responses may be collected through a digital interface presented on a client device and transmitted via the network module 104 to the input module 202. The instrument intake sub-module 202-1 ensures that instrument-specific data is captured in accordance with predefined formats corresponding to each validated instrument, thereby preserving the structural integrity required for deterministic scoring.

The patient data sub-module 202-2 is configured to receive additional patient-specific data, including demographic information, behavioral attributes, and patient preferences such as cueing preferences for therapeutic instructions. This sub-module supports integration of non-instrument data with instrument-derived responses, enabling comprehensive patient profiling. For example, a patient may input age, prior medical history, and a preferred cueing modality, such as breath-based cueing or imaginative cueing, which may later be utilized by downstream modules, including the care plan generation module 106-4.

Following data intake, the validation module 204 is configured to enforce field-level validation and conditional instrument completion requirements to ensure that incomplete or inconsistent data does not influence deterministic scoring outputs, as required by the claims. The validation module 204 includes a field validation sub-module (204-1), a conditional logic engine (204-11), and a completion enforcement engine (204-12). The field validation sub-module 204-1 verifies that all required data fields are populated and conform to predefined data formats and acceptable value ranges. This includes validation of numeric ranges, categorical selections, and required responses for each instrument item.

The conditional logic engine (204-11) is configured to apply conditional validation rules based on relationships between instrument items and patient-specific responses. For example, if a patient indicates the presence of a particular symptom, the conditional logic engine 204-11 may require completion of additional instrument items associated with that symptom domain. This ensures that all necessary data required for accurate scoring and classification is collected. The conditional logic engine 204-11 thus enforces dependencies between instrument questions and dynamically adjusts validation requirements based on patient responses.

The completion enforcement engine 204-12 is configured to prevent the progression of incomplete or invalid data to downstream modules. Specifically, the completion enforcement engine 20412 ensures that all required instrument items are completed and validated before allowing the data to be processed by the deterministic scoring module 206. If incomplete data is detected, the system may prompt the user to complete missing fields or correct invalid entries. This mechanism ensures that deterministic scoring functions operate only on complete and validated datasets, thereby preserving the accuracy and reproducibility of computed clinical scores.

Upon successful validation, the patient-specific clinical data is transmitted to the deterministic scoring module 206, which corresponds to the deterministic scoring module 106-1 described in FIG. 1. The deterministic scoring module 206 includes a score computation sub-module 206-1 configured to compute one or more baseline clinical scores using deterministic scoring functions associated with each validated clinical assessment instrument. The score computation sub-module 206-1 processes the validated input data to generate structured outputs that serve as the basis for subsequent classification, flag generation, assignment, and risk determination operations.

In operation, the flow 200 ensures that patient-specific clinical data is systematically captured, rigorously validated, and deterministically processed to produce reliable baseline clinical scores. For example, a patient may submit responses to a pain instrument and a bowel function instrument through a client device. The instrument intake sub-module 202-1 captures these responses, while the patient data sub-module 202-2 records additional contextual information. The validation module 204 verifies that all required responses are complete and consistent, applying conditional logic to enforce completion of dependent questions. Only after successful validation does the system permit the deterministic scoring module 206 to compute baseline clinical scores, thereby ensuring that downstream clinical decisions are based on accurate and complete data.

Through this structured input and validation flow 200, the system enforces data integrity at the point of entry. It ensures that all subsequent processing steps, including assignment, risk determination, and care plan generation, are grounded in validated and reliable patient-specific clinical data.

FIG. 3 illustrates a block diagram of a deterministic scoring module 300 associated with the system in accordance with an exemplary embodiment of the present invention. The deterministic scoring module 300 is configured to compute one or more baseline clinical scores and one or more clinical flags from patient-specific clinical data using deterministic scoring functions, as recited in the claims. The deterministic scoring module 300 operates on validated input data received from the input interface and validation flow, producing structured outputs that serve as the foundational inputs for downstream modules, including assignment, risk determination, and care plan generation. The deterministic nature of the module ensures that identical input data yields identical outputs, thereby supporting reproducibility, auditability, and clinical transparency.

The deterministic scoring module 300 includes a score computation sub-module 302, a classification sub-module 304, and a clinical flag sub-module 306. The deterministic scoring module 300 includes the score computation sub-module 302 configured to compute baseline clinical scores associated with a plurality of validated clinical assessment instruments. The score computation sub-module 302 includes a deterministic function engine 302-1 and a composite score engine 302-2. The deterministic function engine 302-1 is configured to apply predefined scoring functions corresponding to each validated clinical assessment instrument, including but not limited to an incontinence instrument, a pain instrument, a prolapse instrument, a bowel function instrument, and a mental health screening instrument. These scoring functions are implemented in a manner that preserves the original structure and scoring methodology of each instrument, ensuring clinical validity and consistency with established assessment standards.

The deterministic function engine 302-1 processes structured instrument responses to compute individual instrument scores, which may include summation of item values, weighted scoring, or rule-based transformations as defined by the respective validated instruments. For example, responses to a pain instrument may be aggregated to produce a total pain score, while responses to a bowel function instrument may be processed to generate a bowel function score reflecting symptom severity.

The score computation sub-module 302 further includes a composite score sub-engine 302-2 configured to compute composite scores derived from multiple instrument values. The composite score engine 302-2 enables reuse of specific instrument items as components of both their original validated instrument scores and additional custom composite scores, while preserving the original instrument structure, as required by the claims. For instance, a pain-related item from a validated pain instrument may contribute both to the instrument's total score and to a composite pain score that integrates pain with functional impairment or behavioral indicators. This approach allows cross-domain scoring while maintaining the integrity of validated instruments.

The deterministic scoring module 300 further includes a classification sub-module 304 configured to compute severity classifications by mapping baseline clinical scores to predefined severity ranges. The classification sub-module 304 includes a severity mapping engine 304-1 configured to apply deterministic mapping rules that associate numeric score values with categorical severity levels, such as mild, moderate, and severe. These severity ranges are defined based on clinically established thresholds and may vary depending on the specific instrument or composite score being evaluated. The severity mapping engine 304-1 ensures consistent categorization of patient condition severity across all scoring domains. For example, a computed prolapse score may be mapped to a moderate-severity category based on predefined thresholds, which can then be used in downstream assignment and risk determination processes.

The deterministic scoring module 300 further includes a clinical flag sub-module 306 configured to generate one or more clinical flags based on the computed baseline clinical scores and underlying instrument responses. The clinical flag sub-module 306 includes a flag generation engine 306-1 configured to evaluate predefined conditions and thresholds to identify clinically relevant indicators. These clinical flags may include, but are not limited to, constipation indicators derived from bowel function responses, depression indicators derived from mental health screening results, prolapse indicators derived from prolapse instrument scores, and pain severity indicators derived from pain instrument responses, as specified in the claims.

The flag generation engine 306-1 applies deterministic logic rules to ensure that each clinical flag is directly attributable to specific input data and threshold conditions. For example, a bowel function score exceeding a predefined threshold may trigger a constipation indicator flag. In contrast, elevated responses on a mental health screening instrument may trigger a depression indicator flag. Similarly, high pain scores may trigger a pain severity indicator flag, which can influence downstream assignment and risk classification.

The outputs generated by the deterministic scoring module 300, including baseline clinical scores, composite scores, severity classifications, and clinical flags, are structured and transmitted to subsequent modules, such as the assignment module and risk determination module, and stored in the memory module for persistence and auditability. The deterministic scoring module 300 thereby establishes a consistent and traceable foundation for all downstream clinical decision-making processes within the system.

In operation, for example, a patient may complete a set of validated clinical assessment instruments, including a pain instrument and a bowel function instrument. The deterministic function engine 302-1 computes individual instrument scores, while the composite score sub-engine 302-2 generates a composite pain score incorporating selected pain-related items. The severity mapping engine 304-1 maps these scores to severity categories, such as moderate pain severity and mild bowel dysfunction. Concurrently, the flag generation engine 306-1 evaluates the scores and responses to generate clinical flags, such as a pain severity indicator and a constipation indicator. Downstream modules then use these outputs to assign therapeutic interventions and assess patient risk, ensuring that all subsequent decisions are grounded in validated, deterministically computed clinical data.

FIG. 4 illustrates a block diagram of an assignment module 400 associated with the system in accordance with an exemplary embodiment of the invention. The assignment module 400 is configured to assign a patient to at least one exercise tier, at least one therapy profile, and one or more diagnosis codes based on baseline clinical scores and clinical flags generated by the deterministic scoring module, as recited in the claims. The assignment module 400 serves as a deterministic translation layer, converting quantified clinical outputs into structured therapeutic categorizations and diagnostic mappings. The outputs of the assignment module 400 are provided to the risk determination module for subsequent patient risk classification, as indicated in FIG. 4.

The assignment module 400 includes a tier sub-module 402 configured to assign the patient to an exercise tier. The tier sub-module 402 includes a tier assignment engine 402-1 configured to apply predefined severity thresholds to baseline clinical scores and associated severity classifications to determine an appropriate exercise tier. The exercise tier represents a level of therapeutic intensity and suitability for progression and may correspond to categories such as low-, moderate-, or high-intensity tiers. The tier assignment engine 402-1 applies deterministic threshold-based logic to ensure consistent assignment. For example, a patient with high pain severity or multiple clinical flags may be assigned to a lower-intensity exercise tier. In contrast, a patient with lower severity classifications may be assigned to a higher-intensity tier.

The assignment module 400 further includes a profile sub-module 404 configured to assign the patient to at least one therapy profile. The profile sub-module 404 includes a profile assignment engine 404-1 configured to map combinations of baseline clinical scores, severity classifications, and clinical flags to predefined therapy profiles. These therapy profiles represent structured therapeutic approaches tailored to specific clinical presentations, such as pain-dominant, prolapse-focused, bowel dysfunction, or mixed-condition profiles. The profile assignment engine 404-1 applies deterministic mapping rules to ensure that patients with similar clinical characteristics are consistently assigned to the same therapy profile. For example, a patient exhibiting elevated pain severity and depression indicators may be assigned to a therapy profile emphasizing pain management and behavioral support.

The assignment module 400 also includes a diagnosis sub-module 406 configured to assign one or more diagnosis codes based on predefined mappings between clinical score ranges and diagnostic classifications. The diagnosis sub-module 406 includes a code mapping engine 406-1 configured to map baseline clinical scores, severity classifications, and instrument-specific thresholds to corresponding diagnosis codes. These diagnosis codes may align with standardized diagnostic classification regularities and are deterministically derived from quantified clinical data. For instance, a prolapse instrument score exceeding a defined threshold may result in the assignment of a prolapse-related diagnosis code, while bowel function scores indicating constipation may map to a constipation-related diagnosis code.

The assignment module 400 further includes an adjunct sub-module 408 configured to assign adjunct therapy recommendations based on clinical flags and severity thresholds. The adjunct sub-module 408 includes an adjunct mapping engine 408-1 configured to evaluate clinical flags and apply deterministic rules to determine whether additional therapeutic interventions should be recommended alongside the primary exercise-based care plan. Adjunct therapy recommendations may include behavioral strategies, relaxation techniques, or supportive interventions tailored to specific clinical indicators. For example, a depression indicator may trigger a recommendation for adjunct mental health support strategies, while a constipation indicator may result in dietary or bowel management recommendations.

The assignment module 400 also includes a conditional logic sub-module 410 configured to execute conditional logic branches that modify assignment outputs based on threshold values of baseline clinical scores or the presence of specific clinical flags. The conditional logic sub-module 410 includes a branch execution engine 410-1 configured to apply rule-based branching logic that refines or overrides initial assignments when predefined conditions are satisfied. This enables the assignment module 400 to account for interactions between multiple clinical factors rather than relying solely on independent thresholds. For example, a patient initially assigned to a moderate-intensity exercise tier based on overall severity may be reassigned to a lower-intensity tier if both a high pain severity indicator and a prolapse indicator are present. Similarly, the presence of multiple clinical flags may trigger inclusion of additional adjunct therapy recommendations or modification of the assigned therapy profile.

The outputs generated by the assignment module 400, including the assigned exercise tier, therapy profile, diagnosis codes, and adjunct therapy recommendations, are transmitted to the risk determination module for further evaluation. These outputs are also stored in the memory module to maintain traceability and support subsequent processing, including care plan generation and feedback-driven updates.

In operation, for example, a patient with moderate prolapse severity, elevated pain scores, and a constipation indicator may be processed by the assignment module 400 such that the tier assignment engine 402-1 assigns a low-to-moderate intensity exercise tier, the profile assignment engine 404-1 assigns a therapy profile addressing prolapse and pain management, the code mapping engine 406-1 assigns corresponding diagnosis codes, and the adjunct mapping engine 408-1 recommends bowel management strategies. The branch execution engine 410-1 may further refine these assignments by adjusting exercise intensity or adding additional recommendations based on combined threshold conditions. The resulting structured assignments are then provided to the risk determination module for patient risk classification, ensuring that subsequent care plan generation is aligned with the patient's clinical status.

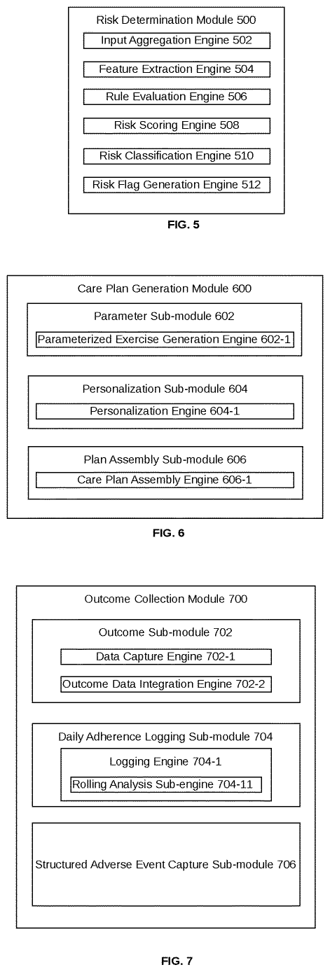

FIG. 5 illustrates a block diagram of a risk determination module 500 associated with the system in accordance with an embodiment of the present invention. The risk determination module 500 is configured to determine a patient risk classification based on the assigned exercise tier, therapy profile, diagnosis codes, baseline clinical scores, and clinical flags, as recited in the claims. The risk determination module 500 serves as an integrative evaluation component that aggregates multiple upstream outputs and applies deterministic logic to classify patients into defined risk categories, including at least standard and elevated risk. The outputs of the risk determination module 500 are utilized by downstream modules, including the care plan generation module, the clinical review module, and the output interface, to guide therapeutic intensity, monitoring requirements, and review prioritization.

The risk determination module 500 includes an input aggregation engine 502 configured to receive and consolidate inputs from upstream modules, including outputs from the deterministic scoring module and the assignment module. The input aggregation engine 502 collects baseline clinical scores, severity classifications, composite scores, clinical flags, assigned exercise tier, therapy profile, and diagnosis codes into a unified data structure for subsequent processing. This aggregation ensures that all relevant patient-specific clinical parameters are available for holistic risk evaluation. For example, the input aggregation engine 502 may combine a patient's pain severity score, prolapse indicator flag, depression indicator, assigned therapy profile, and diagnosis codes into a single structured dataset for risk analysis.

The risk determination module 500 further includes a feature extraction engine 504 configured to derive relevant analytical features from the aggregated inputs. The feature extraction engine 504 transforms raw inputs and intermediate outputs into standardized variables suitable for rule-based evaluation. These features may include normalized severity levels, the presence or absence of specific clinical flags, combinations of clinical indicators, and derived attributes representing interactions across multiple clinical domains. For instance, the feature extraction engine 504 may generate a combined indicator reflecting the co-occurrence of high pain severity and depression indicators, or a feature representing the interaction between prolapse severity and assigned exercise tier.

The risk determination module 500 further includes a rule evaluation engine 506 configured to apply deterministic rules to the extracted features to assess patient risk. The rule evaluation engine 506 implements predefined logical conditions that evaluate combinations of clinical parameters against established thresholds and criteria. These rules are structured to reflect clinically relevant risk factors, including pain severity, prolapse indicators, and mental health screening results, as specified in the claims. For example, the rule evaluation engine 506 may evaluate whether a patient exhibits both high pain severity and a depression indicator, or whether a combination of prolapse severity and functional impairment exceeds predefined thresholds indicative of elevated risk.

The risk determination module 500 further includes a risk scoring engine 508 configured to compute a quantitative or semi-quantitative risk score based on the outcomes of the rule evaluation. The risk scoring engine 508 aggregates the results of multiple evaluated rules and assigns weighted contributions to different risk factors, thereby producing an overall risk score that reflects the patient's clinical status. The scoring process is deterministic and based on predefined mappings between evaluated conditions and risk contributions. For example, the presence of multiple high-severity indicators may cumulatively increase the risk score, while the absence of such indicators may lower it.

The risk determination module 500 further includes a risk classification engine 510 configured to classify the patient into one of a plurality of predefined risk categories based on the computed risk score and evaluated rule conditions. The risk classification engine 510 applies deterministic thresholds to the risk score and/or rule outputs to assign the patient to categories such as standard risk or elevated risk, as recited in the claims. This classification may also account for specific combinations of clinical indicators, ensuring that certain high-risk conditions directly result in elevated risk classification regardless of aggregate score. For instance, a patient exhibiting high pain severity in combination with depression indicators may be directly classified as elevated risk.

The risk determination module 500 further includes a risk flag generation engine 512 configured to generate one or more risk-related flags associated with the determined risk classification. These risk flags may include indicators of elevated risk, specific contributing factors, or conditions requiring clinical review. The risk flag generation engine 512 ensures that the outputs of the risk determination module 500 are not limited to categorical classifications but also include actionable indicators for use by the clinical review module and the output interface. For example, a generated risk flag may indicate that elevated risk classification is driven by a combination of prolapse severity and mental health indicators, thereby providing context for clinician intervention.

The outputs of the risk determination module 500, including the risk score, risk classification, and associated risk flags, are transmitted to the care plan generation module to generate an individualized pelvic floor physical therapy care plan tailored to the patient's risk level. These outputs are also stored in the memory module for auditability and traceability and may be presented via the output interface to clinicians for review.

In operation, for example, a patient assigned to a moderate exercise tier and a pain-focused therapy profile, with high pain severity and a depression indicator flag, may be processed by the input aggregation engine 502 and the feature extraction engine 504 to derive relevant features that represent these conditions. The rule evaluation engine 506 evaluates these features against predefined criteria, identifying the combination of high pain severity and mental health indicators as a significant risk factor. The risk scoring engine 508 computes an elevated risk score, and the risk classification engine 510 classifies the patient as at elevated risk. The risk flag generation engine 512 then generates a risk flag indicating the contributing factors. These outputs are subsequently used to inform care plan generation, including selecting lower-intensity exercises, increasing monitoring, and potential clinician review.

Through this structured and deterministic processing framework, the risk determination module 500 ensures consistent, explainable, and clinically relevant classification of patient risk, thereby enabling downstream modules to generate safe and effective personalized pelvic floor physical therapy care plans.

FIG. 6 illustrates a block diagram of a care plan generation module 600 associated with the system in accordance with an exemplary embodiment of the present invention. The care plan generation module 600 generates an individualized pelvic floor physical therapy care plan based on a patient risk classification, as recited in the claims. The care plan generation module 600 operates as a structured synthesis component that transforms risk-stratified clinical outputs into executable therapeutic instructions. The module integrates parameterized exercise definitions, patient-specific personalization inputs, and structured plan-assembly logic to produce a comprehensive, individualized care plan suitable for patient execution and clinical review.

The care plan generation module 600 includes a parameter sub-module 602 configured to define and generate parameterized therapeutic exercises. The parameter sub-module 602 includes a parameterized exercise generation engine 602-1 configured to determine exercise parameters based on inputs received from the risk determination module, the assignment module, and baseline clinical data. The parameterized exercise generation engine 602-1 applies deterministic rules and predefined mappings to generate therapeutic exercise specifications, including repetition counts, hold durations, relaxation durations, session frequency, and progression criteria, as explicitly recited in the claims. These parameters are adjusted based on the assigned exercise tier, therapy profile, and patient risk classification, ensuring that exercise intensity and progression align with the patient's clinical status.

For example, a patient classified as elevated risk and assigned to a lower-intensity exercise tier may receive exercise parameters with reduced repetition counts, shorter hold durations, extended relaxation periods, and conservative progression criteria. Conversely, a patient classified as standard risk with lower severity scores may receive higher repetition counts and more aggressive progression criteria. The parameterized exercise generation engine 602-1 may also incorporate diagnosis codes and adjunct therapy recommendations to refine parameter selection, thereby ensuring that the generated exercises address specific clinical conditions such as pain, prolapse, or bowel dysfunction.

The care plan generation module 600 further includes a personalization sub-module 604 configured to incorporate patient-specific preferences into the care plan. The personalization sub-module 604 includes a personalization engine 604-1 configured to modify exercise instructions based on a received patient cue preference, as recited in the claims. The personalization engine 604-1 supports multiple cueing modalities, including biologic cueing, imaginative cueing, breath-based cueing, and contraction cueing. The personalization engine 604-1 adapts the instructional language and exercise presentation to align with the selected cueing modality, thereby improving patient comprehension and adherence.

For instance, a patient who prefers imaginative cueing may receive exercise instructions framed in visualization techniques, such as imagining lifting or supporting internal structures. In contrast, a patient who prefers breath-based cueing may receive instructions synchronized with inhalation and exhalation. This personalization is applied without altering the underlying exercise parameters, thereby preserving clinical consistency while enhancing usability.

The care plan generation module 600 further includes a plan assembly sub-module 606 configured to assemble the individualized care plan into a structured format suitable for presentation and execution. The plan assembly sub-module 606 includes a care plan assembly engine 606-1 configured to integrate outputs from the parameter sub-module 602 and the personalization sub-module 604 into a cohesive care plan. The care plan assembly engine 606-1 organizes therapeutic exercises into a logical sequence, incorporates progression criteria, and includes any adjunct therapy recommendations derived from the assignment module.

The care plan assembly engine 606-1 further ensures that the generated care plan is structured to support both patient execution and clinical documentation. This may include grouping exercises into sessions, defining daily or weekly schedules, and embedding metadata such as clinical logic version identifiers for traceability, as recited in the claims. The assembled care plan may also include instructions for adherence tracking and guidance for reporting adverse events, thereby facilitating integration with the outcome collection module.

The outputs generated by the care plan generation module 600 are transmitted to the memory module for storage and to the output interface for presentation to the patient and clinician. The generated care plan is also utilized by downstream modules, including the outcome collection module and the clinical review module, to monitor execution and ensure safety.

In operation, for example, a patient classified as elevated risk with a pain-focused therapy profile and a lower-intensity exercise tier may be processed by the parameterized exercise generation engine 602-1 to generate exercises with conservative parameters. The personalization engine 604-1 may adapt these exercises to incorporate breath-based cueing based on the patient's preference. The care plan assembly engine 606-1 then organizes these exercises into a structured plan, including session frequency, progression criteria, and any adjunct recommendations such as relaxation techniques. The resulting individualized pelvic floor physical therapy care plan is stored and presented to the patient for execution, ensuring that therapeutic interventions are both clinically appropriate and tailored to the patient's needs.

Through this structured and deterministic approach, the care plan generation module 600 ensures that each patient receives a personalized, parameterized, and clinically consistent pelvic floor physical therapy care plan aligned with their risk classification, clinical profile, and individual preferences.

FIG. 7 illustrates a block diagram of an outcome collection module 700 associated with the system in accordance with an exemplary embodiment of the present invention. The outcome collection module 700 is configured to collect longitudinal treatment outcome data generated from the execution of the individualized pelvic floor physical therapy care plan, as recited in the claims. The outcome collection module 700 serves as a continuous monitoring and data acquisition component that captures patient-reported and system-derived outcomes over time, thereby enabling downstream feedback, regression detection, clinical review, and biomarker generation. The collected outcome data includes, but is not limited to, symptom progression, functional outcome measures, treatment adherence data, and repeated clinical assessment results.

The outcome collection module 700 includes an outcome sub-module 702 configured to capture and integrate outcome data from multiple sources. The outcome sub-module 702 includes a data capture engine 702-1 configured to receive longitudinal inputs corresponding to patient condition and therapy execution. These inputs may be received via the input interface and transmitted through the network module from patient-operated devices or clinician interfaces. The data capture engine 702-1 collects structured data reflecting symptom progression, such as changes in pain levels or bowel function, functional outcomes such as improvements in daily activity performance, adherence data indicating completion of prescribed exercises, and reassessment results obtained through re-administration of validated clinical assessment instruments at defined intervals, including mid-program clinical checkpoints as described in the claims.

The outcome sub-module 702 further includes an outcome data integration engine 702-2 configured to consolidate and normalize the collected data into a unified longitudinal record. The outcome data integration engine 702-2 aligns data across timepoints, associates outcome data with corresponding baseline clinical scores and care plan parameters, and prepares the data for downstream processing. This integration ensures that outcome data can be compared against baseline values to compute deltas, evaluate treatment effectiveness, and support statistical analysis. For example, reassessment results collected at a mid-program checkpoint may be integrated with baseline scores to determine symptom improvement or regression.

The outcome collection module 700 further includes a daily adherence logging sub-module 704 configured to receive patient adherence entries and to perform rolling-window analysis, as recited in the claims. The daily adherence logging sub-module 704 includes a logging engine 704-1 configured to record adherence-related inputs, such as completion status and the date of care plan execution. The logging engine 704-1 supports real-time entry of adherence data as well as retroactive entries, in which previously missed entries may be recorded with a backfill marker indicating that the entry was submitted after the actual completion date. This ensures flexibility in data capture while maintaining traceability of entry timing.

The daily adherence logging sub-module 704 further includes a rolling analysis sub-engine 704-11 configured to compute adherence metrics over a defined rolling window. The rolling analysis sub-engine 704-11 calculates rolling adherence rates based on completion data within a specified time window, such as a weekly or multi-day period. It compares these rates against predefined thresholds to assess adherence performance. When adherence rates fall below defined thresholds, the rolling analysis sub-engine 704-11 generates adherence concern flags that the clinical review module may use to trigger intervention. The rolling analysis sub-engine 704-11 further supports month-level aggregation of adherence data, enabling longer-term analysis of adherence trends and patterns. For example, the system may compute a rolling adherence rate over a seven-day window and raise a flag if adherence falls below a predefined percentage threshold, while also aggregating adherence data monthly for reporting purposes.

The outcome collection module 700 further includes a structured adverse event capture sub-module 706 configured to collect adverse event data with severity-gated escalation, as recited in the claims. The structured adverse event capture sub-module 706 is configured to receive patient-reported adverse event inputs, including new-symptom indicators, free-text descriptions, and numeric severity scales. The sub-module 706 applies structured data capture protocols to ensure that adverse event information is consistently recorded and associated with corresponding care plan execution and timepoints.

The structured adverse event capture sub-module 706 further implements severity-gated escalation logic, wherein reported adverse events are evaluated against predefined severity thresholds. When a reported severity exceeds a defined threshold, the system elevates the associated concern level and generates adverse event review flags for clinician attention. The adverse event data is incorporated into outcome records maintained by outcome sub-module 702 and stored in the memory module, thereby enabling integration with downstream modules, including the clinical review module, the regression detection module, and the biomarker generation module. For example, a patient reporting a new pain symptom with a high severity rating may trigger an escalation event, resulting in the generation of a review flag and inclusion of the adverse event in longitudinal outcome analysis.

The outputs generated by the outcome collection module 700, including integrated outcome data, adherence metrics, adherence flags, and adverse event records, are transmitted to downstream modules such as the feedback module for updating clinical parameters, the regression detection module for identifying worsening conditions, and the clinical review module for enforcing guardrail checks. These outputs are also stored in the outcome module of the memory module, ensuring persistence and auditability.

In operation, for example, a patient executing an individualized care plan may log daily exercise completion through a client device. The logging engine 704-1 records completion status and dates, while the rolling analysis sub-engine 704-11 computes adherence rates over a rolling window and generates a flag if adherence falls below a threshold. Concurrently, the patient may complete periodic reassessment instruments, which are captured by the data capture engine 702-1 and integrated with baseline data by the outcome data integration engine 702-2 to assess symptom progression. If the patient reports a new symptom with a high severity rating, the structured adverse event capture sub-module 706 escalates the event and generates a review flag. All collected data is integrated into a longitudinal outcome record, enabling continuous monitoring and supporting subsequent feedback-driven updates and clinical decision-making.

Through this structured and comprehensive data collection framework, the outcome collection module 700 ensures that the system maintains an accurate, continuous, and multidimensional record of patient progress, adherence, and safety, thereby enabling adaptive, data-driven refinement of personalized pelvic floor physical therapy care plans.

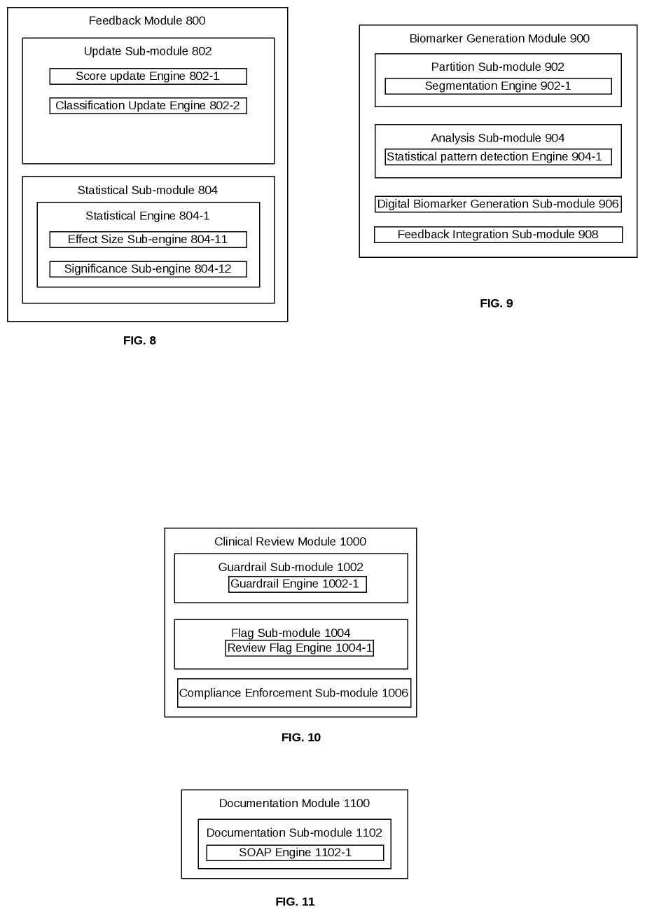

FIG. 8 illustrates a block diagram of a feedback module 800 associated with the system in accordance with an exemplary embodiment of the present invention. The feedback module 800 is configured to update baseline clinical scores, clinical flags, exercise tier, therapy profile, diagnosis codes, and patient risk classification based on longitudinal treatment outcome data for subsequent care plan generation, as recited in the claims. The feedback module 800 operates as an adaptive refinement component that incorporates outcome-driven insights into the system's deterministic framework, thereby enabling iterative improvement of patient-specific care plans while maintaining traceability and consistency.

The feedback module 800 includes an update sub-module 802 configured to perform deterministic updates to patient-specific clinical and assignment parameters based on collected outcome data. The update sub-module 802 includes a score update engine 802-1 configured to update baseline clinical scores and associated clinical flags using longitudinal treatment outcome data, including symptom progression, repeated clinical assessment results, adherence data, and adverse event records collected by the outcome collection module. The score update engine 802-1 applies predefined update rules to recompute or adjust baseline clinical scores when new assessment data is available, such as during a mid-program clinical checkpoint or post-treatment reassessment. These updates may include recalculation of instrument scores, adjustment of composite scores, and regeneration of clinical flags based on updated thresholds and input values.

In addition to updating clinical scores and flags, the score update engine 802-1 further updates assignment-related outputs, including exercise tier, therapy profile, and diagnosis codes, by reapplying deterministic assignment logic to the updated clinical data. This ensures that changes in patient condition are reflected in corresponding therapeutic categorizations. For example, improvement in pain severity scores over time may result in reassignment to a higher-intensity exercise tier or modification of the therapy profile, while worsening symptoms may trigger reassignment to a lower-intensity tier or addition of adjunct therapy recommendations.

The update sub-module 802 further includes a classification update engine 802-2 configured to update the patient risk classification based on the updated clinical scores, flags, and assignment outputs. The classification update engine 802-2 applies deterministic risk classification rules, consistent with those used by the risk determination module, to ensure that the patient's risk status reflects the most current clinical data. For instance, a reduction in symptom severity and absence of high-risk indicators may result in reclassification from elevated risk to standard risk. In contrast, the emergence of new adverse events or regression in clinical scores may result in escalation to an elevated risk classification.

The feedback module 800 further includes a statistical sub-module 804 configured to perform statistical analysis on aggregated outcome data, as recited in the claims. The statistical sub-module 804 includes a statistical engine 804-1 configured to compute descriptive statistics and evaluate treatment effectiveness across timepoints and patient populations. The statistical engine 804-1 processes outcome data stored in the memory module to compute measures such as means, variances, and changes in clinical scores over time, thereby providing quantitative insights into patient progress.

The statistical sub-module 804 further includes an effect size sub-engine 804-11 configured to compute effect sizes using Cohen's d, thereby quantifying the magnitude of change between baseline and follow-up measurements. This enables assessment of the clinical significance of observed changes beyond simple score differences. For example, the effect size sub-engine 804-11 may compute Cohen's d for improvements in pain scores between baseline and post-treatment assessments to evaluate the strength of therapeutic impact.

The statistical sub-module 804 also includes a significance sub-engine 804-12 configured to perform statistical significance testing using Welch's t-test, as recited in the claims. The significance sub-engine 804-12 evaluates whether observed differences in clinical scores or outcome measures are statistically significant, particularly when variances are unequal or sample sizes differ. This supports robust analysis of treatment outcomes across patient subgroups and timepoints.

The biomarker generation module may utilize outputs from the statistical sub-module 804 to identify statistically significant clinical patterns and from the reporting module to present outcome analytics. Additionally, statistical insights may inform refinement of deterministic rules, thresholds, and mappings used throughout the system, with updates tracked through the version module to maintain traceability.

The outputs of the feedback module 800, including updated clinical scores, clinical flags, assignment outputs, patient risk classification, and statistical analysis results, are stored in the memory module and propagated to downstream modules for subsequent care plan generation. The feedback process ensures that each iteration of care plan generation reflects the most current patient data and system insights.

In operation, for example, a patient undergoing treatment may demonstrate improvement in symptom scores over a defined period, as captured by the outcome collection module. The score update engine 802-1 recalculates baseline clinical scores based on reassessment results and updates associated with clinical flags. The classification update engine 802-2 re-evaluates the patient's risk classification, potentially reclassifying the patient from elevated risk to standard risk. The assignment-related parameters, including exercise tier and therapy profile, are correspondingly updated to reflect improved clinical status. Concurrently, the statistical engine 804-1 computes descriptive statistics on the patient's progress, the effect size sub-engine 804-11 quantifies the magnitude of improvement using Cohen's d, and the significance sub-engine 804-12 evaluates statistical significance using Welch's t-test. These outputs collectively inform subsequent care plan generation and contribute to system-wide analytical insights.

Through this structured update and analysis framework, the feedback module 800 enables the system to operate as a continuously adaptive and data-driven platform, ensuring that personalized pelvic floor physical therapy care plans evolve in response to real-world patient outcomes while maintaining deterministic consistency and auditability.

FIG. 9 illustrates a block diagram of a biomarker generation module 900 associated with the system in accordance with an exemplary embodiment of the present invention. The biomarker generation module 900 is configured to generate digital biomarkers by applying statistical analysis to aggregated clinical, therapy, and outcome data, as recited in the claims. The biomarker generation module 900 serves as a population-level analytical component that identifies statistically significant clinical patterns and treatment-response relationships across patient groups and integrates these insights into the system's adaptive feedback framework.

The biomarker generation module 900 includes a partition sub-module 902 configured to partition patients into subgroups based on combinations of baseline clinical scores, behavioral attributes, and therapy parameters, as specified in the claims. The partition sub-module 902 includes a segmentation engine 902-1 configured to perform deterministic grouping of patients into clinically meaningful segments. The segmentation engine 902-1 evaluates patient-specific attributes, including baseline clinical scores generated by the deterministic scoring module, clinical flags, behavioral attributes such as adherence patterns and engagement metrics, and therapy parameters such as assigned exercise tier, therapy profile, and parameterized exercise characteristics.

The segmentation engine 902-1 applies predefined segmentation criteria to create subgroups that reflect shared clinical and behavioral characteristics. For example, one subgroup may include patients with high pain severity and low adherence rates, while another subgroup may include patients with moderate prolapse severity and high adherence. Similarly, segmentation may consider therapy parameters, such as grouping patients receiving a specific exercise intensity or progression strategy. This partition ensures that subsequent statistical analysis is performed on homogeneous groups, thereby improving the sensitivity and interpretability of detected patterns.

The biomarker generation module 900 further includes an analysis sub-module 904 configured to perform statistical pattern detection on the segmented patient groups. The analysis sub-module 904 includes a statistical pattern detection engine 904-1 configured to apply statistical analysis techniques to aggregated data within each subgroup. The statistical pattern detection engine 904-1 leverages outputs from the feedback module, including descriptive statistics, effect sizes computed using Cohen's d, and statistical significance results derived from Welch's t-test, to identify clinically meaningful patterns and relationships.

The statistical pattern detection engine 904-1 evaluates associations between therapy parameters and outcome measures, including symptom progression, functional outcomes, adherence data, and reassessment results. For example, the engine may identify that a specific exercise parameter configuration yields a statistically significant improvement in pain scores in a subgroup of patients with high baseline pain severity and consistent adherence. The analysis sub-module 904 thus enables identification of treatment-response relationships that may not be apparent at the individual patient level.

The biomarker generation module 900 further includes a digital biomarker generation sub-module 906 configured to generate digital biomarkers representing the identified treatment-response relationships. The digital biomarker generation sub-module 906 translates statistically significant patterns into structured biomarker representations that capture correlations between clinical characteristics, therapy parameters, and observed outcomes. These digital biomarkers may include indicators of predicted treatment effectiveness, response likelihood for specific patient subgroups, or associations between adherence patterns and clinical improvement.

For example, a generated digital biomarker may indicate that patients with moderate prolapse severity and high adherence to a specific exercise regimen exhibit a statistically significant improvement in functional outcomes, with a defined effect size and significance level. Another biomarker may capture the relationship between depression indicators and reduced response to certain therapy profiles, thereby informing future care plan adjustments.

The biomarker generation module 900 further includes a feedback integration engine 908 configured to integrate generated biomarker insights into the feedback module. The feedback integration engine 908 provides the digital biomarkers as inputs to the feedback module, enabling refinement of deterministic scoring rules, assignment thresholds, therapy parameter mappings, and risk classification criteria. This integration ensures that population-level insights are incorporated into individual-level decision-making processes while maintaining deterministic consistency and version-controlled updates, as recited in the claims.

The outputs of the biomarker generation module 900, including segmented patient groups, statistical analysis results, and generated digital biomarkers, are stored in the memory module and may be presented through the reporting module for analytical review. These outputs also contribute to continuous system improvement by informing updates to clinical logic and care plan generation processes.

In operation, for example, aggregated outcome data from multiple patients may be analyzed by the segmentation engine 902-1 to group patients based on baseline pain severity, adherence patterns, and assigned exercise parameters. The statistical pattern detection engine 904-1 may identify that patients within a specific subgroup exhibit statistically significant improvement in symptom scores when a particular progression criterion is applied. The digital biomarker generation sub-module 906 encodes this relationship as a digital biomarker, which is then provided to the feedback integration sub-module 908. The feedback integration sub-module 908 incorporates this insight into the feedback module, enabling future care plans for similar patients to incorporate the identified effective therapy parameters.

Through this structured and data-driven framework, the biomarker generation module 900 enables the derivation of clinically meaningful insights from aggregated data and the continuous enhancement of the personalization and effectiveness of pelvic floor physical therapy care plans.

FIG. 10 illustrates a block diagram of a clinical review module 1000 associated with the system in accordance with an exemplary embodiment of the present invention. The clinical review module 1000 is configured to enforce clinical guardrail checks and generate review flags during care plan creation and subsequent monitoring, as recited in the claims. The clinical review module 1000 serves as a safety and compliance layer, ensuring that individualized pelvic floor physical therapy care plans and related clinical processes adhere to predefined clinical standards, documentation requirements, and risk-based review protocols. The clinical review module 1000 is operatively coupled with the processing module, the outcome collection module, and the output interface to monitor both baseline conditions and longitudinal patient data.

The clinical review module 1000 includes a guardrail sub-module 1002 configured to enforce clinical guardrail checks during care plan generation and modification. The guardrail sub-module 1002 includes a guardrail engine 1002-1 configured to evaluate whether predefined clinical requirements have been satisfied before finalizing or modifying a care plan. The guardrail engine 1002-1 applies deterministic verification rules that ensure adherence to clinical safety and documentation standards.

The guardrail engine 1002-1 performs minimum review duration verification to ensure that a clinician has engaged with the care plan for at least a predefined duration before approval or modification. This prevents premature approval of care plans without adequate clinical review. The guardrail engine 1002-1 further performs red flag screening verification by confirming that relevant clinical flags, including those generated by the deterministic scoring module, the risk determination module, the outcome collection module, and the regression detection module, have been appropriately evaluated. For example, if a patient exhibits a high pain severity indicator or a high-severity adverse event, the guardrail engine 1002-1 ensures that such conditions have been acknowledged and addressed before care plan finalization.

In addition, the guardrail engine 1002-1 performs documentation completeness verification by ensuring that all required clinical documentation elements, including structured SOAP documentation generated by the documentation module, are present and complete. This verification ensures compliance with clinical documentation standards and supports regulatory requirements. For example, the guardrail engine 1002-1 may verify that the subjective, objective, assessment, and plan sections are fully populated before allowing further progression.

The clinical review module 1000 further includes a flag sub-module 1004 configured to generate and manage review flags associated with patient conditions and care plan status. The flag sub-module 1004 includes a review flag engine 1004-1 configured to generate two categories of review flags, as specified in the claims: always-review flags and triggered flags. Always-review flags are deterministically generated at the time of care plan creation based on baseline clinical scores, risk classification, and predefined risk factors. These flags ensure that certain high-risk or clinically significant conditions are always subject to clinician review. For example, a patient classified as elevated risk based on baseline pain severity and mental health indicators may automatically generate an always-review flag.

Triggered flags are dynamically generated during longitudinal monitoring in response to changes in patient condition, adherence patterns, or adverse event reports. The review flag engine 1004-1 evaluates longitudinal outcome data collected by the outcome collection module, including adherence data, reassessment results, and adverse event inputs, to identify conditions requiring clinical attention. For instance, a decline in adherence rates below a predefined threshold, detection of multi-domain regression, or reporting of a high-severity adverse event may trigger a flag. These flags are continuously updated as new data becomes available, enabling real-time monitoring and intervention.

The clinical review module 1000 further includes a compliance enforcement sub-module 1006 configured to enforce compliance with guardrail requirements by controlling system operations related to care plan finalization and modification. The compliance enforcement sub-module 1006 prevents completion or finalization of a care plan if the guardrail checks performed by the guardrail engine 1002-1 are not satisfied. This ensures that care plans cannot be deployed without proper clinical review, red flag evaluation, and complete documentation. The compliance enforcement sub-module 1006 may also restrict modification of care plans until required review actions are completed, thereby maintaining system integrity and patient safety.

The outputs of the clinical review module 1000, including generated review flags and compliance status indicators, are transmitted to the output interface for presentation to clinicians and are stored in the memory module for auditability. These outputs may also be utilized by other modules, such as the feedback module and the agreement analysis module, to support system-wide evaluation and compliance tracking.

In operation, for example, a patient with an elevated baseline risk may generate an always-review flag during care plan creation. The guardrail engine 1002-1 verifies that the clinician has reviewed the care plan for the required duration, evaluated all relevant clinical flags, and completed necessary documentation. If any of these conditions are not satisfied, the compliance enforcement sub-module 1006 prevents finalization of the care plan. During treatment, if the patient reports decreased adherence or a high-severity adverse event, the review flag engine 1004-1 generates a triggered flag, which is presented to the clinician for intervention. The clinician may then review and modify the care plan, subject to the same guardrail checks, ensuring continuous adherence to clinical standards.

Through this structured and deterministic framework, the clinical review module 1000 ensures that all care plans and associated clinical processes are subject to rigorous oversight, thereby enhancing patient safety, supporting regulatory compliance, and maintaining the integrity.

FIG. 11 illustrates a block diagram of a documentation module 1100 associated with the system in accordance with an exemplary embodiment of the present invention. The documentation module 1100 generates structured SOAP clinical encounter documentation from the individualized pelvic floor physical therapy care plan and associated clinical data, as recited in the claims. The documentation module 1100 operates as a structured clinical reporting component that transforms patient-specific inputs, computed clinical outputs, and generated care plans into standardized documentation suitable for clinical use, recordkeeping, and regulatory compliance.

The documentation module 1100 includes a documentation sub-module 1102 configured to generate structured clinical documentation. The documentation sub-module 1102 includes a SOAP engine 1102-1 configured to automatically generate clinical encounter documentation in a structured format comprising subjective, objective, assessment, and plan sections. The SOAP engine 1102-1 integrates data from multiple system components, including the input interface, deterministic scoring module, assignment module, risk determination module, and care plan generation module, to produce a comprehensive and coherent clinical record.

The SOAP engine 1102-1 includes a subjective section generator configured to compile patient-reported information. The subjective section generator extracts patient-reported symptoms and responses to validated clinical assessment instruments received through the input interface. This includes, for example, patient-reported pain levels, bowel function symptoms, indicators of incontinence, and mental health screening responses. The subjective section generator organizes these inputs into a narrative or structured format that reflects the patient's self-reported condition at the time of assessment. This ensures that the patient's perspective and reported experiences are accurately documented during the clinical encounter.

The SOAP engine 1102-1 further includes an objective section generator configured to present measurable and computed clinical data. The objective section generator retrieves baseline clinical scores, composite scores, severity classifications, and other computed outputs generated by the deterministic scoring module. It may also include observed measures or derived metrics, such as adherence rates or functional outcome measures collected by the outcome collection module. The objective section generator structures these data points in a standardized format, ensuring that all quantifiable clinical findings are clearly documented and traceable to their underlying inputs.

The SOAP engine 1102-1 further includes an assessment section generator configured to provide clinical interpretation of the patient's condition. The assessment section generator incorporates the patient risk classification determined by the risk determination module, along with relevant clinical flags and severity indicators, to generate a structured assessment of the patient's clinical status. This section may include the interpretation of symptom severity, the identification of key contributing factors, and the classification of the patient into categories such as standard or elevated risk. The assessment section generator ensures that the clinical reasoning underlying the care plan is explicitly documented and aligned with the system's deterministic outputs.

The SOAP engine 1102-1 further includes a plan section generator configured to document the individualized pelvic floor physical therapy care plan generated by the care plan generation module. The plan section generator includes detailed descriptions of prescribed therapeutic exercises, including parameterized elements such as repetition counts, hold durations, relaxation durations, session frequency, and progression criteria, as recited in the claims. The plan section generator also incorporates any adjunct therapy recommendations assigned by the assignment module, such as behavioral strategies or supportive interventions based on clinical flags. The resulting plan section provides a complete, structured representation of the therapeutic regimen prescribed for the patient.

The documentation module 1100 is further configured to ensure that the generated documentation is consistent with system outputs and stored data. The SOAP engine 1102-1 retrieves relevant information from the memory module, including patient data, clinical scores, care plans, and outcome records, thereby ensuring that the documentation accurately reflects the patient's current clinical profile. The generated documentation may also include references to clinical logic version identifiers maintained by the version module, enabling traceability of the rules and parameters used during documentation generation.

The outputs of the documentation module 1100 are transmitted to the output interface for presentation to clinicians and may be stored in the memory module for future retrieval and audit purposes. The structured SOAP documentation supports clinical workflows, facilitates communication between care providers, and ensures compliance with documentation standards.

In operation, for example, a patient's responses to validated clinical assessment instruments are processed by the deterministic scoring module to generate baseline clinical scores and clinical flags. The risk determination module classifies the patient's risk level, and the care plan generation module produces an individualized care plan. The SOAP engine 1102-1 then compiles these inputs into a structured document, with the subjective section capturing patient-reported symptoms, the objective section presenting computed scores, the assessment section summarizing risk classification and clinical interpretation, and the plan section detailing the prescribed exercises and adjunct therapies. This documentation is then made available to clinicians through the output interface, ensuring that all relevant clinical information is organized, accessible, and aligned with the system's computational processes.

Through this structured and automated documentation framework, the documentation module 1100 ensures that all aspects of the patient's clinical encounter are comprehensively recorded, thereby enhancing clinical transparency, supporting regulatory compliance, and maintaining consistency across the system.

FIG. 12 illustrates a block diagram of a protocol modification module 1200 associated with the system in accordance with an exemplary embodiment of the present invention. The protocol modification module 1200 is configured to automatically substitute one or more therapeutic actions with alternative actions while preserving associated parameter values upon detection of a predefined condition, as recited in the claims. The protocol modification module 1200 serves as a controlled adaptation component within the system architecture, enabling the modification of individualized pelvic floor physical therapy care plans in response to specific clinical or operational triggers while maintaining consistency in therapeutic parameters and overall treatment structure.

The protocol modification module 1200 includes a condition detection engine 1202 configured to detect predefined modification triggers based on patient-specific clinical data, longitudinal outcome data, and system-generated indicators. The condition detection engine 1202 monitors inputs from multiple modules, including the outcome collection module, the deterministic scoring module, the clinical review module, and the regression detection module, to identify conditions that require modification of one or more therapeutic actions in the care plan. Such predefined conditions may include detection of adverse events exceeding severity thresholds, identification of clinical flags indicating intolerance to a particular exercise, detection of regression in one or more clinical domains, or failure to meet adherence thresholds over a defined period.

The condition detection engine 1202 applies deterministic rules to evaluate whether the detected conditions satisfy predefined criteria for protocol modification. These criteria may include threshold-based evaluations, such as severity levels associated with adverse event reports, or logical conditions derived from combinations of clinical flags and outcome measures. For example, if a patient reports increased pain severity associated with a specific therapeutic exercise, as captured by the structured adverse event capture sub-module, the condition detection engine 1202 may identify this as a trigger condition requiring substitution of that exercise.

The protocol modification module 1200 further includes a therapeutic substitution engine 1204 configured to substitute one or more therapeutic actions within the individualized care plan upon detection of a predefined condition. The therapeutic substitution engine 1204 selects alternative therapeutic actions from a predefined set of clinically appropriate substitutes mapped to the original therapeutic actions. These mappings may be based on clinical guidelines, therapy profiles, and assignment rules defined within the system. The substitution process ensures that the modified therapeutic action addresses the identified condition while remaining aligned with the patient's assigned exercise tier, therapy profile, and overall treatment objectives.

For example, if a high-intensity pelvic floor contraction exercise is associated with increased pain in a patient, the therapeutic substitution engine 1204 may replace that exercise with a lower-intensity or relaxation-focused exercise targeting the same functional domain. Similarly, if a patient demonstrates intolerance to a specific movement pattern, the engine may substitute an alternative movement that achieves comparable therapeutic outcomes without exacerbating symptoms.

The protocol modification module 1200 further includes a parameter preservation engine 1206 configured to maintain original parameter values associated with the substituted therapeutic actions. The parameter preservation engine 1206 ensures that key exercise parameters, including repetition counts, hold durations, relaxation durations, session frequency, and progression criteria, remain consistent during substitution. This preservation maintains continuity in treatment intensity and structure, thereby preventing unintended deviations in therapy dosage or progression.

The parameter preservation engine 1206 maps the original parameter values to the substituted therapeutic actions, ensuring that the modified care plan retains the intended therapeutic load and progression trajectory. For example, if an exercise prescribed with a specific number of repetitions and hold duration is replaced due to a detected condition, the parameter preservation engine 1206 applies the same repetition count and timing parameters to the alternative exercise, unless explicitly overridden by predefined safety constraints.

The outputs of the protocol modification module 1200 include updated care plan elements that reflect substituted therapeutic actions while preserving parameters. These outputs are transmitted to the care plan generation module for integration into the individualized care plan. They may also be subject to validation by the clinical review module to ensure compliance with guardrail checks. The updated care plan is stored in the memory module, with associated version identifiers updated to reflect the applied modifications, thereby maintaining traceability as recited in the claims.

In operation, for example, a patient following a prescribed care plan may report an adverse event, such as increased pain during a specific exercise. The condition detection engine 1202 identifies this input as a predefined modification trigger based on severity thresholds. The therapeutic substitution engine 1204 selects an alternative exercise that targets the same pelvic floor function but is associated with lower discomfort. The parameter preservation engine 1206 applies the original repetition count, hold duration, and session frequency to the substituted exercise, ensuring continuity in therapy dosage. The modified care plan is then updated, reviewed as needed, and presented to the patient via the output interface.

Through this structured substitution and preservation framework, the protocol modification module 1200 enables the system to adapt therapeutic interventions dynamically while maintaining deterministic control, clinical consistency, and traceability of care plan modifications.

FIG. 13 illustrates a block diagram of a regression detection module 1300 associated with the system in accordance with an exemplary embodiment of the present invention. The regression detection module 1300 is configured to detect multi-domain regression during post-treatment or mid-program clinical checkpoints, as recited in the claims. The regression detection module 1300 operates as a monitoring and analytical component that evaluates longitudinal changes in patient condition across multiple clinical domains and generates domain-specific review flags when worsening exceeds predefined thresholds. This module is operatively coupled with the outcome collection module, the memory module, and the clinical review module to ensure timely identification and escalation of clinically significant deterioration.

The regression detection module 1300 includes a domain sub-module 1302 configured to compute domain-specific deltas independently for a plurality of clinical instruments. The domain sub-module 1302 includes a domain-specific delta computation engine 1302-1 configured to compute changes in clinical scores between baseline measurements and subsequent reassessments. The domain-specific delta computation engine 1302-1 retrieves baseline clinical scores generated by the deterministic scoring module and stored in the memory module, and compares them with updated scores obtained from repeated administration of validated clinical assessment instruments, including those conducted during mid-program clinical checkpoints.

The domain-specific delta computation engine 1302-1 performs independent calculations for each clinical domain, such as pain, incontinence, prolapse, bowel function, and mental health, thereby preserving the integrity of domain-level analysis. The computed deltas represent the magnitude and direction of change in each domain, enabling identification of both improvement and deterioration. For example, an increase in pain score beyond a predefined threshold may indicate worsening pain, while a decrease in bowel function score may indicate improvement in bowel-related symptoms.

The regression detection module 1300 further includes a threshold sub-module 1304 configured to evaluate the computed domain-specific deltas against predefined worsening thresholds. The threshold sub-module 1304 includes a threshold evaluation engine 1304-1 configured to apply deterministic threshold rules for each clinical domain. These thresholds may be defined based on clinical guidelines, statistical baselines, or empirically derived criteria, and may vary across domains to reflect differences in measurement scales and clinical significance.

The threshold evaluation engine 1304-1determines whether the computed delta for each domain exceeds the corresponding worsening threshold, thereby indicating regression in that domain. For example, if a pain score increases beyond a defined threshold value, or if a mental health screening score indicates worsening depressive symptoms beyond a predefined limit, the threshold evaluation engine 1304-1 identifies these conditions as domain-specific regression events.

The regression detection module 1300 further includes a regression flag generation sub-module 1306 configured to generate domain-specific review flags when regression exceeds the respective thresholds. The regression flag generation sub-module 1306 produces structured review flags corresponding to each affected clinical domain, indicating the presence and severity of regression. These flags are transmitted to the clinical review module for further evaluation and potential intervention. They are also stored in the memory module to maintain an auditable record of detected regression events.

The regression flags generated may be integrated with other system-generated flags, including adherence-concern flags and adverse-event review flags, to provide a comprehensive view of patient status. Additionally, the regression detection outputs may be utilized by the feedback module to inform updates to clinical scores and assignments, and by the protocol modification module to trigger substitution of therapeutic actions when regression is detected.

In operation, for example, during a mid-program clinical checkpoint, a patient may complete a subset of validated clinical assessment instruments. The domain-specific delta computation engine 1302-1 calculates changes in scores relative to baseline for each domain. The threshold evaluation engine 1304-1 compares these deltas against predefined worsening thresholds and identifies that the patient's pain score has increased beyond the acceptable range. The regression flag generation sub-module 1306 generates a domain-specific review flag indicating regression in the pain domain. This flag is transmitted to the clinical review module, prompting clinician review and potential modification of the care plan.

Through this structured and domain-specific analysis framework, the regression detection module 1300 enables the system to detect clinically meaningful deterioration across multiple domains in a timely and deterministic manner, thereby supporting proactive intervention, enhancing patient safety, and ensuring adaptive refinement of personalized pelvic floor physical therapy care plans.

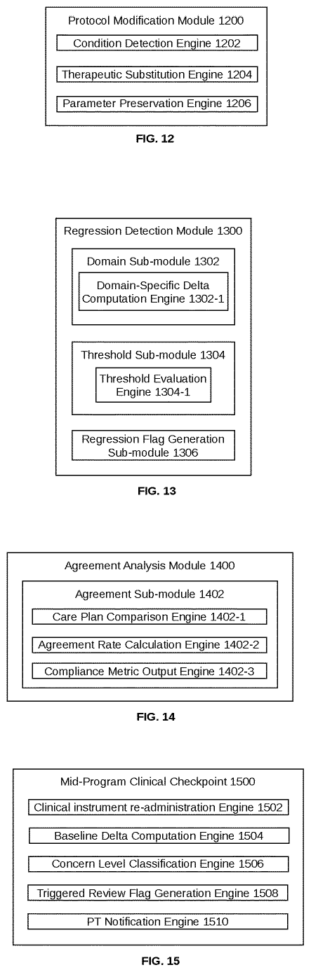

FIG. 14 illustrates a block diagram of an agreement analysis module 1400 associated with the system in accordance with an exemplary embodiment of the present invention. The agreement analysis module 1400 is configured to compute an AI-clinician agreement rate from aggregated outcome records and present it as a regulatory compliance metric, as recited in the claims. The agreement analysis module 1400 operates as a post-processing and evaluation component that quantifies the level of concordance between system-generated care plans and clinician-reviewed outcomes, thereby supporting transparency, quality assurance, and regulatory reporting.

The agreement analysis module 1400 includes an agreement sub-module 1402 configured to analyze care plan approval patterns and compute agreement metrics. The agreement sub-module 1402 includes a care plan comparison engine 1402-1 configured to compare AI-generated individualized pelvic floor physical therapy care plans with corresponding clinician-reviewed or clinician-modified care plans. The care plan comparison engine 1402-1 retrieves generated care plans from the care plan generation module and corresponding clinician interaction records, including whether the care plan was approved without modification or modified before approval, as stored in the memory module.

The care plan comparison engine 1402-1 evaluates each completed care plan instance to determine whether it was approved by a clinician without modification, indicating agreement, or modified, indicating divergence between the system-generated recommendation and clinician judgment. The comparison process may include verification of plan parameters, such as exercise tier, therapy profile, parameterized exercise details, and adjunct therapy recommendations, to determine whether substantive changes were made. For example, if a clinician accepts a generated care plan without altering exercise parameters or therapy assignments, the instance is recorded as an agreement case. In contrast, modification of exercise intensity or addition of adjunct therapies is recorded as a non-agreement case.

The agreement sub-module 1402 further includes an agreement rate calculation engine 1402-2 configured to compute an agreement rate based on aggregated data across multiple care plan instances. The agreement rate calculation engine 1402-2 determines the ratio of care plans approved without clinician modification to the total number of completed care plans within a defined dataset or time period. This computation may be expressed as a proportion or percentage and calculated for specific patient populations, therapy profiles, or time intervals to support granular analysis.

For example, the agreement rate calculation engine 1402-2 may compute that a defined percentage of care plans generated by the system were approved without modification over a specified period, thereby providing a quantitative measure of alignment between system-generated recommendations and clinician decisions.

The agreement sub-module 1402 further includes a compliance metric output engine 1402-3 configured to output the computed agreement rate as a regulatory compliance metric. The compliance metric output engine 1402-3 formats the agreement rate for presentation through the output interface and may integrate with the reporting module to include the metric in dashboards, reports, or regulatory submissions. The agreement rate serves as an indicator of system reliability, clinical acceptance, and consistency of decision-making, and may be used to demonstrate adherence to regulatory or organizational standards.

The outputs generated by the agreement analysis module 1400 are stored in the memory module. They may be linked to corresponding care plan records and outcome data, thereby maintaining an auditable record of agreement analysis. System administrators or oversight entities may also utilize these outputs to evaluate system performance and identify areas for improvement.

In operation, for example, the system may generate a series of individualized care plans for multiple patients, which clinicians subsequently review. The care plan comparison engine 1402-1 evaluates each instance to determine whether the clinician approved the plan without modification or introduced changes. The agreement rate calculation engine 1402-2 aggregates these results and computes an agreement rate representing the proportion of unmodified approvals. The compliance metric output engine 1402-3 then outputs this agreement rate to a reporting dashboard, enabling stakeholders to assess the level of alignment between automated recommendations and clinical judgment.

Through this structured comparison and computation framework, the agreement analysis module 1400 provides a quantitative measure of AI-clinician agreement, thereby supporting regulatory compliance, enhancing transparency, and enabling continuous evaluation of the system.

FIG. 15 illustrates a block diagram depicting a mid-program clinical checkpoint 1500 in accordance with an exemplary embodiment of the present invention. The mid-program clinical checkpoint 1500 is configured to re-evaluate patient condition at a predetermined interval and generate triggered review flags for a supervising clinician, as recited in the claims. The mid-program clinical checkpoint 1500 serves as an intermediate reassessment and monitoring workflow integrated into the system, enabling early detection of insufficient progress, regression, or emerging clinical concerns during the execution of the individualized pelvic floor physical therapy care plan. The checkpoint 1500 is operatively coupled with the input interface, deterministic scoring module, outcome collection module, regression detection module, and clinical review module to ensure continuity of data processing and clinical oversight.

The mid-program clinical checkpoint 1500 includes a clinical instrument re-administration engine 1502 configured to re-administer a subset of baseline clinical assessment instruments at a predefined time point, such as a mid-program interval. The clinical instrument re-administration engine 1502 selectively presents validated clinical instruments previously administered at baseline, ensuring consistent measurement and enabling direct comparison of results. The instruments may include, for example, an incontinence instrument such as ICIQ-UI SF, a pain composite instrument derived from baseline pain-related responses when baseline pain is greater than zero, and a mental health screening instrument such as PHQ-2. The clinical instrument re-administration engine 1502 ensures that only relevant instruments are re-administered based on baseline conditions, thereby reducing patient burden while preserving analytical rigor.

The mid-program clinical checkpoint 1500 further includes a baseline delta computation engine 1504 configured to compute deltas relative to baseline for each re-administered instrument. The baseline delta computation engine 1504 retrieves baseline clinical scores from the memory module and compares them with reassessment scores obtained at the checkpoint. The computation of deltas is domain-specific and may vary based on the directionality of clinical interpretation. For example, an incontinence score delta may be computed as a difference between baseline and week-four scores (ICIQ Δ=baseline−week 4), reflecting improvement when the value decreases, while a pain score delta may be computed as a difference between week-four and baseline scores (Pain Δ=week 4−baseline), reflecting worsening when the value increases. The PHQ-2 score at the checkpoint may be evaluated directly as an indicator of mental health status. These computed deltas provide quantitative measures of change in patient condition across multiple domains.

The mid-program clinical checkpoint 1500 further includes a concern level classification engine 1506 configured to classify a concern level based on the computed deltas and predefined deterministic thresholds. The concern level classification engine 1506 assigns each patient to a concern category, such as none, moderate, or high, based on the magnitude and direction of changes in clinical scores. The classification rules may incorporate thresholds specific to each clinical domain and may consider combinations of domain-level changes. For example, a patient demonstrating minimal change across all domains may be classified as having no concern; a patient exhibiting moderate worsening in pain or adherence-related indicators may be classified as having moderate concern; and a patient exhibiting significant regression or high-severity mental health indicators may be classified as having great concern.

The mid-program clinical checkpoint 1500 further includes a triggered review flag generation engine 1508 configured to generate review flags based on the classified concern level. The triggered review flag generation engine 1508 produces structured review flags that are transmitted to the clinical review module, consistent with the triggered flag category described in the claims. These flags indicate that a clinician review is required due to changes detected during the checkpoint. The flags may include metadata identifying the affected clinical domains, the magnitude of change, and the assigned concern level, thereby enabling targeted clinical intervention.

The mid-program clinical checkpoint 1500 further includes a PT notification engine 1510 configured to notify a supervising clinician or physical therapist when triggered review flags are generated. The PT notification engine 1510 communicates alerts through the output interface, ensuring timely awareness of patient status changes that may require early intervention. The notifications may include summaries of reassessment results, computed deltas, concern levels, and recommended actions, thereby facilitating efficient clinical decision-making.

The outputs generated by the mid-program clinical checkpoint 1500, including reassessment data, computed deltas, concern level classifications, and triggered review flags, are stored in the memory module and integrated into the longitudinal outcome records. The regression detection module may also use these outputs to corroborate domain-specific regression and provide feedback to update clinical scores and assignments.

In operation, for example, a patient undergoing a care plan may reach a predefined mid-program interval, such as four weeks. The clinical instrument re-administration engine 1502 presents selected instruments, including an incontinence questionnaire, a pain assessment, and a mental health screening. The baseline delta computation engine 1504 computes changes in scores relative to baseline, identifying whether the patient has improved, remained stable, or worsened in each domain. The concern-level classification engine 1506 evaluates these changes against predefined thresholds and classifies the patient as having moderate concern due to increased pain and unchanged incontinence scores. The triggered review flag generation engine 1508 generates a corresponding review flag, and the PT notification engine 1510 notifies the clinician for early intervention. The clinician may then review and modify the care plan, subject to clinical guardrail checks, thereby ensuring responsive and adaptive patient care.

Through this structured reassessment and escalation framework, the mid-program clinical checkpoint 1500 enables the system to detect and respond to clinically significant changes during treatment, thereby enhancing patient safety, improving treatment outcomes, and supporting continuous monitoring within the personalized pelvic floor physical therapy care process.

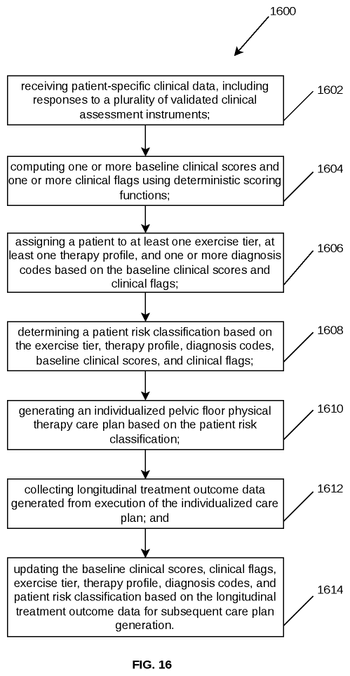

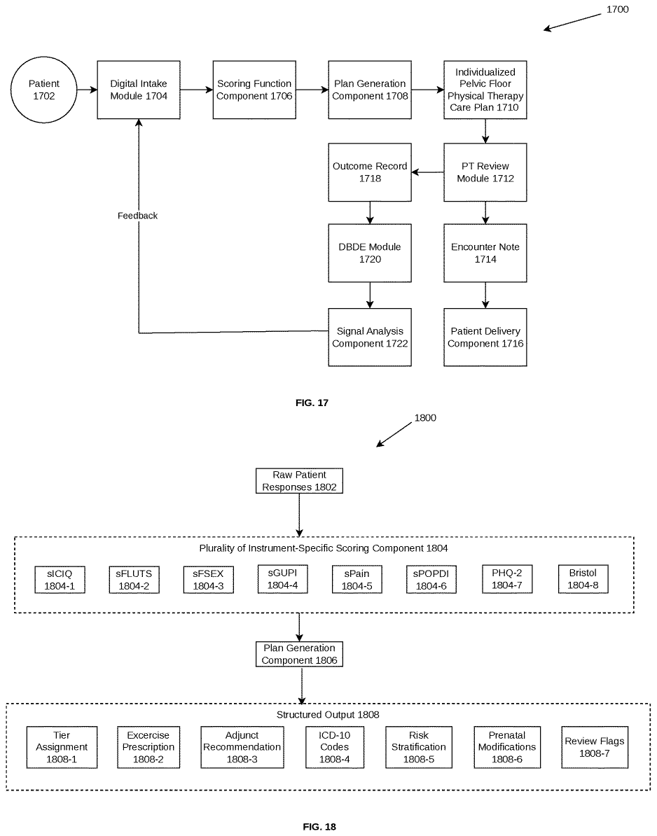

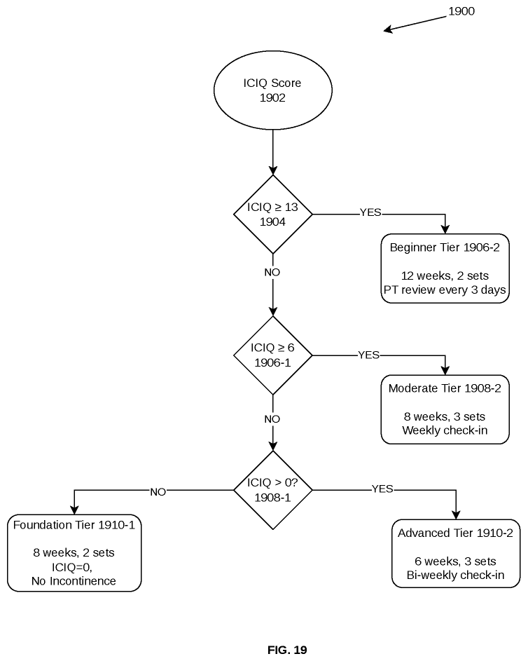

FIG. 16 illustrates a flowchart of a method 1600 for generating a personalized pelvic floor physical therapy care plan in accordance with an exemplary embodiment of the present invention. The method 1600 is executed in a sequential and iterative manner to enable continuous personalization and refinement of therapeutic interventions based on patient-specific clinical data and longitudinal treatment outcomes.

At step (a), the method includes receiving 1602 patient-specific clinical data, including responses to a plurality of validated clinical assessment instruments. The received data may include structured inputs corresponding to incontinence, pain, prolapse, bowel function, and mental health screening instruments, along with additional patient-reported information. The data is collected in a manner that ensures completeness and consistency, including enforcement of conditional instrument completion requirements to prevent incomplete or invalid responses from influencing subsequent processing. This step establishes the foundational dataset upon which all subsequent computations and decisions are based.

At step (b), the method includes computing 1604 one or more baseline clinical scores and one or more clinical flags using deterministic scoring functions. The computation involves applying predefined scoring rules to the received instrument responses to generate quantitative baseline clinical scores. These scores may further be mapped to predefined severity ranges to produce severity classifications. In addition, composite scores may be generated by combining values from multiple instruments while preserving the structure of the original instruments, such as deriving a composite pain score from individual pain-related inputs. Clinical flags are also generated to indicate specific conditions, including constipation, depression, prolapse, and pain severity. This step transforms raw patient inputs into structured clinical metrics suitable for downstream decision-making.

At step (c), the method includes assigning 1606 a patient to at least one exercise tier, at least one therapy profile, and one or more diagnosis codes based on the baseline clinical scores and clinical flags. The assignment is performed using predefined mappings and threshold-based logic. Exercise tiers are determined by severity thresholds associated with clinical scores, while therapy profiles reflect broader therapeutic categorizations aligned with the patient's condition. Diagnosis codes are assigned using mappings between clinical score ranges and diagnostic classifications. In addition, adjunct therapy recommendations may be assigned based on clinical flags and severity thresholds. Conditional logic branches may further modify these assignments based on specific combinations of scores and flags, ensuring that the resulting assignments are tailored to the patient's clinical profile.

At step (d), the method includes determining 1608 a patient risk classification based on the exercise tier, therapy profile, diagnosis codes, baseline clinical scores, and clinical flags. The risk classification assigns the patient to predefined risk levels, such as standard or elevated, using deterministic rules that consider combinations of clinical indicators. For example, elevated pain severity, presence of prolapse indicators, or mental health screening results may contribute to classification into a higher risk category. This step establishes an overall risk profile that guides the subsequent generation of the care plan.

At step (e), the method includes generating 1610 an individualized pelvic floor physical therapy care plan based on the patient risk classification. The generated care plan includes parameterized therapeutic exercises defined by repetition counts, hold durations, relaxation durations, session frequency, and progression criteria. The care plan may further incorporate personalization based on patient cue preference, including biologic cueing, imaginative cueing, breath-based cueing, or contraction cueing. The generated care plan may also include recommendations for adjunct therapy derived from earlier assignments. This step produces a structured and individualized therapeutic regimen tailored to the patient's clinical and risk profile.

At step (f), the method includes collecting 1612 longitudinal treatment outcome data generated from the execution of the individualized care plan. The collected data include symptom progression, treatment adherence, functional outcome measures, and repeated clinical assessment results. Adherence data may include daily completion status and dates, with support for retroactive entries and rolling window adherence analysis. Adverse event data may also be collected, including new-symptom indicators, free-text descriptions, and numeric severity scales, with severity-gated escalation applied when predefined thresholds are exceeded. This step ensures continuous monitoring of patient progress and treatment effectiveness over time.

At step (g), the method includes updating 1614 the baseline clinical scores, clinical flags, exercise tier, therapy profile, diagnosis codes, and patient risk classification based on the longitudinal treatment outcome data for subsequent care plan generation. The updating process involves recomputing clinical scores using new assessment data, regenerating clinical flags, and reapplying assignment and risk determination logic to reflect the patient's current condition. Statistical analysis may be performed on the outcome data, including descriptive statistics, effect size computation using Cohen's d, and statistical significance testing using Welch's t-test. The updated parameters are then used to generate refined care plans in subsequent iterations, enabling continuous personalization and improvement.

In operation, the method 1600 proceeds iteratively, wherein the updated outputs from step (g) feed back into earlier steps to support ongoing care plan generation and adjustment. For example, improvements in clinical scores may result in reassignment to a higher exercise tier and in modifications to therapy parameters. In contrast, detection of adverse events or regression may result in adjustment of therapeutic actions or escalation for clinical review. Through this continuous loop, the method 1600 enables dynamic and data-driven management of pelvic floor physical therapy care, ensuring that treatment plans remain aligned with evolving patient needs and clinical outcomes.

FIG. 17 illustrates a block diagram of an end-to-end operational flow 1700 for generating and refining a personalized pelvic floor physical therapy care plan in accordance with an exemplary embodiment of the present invention. This figure is intended as a representative workflow depiction showing how patient inputs are transformed into actionable care plans and subsequently refined through outcome-driven feedback, without limiting the scope of the broader architecture described in preceding figures.

As shown, a patient 1702 initiates the process by interacting with a digital intake module 1704, which captures patient-specific clinical data, including responses to a plurality of validated clinical assessment instruments. The collected data is provided to a scoring functions component 1706, where deterministic scoring functions are applied to compute baseline clinical scores and clinical flags. These scoring functions may include validated instruments and composite scoring mechanisms, enabling the generation of severity classifications and clinically relevant indicators. The computed outputs are then provided to a plan generation component 1708, which generates an individualized pelvic floor physical therapy care plan 1710. The generated care plan 1710 includes parameterized therapeutic exercises and may incorporate personalization features, such as cue preferences and recommendations for adjunct therapies.

Following generation, the care plan 1710 is provided to a PT review module 1712, where clinician oversight is performed in accordance with clinical guardrail requirements. This interaction results in the creation of encounter note 1714, which captures structured clinical documentation. The finalized care plan is then delivered to the patient via the patient delivery component 1716, enabling the execution of prescribed therapeutic interventions. During and after execution, longitudinal treatment outcome data are captured and stored as an outcome record 1718, including symptom progression, adherence data, and reassessment results. The outcome record 1718 is further processed by a DBDE module 1720 and a signal analysis component 1722, which perform analytical evaluation of aggregated outcome data to detect patterns and generate insights. As indicated by the feedback pathway, these insights are utilized to refine scoring rules and influence subsequent care plan generation.

In this exemplary scenario, workflow 1700 demonstrates how deterministic scoring, clinician review, and outcome-driven analytics operate in a closed-loop process to improve personalized care plans continuously. For instance, patterns identified during signal analysis may lead to refinements to scoring thresholds or composite scoring structures, thereby influencing future baseline clinical score computation and downstream assignments. This iterative refinement process enables adaptive and data-driven care plan generation while maintaining deterministic logic and traceability.

FIG. 18 illustrates a block diagram of a complete care plan generation through deterministic scoring and assignment processes 1800 in accordance with an exemplary embodiment of the present invention. This figure provides a more granular representation of how individual validated clinical assessment instruments contribute to care plan generation and is intended as an illustrative workflow without limiting the broader architecture and functionality described in the preceding figures.

As shown, the process 1800 begins with raw patient responses 1802, which represent patient-specific clinical data collected from a plurality of validated clinical assessment instruments. These responses are distributed to a plurality of instrument-specific scoring components 1804, including at least one of sICIQ 1804-1, sFLUTS 1804-2, sFSEX 1804-3, sGUPI 1804-4, sPain 1804-5, sPOPDI 1804-6, PHQ-2 1804-7, or Bristol 1804-8. In certain cases, the instrument set shown in FIG. 18 represents a subset and/or extended representation of a canonical instrument set that includes validated instruments such as ICIQ, FLUTS, GUPI, POPDI-6, PHQ-2, and Bristol. Additional instruments or derived measures, including composite pain scores or domain-specific extensions such as sexual function assessments, may be included as supplemental inputs while preserving the core deterministic scoring framework.

Each of these components 1804 corresponds to a validated instrument domain, such as incontinence, lower urinary tract symptoms, sexual function, urologic pain, general pain, and prolapse-related distress. Deterministic scoring functions are applied within each instrument pathway to compute individual instrument scores, which may include both standard instrument outputs and derived values used for composite scoring. The outputs from these instrument-specific computations are then aggregated and provided to a plan generation component 1806, which integrates the multi-domain clinical data into a unified decision framework.

The plan generation component 1806 processes the aggregated scores and clinical indicators to produce multiple structured outputs 1808 corresponding to key elements of the care plan. These standard outputs 1808 include a tier assignment 1808-1, an exercise prescription 1808-2, adjunct recommendations 1808-3, ICD-10 codes 1808-4, a risk stratification 1808-5, prenatal modifications 1808-6, and review flags 1808-7.

The tier assignment 1808-1 determines an appropriate exercise intensity level based on severity thresholds. The exercise prescription 1808-2 defines parameterized therapeutic exercises, including repetitions, hold durations, relaxation intervals, and session frequency. The adjunct recommendations 1808-3 identify additional supportive therapies based on clinical flags. The ICD-10 codes 1808-4 are assigned using predefined mappings between score ranges and diagnostic classifications. The risk stratification 1808-5 classifies the patient as standard risk or elevated risk based on combinations of clinical indicators. The prenatal modifications 1808-6 adapt therapy parameters to specific patient conditions, and the review flags 1808-7 indicate cases that require clinician oversight based on predefined criteria.

These standard outputs 1808 are subsequently integrated into a complete care plan 1810, which represents the assembled and individualized pelvic floor physical therapy program. The complete care plan 1810 consolidates all generated components into a structured and actionable format suitable for patient execution and clinician review. The integration process ensures that all assignments, prescriptions, and recommendations are consistent with one another and aligned with the patient's clinical profile derived from the initial raw responses.

In this exemplary scenario, the figure illustrates how multiple validated clinical instruments are processed in parallel and then combined using deterministic logic to produce a comprehensive care plan. For example, elevated scores on the pain and prolapse instruments may influence both risk stratification and tier assignment, as well as trigger recommendations for adjuncts and review flags. By structuring the workflow in this manner, the implementation ensures that each clinical domain contributes systematically to the final care plan, enabling a data-driven, transparent, and reproducible approach to personalized pelvic floor physical therapy.

FIG. 19 illustrates a decision flow 1900 for an exercise tier assignment in accordance with an exemplary embodiment of the present invention. The decision flow 1900 demonstrates an assignment of a patient to an exercise tier based on a baseline incontinence score, specifically an ICIQ score, in compliance with the deterministic scoring and assignment logic. This figure illustrates a rule-based branching structure that maps clinical score thresholds to predefined exercise tiers, enabling consistent, reproducible tier assignment as part of the overall care plan generation process.

As shown, the process begins with an ICIQ score of 1902 derived from patient responses to a validated incontinence instrument. The ICIQ score 1902 is evaluated through a sequence of threshold-based decision nodes. At ICIQ≥13 decision node 1904, the method determines whether the ICIQ score is greater than or equal to a first predefined threshold, such as 13. If the condition is satisfied, the patient is assigned to a beginner tier 1906-2. The beginner tier 1906-2 represents a lower-intensity therapeutic program. It may include parameters such as a defined program duration (e.g., 12 weeks), a specified number of exercise sets, and increased clinical oversight, such as periodic review intervals. This assignment reflects a higher severity classification requiring a more conservative therapeutic approach.

If the condition at decision node 1904 is not satisfied, the process proceeds to a subsequent ICIQ≥6 decision node 1906-1, where it is determined whether the ICIQ score meets or exceeds a second threshold, such as 6. If this condition is satisfied, the patient is assigned to a moderate tier 1908-2. The moderate tier 1908-2 corresponds to an intermediate level of therapy intensity. It may include defined program parameters such as an intermediate duration, a moderate number of exercise sets, and scheduled check-ins at regular intervals. This moderate tier 1908-2 reflects a moderate severity classification requiring structured but less restrictive intervention than the beginner tier.

If the condition at decision node 1906-1 is not satisfied, the process proceeds to ICIQ>0 decision node 1908-1, where it is determined whether the ICIQ score is greater than zero. If this condition is satisfied, the patient is assigned to an advanced tier 1910-2. The advanced tier 1910-2 corresponds to a higher-intensity therapeutic program and may include shorter program duration, increased exercise sets, and less frequent monitoring intervals. This assignment reflects lower symptom severity while still requiring active intervention.

If none of the threshold conditions are satisfied, indicating that the ICIQ score is equal to zero, the patient is assigned to a foundation tier 1910-1. The foundation tier 1910-1 corresponds to a baseline or maintenance-level program, which may involve minimal intervention and indicates no incontinence symptoms. This foundation tier, 1910-1, may be used for preventive or maintenance purposes rather than active rehabilitation.

In this exemplary scenario, the figure demonstrates how deterministic, threshold-based logic maps a baseline clinical score to a specific exercise tier, consistent with the assignment module's functionality. The use of predefined thresholds ensures that tier assignment is transparent, repeatable, and aligned with clinical severity classifications. Additionally, the structured progression across tiers from foundation to beginner supports scalable personalization of therapeutic intensity based on patient condition. This rule-based framework may be integrated with other clinical indicators and modules to generate a comprehensive and individualized pelvic floor physical therapy care plan.

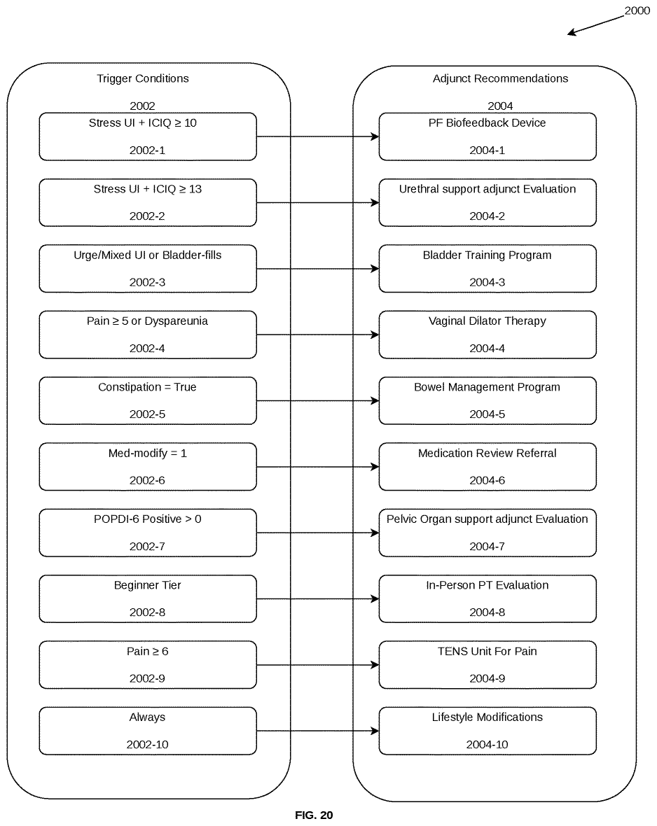

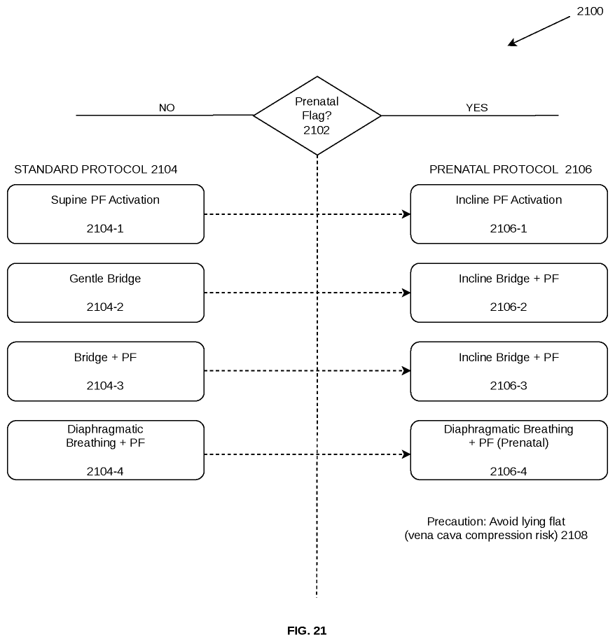

FIG. 20 illustrates a block diagram of a rule-based mapping 2000 between trigger conditions and adjunct therapy recommendations in accordance with an exemplary embodiment of the present invention. The figure demonstrates how adjunct interventions are deterministically assigned based on clinical flags, baseline clinical scores, and other patient-specific indicators. This figure illustrates the implementation of the adjunct sub-module logic, in which predefined trigger conditions 2002 activate corresponding adjunct therapy recommendations 2004 to complement the individualized pelvic floor physical therapy care plan.

As shown, the set of trigger conditions 2002 is evaluated against patient-specific clinical data and derived indicators. Each trigger condition 2002 corresponds to a predefined rule that, when satisfied, results in the assignment of a specific adjunct recommendation 2004. For example, a trigger condition 2002-1 corresponding to stress urinary incontinence with an ICIQ score greater than or equal to a first threshold (e.g., ≥10) results in assignment of a pelvic floor biofeedback device recommendation 2004-1. Similarly, a trigger condition 2002-2 corresponding to stress urinary incontinence with a higher ICIQ threshold (e.g., ≥13) results in the assignment of a urethral support adjunct evaluation 2004-2, configured to address urethral stability and leakage control based on increased symptom severity.

Additional trigger conditions incorporate multi-domain clinical indicators. For instance, a trigger condition 2002-3 corresponding to urge or mixed urinary incontinence or bladder filling symptoms results in assignment of a bladder training program 2004-3. A trigger condition 2002-4 corresponding to pain severity above a defined threshold (e.g., pain≥5) or presence of dyspareunia results in assignment of vaginal dilator therapy 2004-4. A trigger condition 2002-5 indicating constipation results in assignment of a bowel management program 2004-5, addressing bowel-related contributors to pelvic floor dysfunction.

Further, medication-related and diagnostic indicators are incorporated into the rule set. A trigger condition 2002-6, corresponding to a medication modification flag, results in the assignment of a medication review referral 2004-6. A trigger condition 2002-7 corresponding to a positive prolapse-related indicator (e.g., POPDI-6 score greater than zero) results in assignment of a pelvic organ support adjunct evaluation 2004-7, configured to address pelvic organ positioning and prolapse-related symptoms. In certain implementations, the urethral support adjunct evaluation 2004-2 and the pelvic organ support adjunct evaluation 2004-7 correspond to distinct anatomical targets, evaluation protocols, and therapeutic objectives, despite both relating to support-based interventions. In such implementations, the adjunct evaluation may include device-based assessment, including pessary fitting, or other clinically appropriate support strategies selected in accordance with the identified functional objective.

The mapping also incorporates assignment-dependent and severity-dependent conditions. For example, a trigger condition 2002-8, corresponding to assignment to a beginner tier, results in a recommendation for an in-person physical therapy evaluation 2004-8, thereby introducing additional clinician oversight for higher-severity cases. Similarly, a trigger condition 2002-9 corresponding to pain exceeding a higher threshold (e.g., pain≥6) results in assignment of a transcutaneous electrical nerve stimulation (TENS) unit for pain management 2004-9. A default or always-applied trigger condition 2002-10 results in assignment of lifestyle modifications 2004-10, ensuring that baseline behavioral recommendations are consistently included in the care plan.

In this exemplary scenario, FIG. 20 demonstrates how deterministic conditional logic maps clinical findings and assignment outputs to adjunct therapy recommendations in a structured and reproducible manner. Each trigger condition is evaluated independently or in combination, allowing multiple adjunct recommendations to be assigned concurrently based on the patient's clinical profile. Each of the adjunct recommendations is defined in terms of a functional therapeutic objective and associated rule conditions; the mapping preserves traceability between input data, applied rules, and generated outputs. This approach ensures that adjunct therapies are systematically integrated into the overall care plan, enhancing therapeutic effectiveness while maintaining transparency and traceability of decision-making.

FIG. 21 illustrates a block diagram of a prenatal protocol modification workflow 2100 in accordance with an exemplary embodiment of the present invention. This workflow 2100 demonstrates conditional substitution of therapeutic actions based on the detection of a predefined condition, specifically a prenatal flag. This figure illustrates an implementation of the protocol modification module, in which therapeutic actions are automatically replaced with alternative actions while preserving associated parameter values, thereby ensuring patient safety and clinical appropriateness without altering the structural integrity of the care plan.

As shown, the workflow 2100 begins with the evaluation of a prenatal flag 2102, which serves as a condition detection mechanism. The prenatal flag 2102 determines whether a patient is subject to prenatal considerations that require modification of standard therapeutic exercises. When the prenatal flag 2102 is not present, the method proceeds with a standard protocol 2104 comprising a sequence of therapeutic actions. These actions include, for example, supine pelvic floor activation 2104-1, gentle bridge exercises 2104-2, bridge exercises combined with pelvic floor activation 2104-3, and diaphragmatic breathing combined with pelvic floor activation 2104-4. These exercises are representative of baseline therapeutic actions included in an individualized pelvic floor physical therapy care plan under standard conditions.

When the prenatal flag 2102 is present, the workflow 2100 transitions to a prenatal protocol 2106, wherein each standard therapeutic action is systematically substituted with a corresponding prenatal-safe alternative. The substitutions are performed on a one-to-one basis, ensuring that the structure and sequence of the care plan remain consistent while modifying only the execution context of the exercises. For example, the supine pelvic floor activation 2104-1 is substituted with incline pelvic floor activation 2106-1, thereby avoiding a supine position. Similarly, gentle bridge exercises 2104-2 are replaced by incline bridge exercises combined with pelvic floor activation 2106-2, and bridge exercises combined with pelvic floor activation 2104-3 are replaced by incline bridge exercises combined with pelvic floor activation 2106-3. Diaphragmatic breathing combined with pelvic floor activation 2104-4 is substituted with a prenatal-adapted diaphragmatic breathing exercise combined with pelvic floor activation 2106-4.

These substitutions are performed while preserving associated parameter values, such as repetition counts, hold durations, relaxation durations, session frequency, and progression criteria, thereby maintaining consistency in therapeutic intensity and progression. The substitution process ensures that the care plan remains aligned with the patient's clinical profile while adapting to specific physiological constraints. Additionally, the prenatal protocol 2106 may include precautionary guidance 2108, such as avoiding lying flat to reduce the risk of vena cava compression. This precautionary guidance 2108 is incorporated into the modified care plan to provide contextual safety instructions alongside the substituted exercises.

In this exemplary scenario, FIG. 21 illustrates how conditional logic dynamically modifies therapeutic protocols based on patient-specific conditions, without requiring manual reconfiguration of the entire care plan. The structured mapping between standard protocol 2104 actions and the alternatives in prenatal protocol 2106 ensures that modifications are deterministic, transparent, and reproducible. This approach enables the care plan to accommodate special populations, such as prenatal patients, while preserving the overall therapeutic framework and parameterization defined during care plan generation.

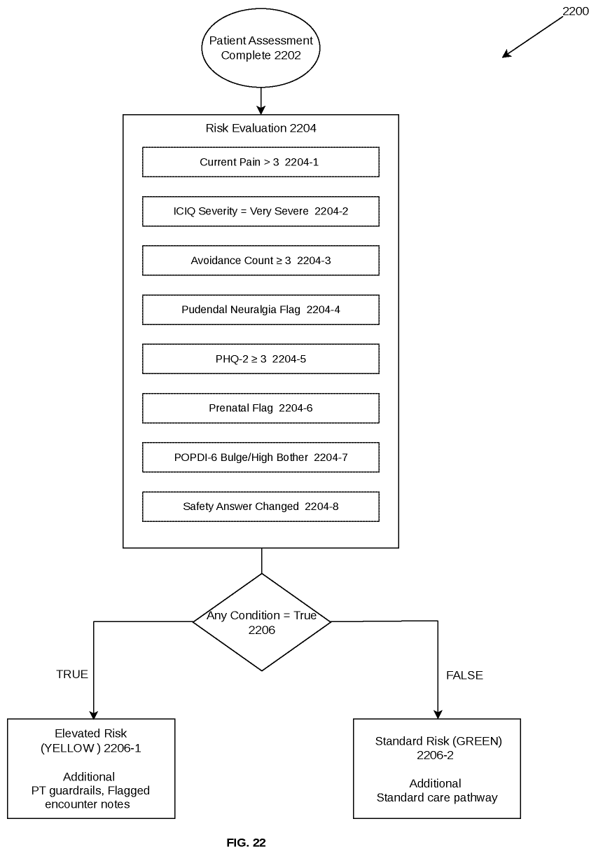

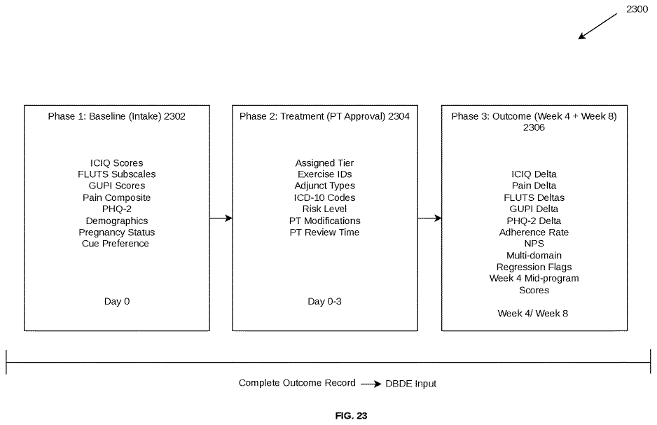

FIG. 22 illustrates a block diagram of a risk evaluation workflow 2200 in accordance with an exemplary embodiment of the present invention. This workflow 2200 determines a patient risk classification based on a set of predefined clinical conditions, consistent with the risk determination module. This figure depicts a deterministic, rule-based evaluation process in which multiple clinical indicators are assessed to classify a patient into a risk category, such as standard or elevated risk, thereby influencing subsequent care plan generation and clinical oversight.

As shown, the workflow 2200 begins after completion of a patient assessment 2202, which includes the collection and processing of patient-specific clinical data and computation of baseline clinical scores and clinical flags. The workflow 2200 proceeds to a risk evaluation stage 2204, where a plurality of predefined conditions are evaluated. These conditions represent combinations of baseline clinical scores, clinical flags, and other derived indicators associated with patient safety and treatment complexity.

The evaluated conditions include, for example, a current pain level exceeding a predefined threshold 2204-1, such as pain greater than three, indicating active symptom severity; an incontinence severity classification corresponding to a very severe category 2204-2 based on ICIQ score ranges; an avoidance count exceeding a predefined threshold 2204-3, reflecting behavioral or functional limitations; and the presence of a pudendal neuralgia flag 2204-4, indicating a specific clinical condition requiring additional attention. Additional conditions include a mental health screening result PHQ-2 exceeding a threshold 2204-5, such as the PHQ-2 score greater than or equal to three, indicating potential depression indicators; detection of a prenatal flag 2204-6, requiring protocol modification considerations; and a prolapse-related indicator POPDI-6 2204-7, such as a POPDI-6 score indicating bulge symptoms or high bother. Further, a safety-related condition 2204-8 may be evaluated, such as the detection of changes in safety responses, which may indicate emerging risks or adverse events.

Each of these conditions is evaluated independently within the risk evaluation stage 2204, and the results are collectively assessed using a logical aggregation rule. In particular, the workflow 2200 applies an “any condition true 2206” logic, where satisfaction of any one or more of the evaluated conditions results in classification of the patient as elevated risk. When one or more conditions evaluate to true, the patient is classified into a yellow risk category 2206-1, corresponding to elevated risk. This classification may trigger additional clinical safeguards, including enhanced physical therapy guardrails, increased monitoring, and the generation of flagged encounter notes requiring clinician review.

Conversely, if none of the evaluated conditions are satisfied, the patient is classified into a green risk category 2206-2, corresponding to standard risk. In this case, the patient proceeds along a standard care pathway without additional risk-based interventions. The standard risk classification indicates that the patient's clinical profile does not meet predefined thresholds requiring elevated oversight.

In this exemplary scenario, FIG. 22 demonstrates how multiple clinical indicators are integrated into a unified risk classification framework using deterministic logic. The use of predefined thresholds and logical aggregation ensures that risk determination is transparent, reproducible, and aligned with clinical safety considerations. The resulting risk classification directly informs downstream processes, including care plan generation, clinical review requirements, and protocol modifications, thereby enabling adaptive and risk-aware management of pelvic floor physical therapy care.

FIG. 23 illustrates a block diagram of a longitudinal data lifecycle workflow 2300 in accordance with an exemplary embodiment of the present invention. The longitudinal data lifecycle workflow 2300 is configured to generate, execute, and evaluate a personalized pelvic floor physical therapy care plan across multiple phases. This figure represents a time-sequenced progression of data collection, clinical decision-making, and outcome evaluation, consistent with the intake, treatment, outcome collection, and feedback processes.

As shown, the workflow 2300 is organized into three primary phases: a baseline intake phase 2302, a treatment phase 2304, and an outcome phase 2306, each corresponding to a distinct stage in the care plan lifecycle. The process begins with phase 1: baseline intake 2302, which occurs at an initial time point, such as day 0. During this phase, patient-specific clinical data are collected, including validated clinical assessment instrument scores (e.g., ICIQ, FLUTS subscales, GUPI, pain composite values) and mental health screening results (e.g., PHQ-2). Additional data elements may include demographic information, pregnancy status, and patient cue preferences, which are used to personalize therapy instructions. A clinical logic version identifier 2302 is embedded at this stage, ensuring that the rules, scoring functions, and thresholds applied during care plan generation are version-controlled and traceable.

Following the baseline intake 2302, the workflow proceeds to phase 2: treatment 2304, which includes clinician review and approval of the generated care plan. During this treatment phase 2304, key treatment parameters are established and recorded, including the assignment of exercise tiers, the specification of exercise identifiers, the recommendations for adjunct therapy, and the assignment of diagnosis codes. A risk level determined during earlier processing is also included. Additionally, clinician interaction data may be captured, including modifications to the care plan and the duration of clinician review. This treatment phase 2304 typically occurs within a short interval following intake, such as day 0 to day 3, and represents the transition from plan generation to active treatment execution.

The workflow 2300 then progresses to phase 3: outcome 2306, which captures longitudinal treatment outcome data over a defined period, such as between week 4 and week 8. During this outcome phase 2306, repeated clinical assessment results are collected, including deltas for ICIQ scores, pain scores, FLUTS subscales, GUPI scores, and PHQ-2 scores, reflecting changes relative to baseline. Additional outcome metrics include the adherence rate, which may be computed using a rolling-window analysis of patient-reported adherence data, and patient satisfaction indicators, such as the net promoter score (NPS). The outcome phase 2306 also includes the identification of multi-domain regression flags based on worsening thresholds across clinical domains, as well as mid-program checkpoint scores collected at an intermediate time point, such as week 4. These data elements collectively represent longitudinal treatment outcome data.

All data generated across the three phases are consolidated into a complete outcome record 2308, which serves as input to downstream analytical processes, including biomarker generation and feedback-driven updates. The outcome record 2308 maintains linkage between baseline inputs, treatment parameters, and outcome measures, thereby supporting auditable traceability and enabling statistical analysis of treatment effectiveness.

In this exemplary scenario, FIG. 23 demonstrates how patient data evolves from initial intake through treatment execution to outcome evaluation, forming a continuous data lifecycle. For example, baseline severity scores collected during phase 1 (baseline intake 2302) may influence exercise tier and adjunct recommendations in phase 2 (treatment 2304), while outcome deltas observed in phase 3 (outcome 2306) may inform reassignment of therapy parameters in subsequent iterations. The inclusion of a clinical logic version identifier ensures that all decisions remain traceable to the specific rules in effect at the time of processing. This structured, phase-based longitudinal data lifecycle workflow 2300 enables comprehensive monitoring, evaluation, and refinement of personalized pelvic floor physical therapy care in a consistent and data-driven manner.

FIG. 24 illustrates a block diagram of an analytical workflow 2400 for generating digital biomarkers in accordance with an exemplary embodiment of the present invention. This workflow 2400 demonstrates how to generate digital biomarkers from aggregated outcome data, consistent with the biomarker generation module and feedback processes. This figure depicts a structured sequence of data filtering, cohort construction, statistical evaluation, and signal classification, enabling identification of statistically significant treatment-response relationships.

As shown, the workflow 2400 begins with a dataset of completed outcome records 2402, which are filtered by a clinical logic version identifier to ensure consistency in the scoring rules, assignment thresholds, and care plan parameters applied. By restricting analysis to records generated under a common clinical logic version, the workflow 2400 ensures comparability and validity of subsequent statistical analysis. The filtered dataset from the complete outcome records dataset 2402 is then subjected to a minimum sample check 2404, wherein a predefined threshold (for example, N≥30) is evaluated to determine whether sufficient data exist to support a statistically meaningful analysis. If the minimum sample requirement is not met, the process may be deferred or flagged for insufficient data.

Upon satisfying the sample requirement, the workflow 2400 proceeds to biomarker candidate selection 2406, where a plurality of candidate variables or features (e.g., BM001-BM005) are identified for evaluation. These candidates may include combinations of baseline clinical scores, behavioral attributes, therapy parameters, or adherence-related variables. Following biomarker candidate selection 2406, cohort separation 2408 is performed to partition the dataset into comparison groups (e.g., a match group and a comparison group), with a minimum of records per cohort (e.g., N≥5 per group). This partitioning enables comparative statistical analysis of treatment outcomes across distinct patient subgroups.

The workflow 2400 then advances to outcome extraction 2410, wherein outcome functions are applied to each record to derive relevant outcome measures, such as symptom-improvement deltas, adherence-adjusted outcomes, or functional-recovery indicators. These extracted outcomes are then subjected to statistical analysis 2412, which includes computation of descriptive statistics, effect size using Cohen's d, and statistical significance testing using Welch's t-test. The use of these statistical measures enables quantification of both the magnitude and reliability of observed differences between cohorts.

Based on the results of statistical analysis 2412, the workflow 2400 performs signal classification 2414, categorizing each evaluated biomarker candidate into predefined categories. In certain scenarios, the predefined categories include a strong signal (2414-1), an emerging signal (2414-2), and an insufficient signal (2414-3). The strong signal 2414-1 may be identified when statistical significance meets a threshold, such as p<0.05, and the effect size exceeds a threshold, such as |d|>0.5. The emerging signal 2414-2 may be identified at intermediate thresholds, such as p<0.10 and |d|>0.3, indicating potential but less-definitive relationships. Conversely, the insufficient signal 2414-3 may be assigned when the candidate does not meet predefined statistical thresholds. These classifications provide a structured mechanism for prioritizing clinically relevant biomarkers.

In this exemplary scenario, FIG. 24 demonstrates how aggregated outcome data is transformed into actionable digital biomarkers via a deterministic, statistically grounded pipeline. For instance, a biomarker candidate linking high adherence rates with improved pain outcomes may be classified as a strong signal if it meets both effect size and significance thresholds, thereby informing future updates to therapy parameters. The resulting classified signals may be fed into downstream processes, such as updating scoring rules, assignment logic, or care plan parameters, thereby enabling continuous refinement of personalized pelvic floor physical therapy care based on empirical evidence.

FIG. 25 illustrates a block diagram of a biomarker candidate architecture 2500 in accordance with an exemplary embodiment of the present invention. The biomarker candidate architecture 2500 defines, organizes, and evaluates candidate digital biomarkers used in statistical analysis and feedback-driven refinement of pelvic floor physical therapy care plans. The architecture 2500 is associated with the biomarker generation module and is configured to support downstream updating performed by the feedback module. This figure provides a structured representation of how candidate biomarkers are specified in terms of hypotheses, matching criteria, outcome variables, and expected directional effects, consistent with the biomarker generation and statistical analysis processes.

As shown, the architecture 2500 is organized in a tabular format, where each row corresponds to a distinct biomarker candidate identified by a unique identifier (ID), such as BM001 through BM005. Each biomarker candidate includes a hypothesis field defining a clinical or behavioral relationship being evaluated, a match criteria field specifying conditions used to partition patient records into cohorts, an outcome field identifying a measured outcome variable, a direction field indicating an expected directional effect on the outcome variable, and a reference field configured for traceability or linkage to supporting data.

In an illustrative case, a first biomarker candidate (BM001) is defined as a non-response predictor, with match criteria including a combination of elevated GUPI score (e.g.,≥20), avoidance count (e.g., ≥3), and a mental health screening indicator such as PHQ-2 score (e.g., ≥3). The associated outcome is defined as a change in the ICIQ score (ICIQ Δ), and the expected direction is negative, indicating that patients meeting these criteria are hypothesized to exhibit reduced improvement or to worsen. A second biomarker candidate (BM002) is directed to early detection of a specific condition, such as pudendal involvement, with match criteria including elevated FLUTS scores, pain thresholds, and behavioral indicators such as prolonged sitting. This candidate also evaluates ICIQ Δ with a negative directional expectation.

A third biomarker candidate (BM003) serves as a dropout predictor, incorporating match criteria such as avoidance behaviors, mental health indicators, default cue usage, and the absence of prior treatment. This candidate similarly evaluates ICIQ Δ with a negative direction, reflecting an expected association with reduced adherence or suboptimal outcomes. A fourth biomarker candidate (BM004) represents a cue-response interaction by comparing outcomes across different cueing strategies (e.g., breath-based versus biologic cueing) within a defined clinical subgroup (e.g., GUPI≥15), with a positive expected direction. A fifth biomarker candidate (BM005) represents a tier boundary signal, examining outcome differences across predefined score ranges corresponding to tier assignment thresholds, with an exploratory direction indicating that the relationship is evaluated without a predefined directional assumption.

In this exemplary scenario, FIG. 25 demonstrates how biomarker candidates are systematically defined before statistical evaluation, enabling reproducible and interpretable analysis of treatment-response relationships. Each candidate is applied to partition aggregated outcome records into cohorts based on the specified match criteria, after which statistical analysis, including effect size computation and significance testing, is performed. The structured format ensures that each biomarker candidate is explicitly linked to clinical variables, behavioral attributes, and therapy parameters, thereby supporting transparent evaluation and integration into the biomarker generation module. Resulting validated biomarkers are propagated to the feedback module to update deterministic scoring functions, assignment thresholds, therapy personalization strategies, and risk classification logic, thereby enabling continuous refinement of personalized pelvic floor physical therapy care.

FIG. 26 illustrates a block diagram of a closed-loop evidence generation architecture 2600 in accordance with an exemplary embodiment of the present invention. The architecture 2600 demonstrates how deterministic clinical decision logic is refined through longitudinal outcome data and statistical biomarker analysis while preserving a rule-based care plan generation framework. As shown, the closed-loop evidence generation architecture 2600 includes a deterministic clinical decision engine (DCDE) 2602, a patient execution stage 2604, outcome records 2606, a digital biomarker decision engine (DBDE) 2608, and a feedback path 2610 that collectively define a closed-loop refinement process. The deterministic clinical decision engine 2602 corresponds to processing components, including the deterministic scoring module, assignment module, risk determination module, and care plan generation module.

As shown, the process begins with the deterministic clinical decision engine 2602, which generates individualized care plans based on deterministic scoring outputs, predefined clinical thresholds, and assignment logic derived from patient-specific data. The deterministic clinical decision engine 2602 incorporates validated assessment instruments, clinical flags, severity mappings, risk classifications, and protocol selection rules to produce a structured therapeutic plan. The generated care plan is then provided to the patient execution stage 2604, where prescribed exercise programs and associated therapeutic actions are performed in accordance with the generated plan.

Based on the execution of the patient-specific therapy program, 2606 outcome records are generated and maintained, including baseline data, treatment data, and longitudinal outcome data, each associated with a clinical logic version identifier. The outcome records 2606, preserving an auditable linkage between patient-specific input data, computed clinical scores, applied decision rules, generated care plan parameters, and observed patient responses over time. Changes in symptoms, adherence patterns, reassessment results, and adverse event data are captured within the outcome records 2606 to enable longitudinal evaluation relative to the original deterministic care plan.

The workflow proceeds to the digital biomarker decision engine 2608, which analyzes the outcome records 2606 using statistical techniques to identify treatment-response patterns. In one or more implementations, the digital biomarker decision engine 2608 performs descriptive statistical analysis, effect size computation using Cohen's d, and statistical significance testing using Welch's t-test to evaluate whether observed changes are clinically meaningful and statistically supported. Based on the analysis, the digital biomarker decision engine 2608 identifies statistically significant patterns that are eligible for incorporation into clinical logic refinement. The feedback path 2610 represents transfer of such validated patterns to a feedback module configured to update scoring rules, threshold values, tier assignment criteria, adjunct therapy mappings, or risk classification logic within the deterministic clinical decision engine 2602.

The closed-loop evidence generation architecture 2600, therefore, enables controlled refinement of deterministic clinical logic while preserving reproducibility, transparency, and auditability. The statistical outputs generated by the digital biomarker decision engine 2608 do not directly replace deterministic decision logic; rather, they inform updates to predefined rules and parameters that govern subsequent care plan generation. Threshold conditions may be applied at multiple stages to determine whether refinement is warranted, including conditions related to symptom improvement, adherence consistency, severity escalation, or the occurrence of adverse events. This ensures that modifications to clinical logic are evidence-driven and systematically incorporated.

In this exemplary scenario, a patient executes a prescribed exercise program generated by the deterministic clinical decision engine 2602, outcome data are collected into the outcome records 2606, the digital biomarker decision engine 2608 identifies a statistically significant treatment-response pattern through comparative analysis, and the validated pattern is transmitted via the feedback path 2610 to update deterministic scoring parameters or assignment thresholds. Subsequent care plans generated for similar patient profiles reflect the refined logic, thereby enabling continuous, evidence-based improvement of personalized pelvic floor physical therapy care.

FIG. 27 illustrates a block diagram of a clinical review flag architecture 2700 in accordance with an exemplary embodiment of the present invention. The architecture 2700 defines a deterministic, rule-based flag-generation framework that separates always-review flags from triggered flags to support automated clinical oversight within a pelvic floor physical therapy care plan system. The architecture 2700 is implemented in association with a clinical review module, including a guardrail sub-module and a flag sub-module. As shown, the clinical review flag architecture 2700 includes an always-review flag set 2702, a triggered flag set 2704, and a phase-based automation logic 2706 that collectively determine whether a patient care plan requires clinician review at plan creation or during subsequent monitoring intervals.

As shown, the always-review flag set 2702 is generated during care plan generation and includes flags that require clinician review before plan finalization. The always-review flag set 2702 is deterministically derived from validated clinical inputs using predefined threshold conditions. In the illustrated architecture 2700, the always-review flag set 2702 includes a high severity ICIQ flag 2702-1 corresponding to ICIQ greater than or equal to 13, a depression risk flag 2702-2 corresponding to PHQ-2 greater than or equal to 3, a prenatal flag 2702-3 corresponding to a pregnancy condition, a pudendal suspected flag 2702-4 indicating suspected pudendal neuralgia, a prolapse review flag 2702-5 corresponding to a positive POPDI-6 result, a safety answer changed flag 2702-6 indicating a change in safety responses, and a no-incontinence flag 2702-7 corresponding to ICIQ equal to zero. Each flag is generated based on a predefined rule and configured to trigger a mandatory clinical review before the care plan is activated.

The triggered flag set 2704 is generated during post-treatment monitoring and reflects clinically relevant changes occurring after therapy initiation. The triggered flag set 2704 is derived from longitudinal outcome data captured by the outcome collection module and evaluated using deterministic threshold conditions and regression detection logic. As shown, the triggered flag set 2704 includes an adherence concern flag 2704-1 corresponding to a rolling adherence rate below a predefined threshold (e.g., 14-day adherence rate less than 50%), a clinical regression flag 2704-2 corresponding to a worsened ICIQ result relative to baseline, a pain worsened flag 2704-3 corresponding to a pain increase of at least 2 points, a bowel worsened flag 2704-4 indicating worsening bowel symptoms, a FLUTS worsened flag 2704-5 corresponding to a FLUTS increase exceeding a predefined threshold, a GUPI-U worsened flag 2704-6 corresponding to a GUPI-U increase exceeding a predefined threshold, a prolapse worsened flag 2704-7 indicating worsening prolapse symptoms, and an adverse event flag 2704-8 corresponding to an adverse event severity exceeding a predefined escalation threshold. These triggered flags enable detection of deterioration, non-adherence, or adverse outcomes during care plan execution.

The phase-based automation logic 2706 governs the application of the flag sets 2702 and 2704 within the clinical workflow. In the first operational phase, all care plans are subject to clinician review, such that the presence of any always-review flag 2702 routes the plan for manual evaluation before activation and may prevent plan finalization until the review conditions are satisfied. In a subsequent operational phase, care plans not associated with always-review flags may proceed without initial review; however, detection of any triggered flag 2704 during longitudinal monitoring results in the generation of a review requirement, escalation event, or modification workflow. This phased control structure ensures consistent application of deterministic review rules while enabling dynamic escalation based on observed patient response.

The architecture 2700, therefore, provides a structured guardrail mechanism linking baseline assignment logic to longitudinal monitoring and escalation control. Each clinical condition is evaluated against predefined threshold criteria or change-based rules, and the resulting flags are used to determine whether clinician intervention is required at plan initiation or during follow-up intervals. By separating always-review conditions from triggered conditions, the architecture supports transparent governance of safety-sensitive cases while enabling automated detection of regression, non-adherence, or adverse events within the personalized care plan environment.

In this exemplary scenario, a patient with elevated ICIQ and PHQ-2 scores is assigned an always-review flag at plan creation, requiring clinician review before the care plan is activated. In contrast, a patient initially classified as low risk may later generate an adherence concern flag and a pain worsened flag during longitudinal monitoring, resulting in escalation of the care plan for clinician review and potential modification based on updated clinical conditions.

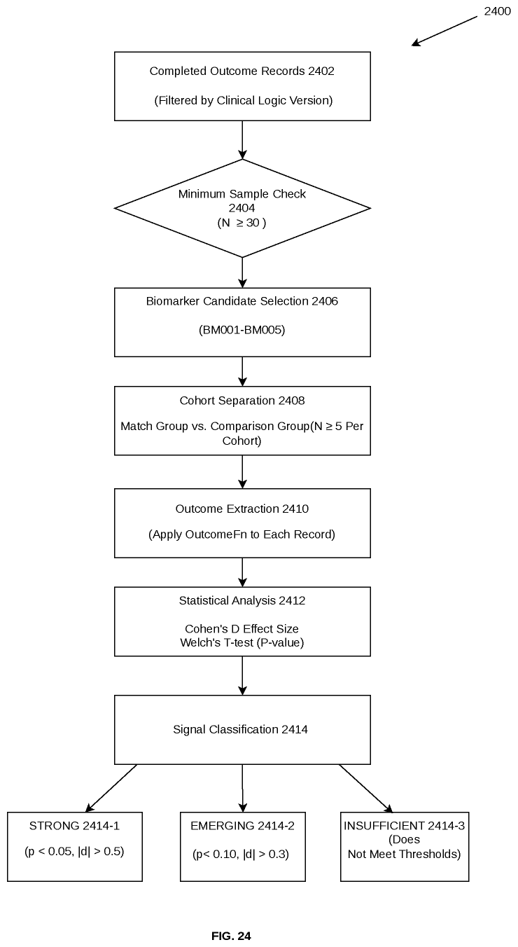

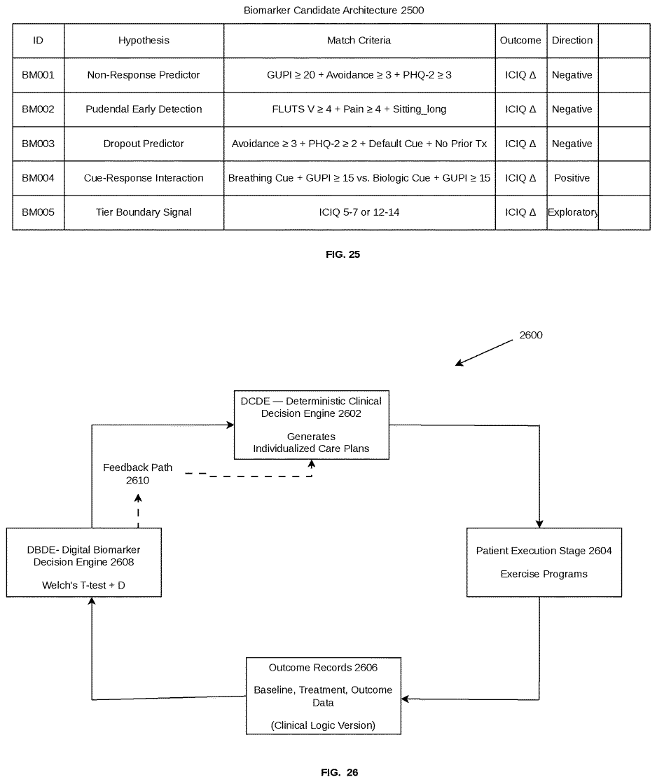

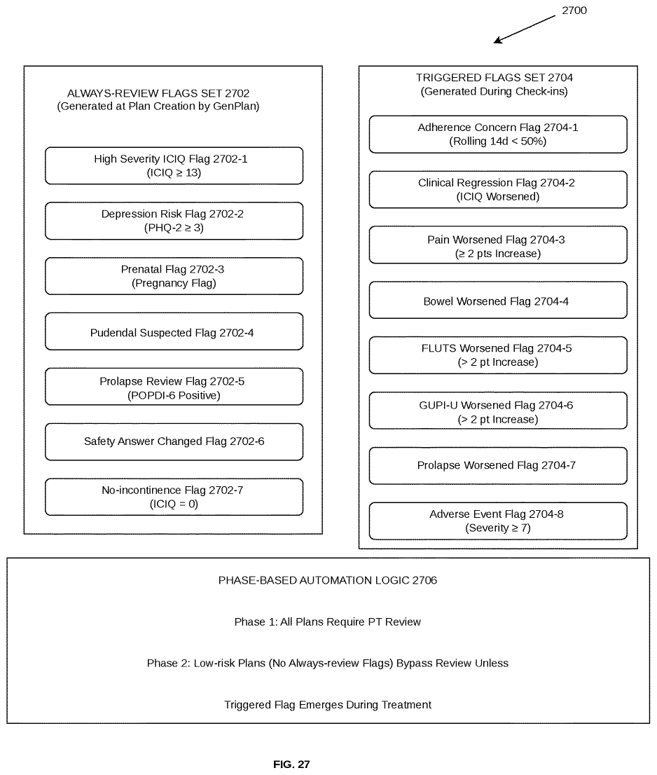

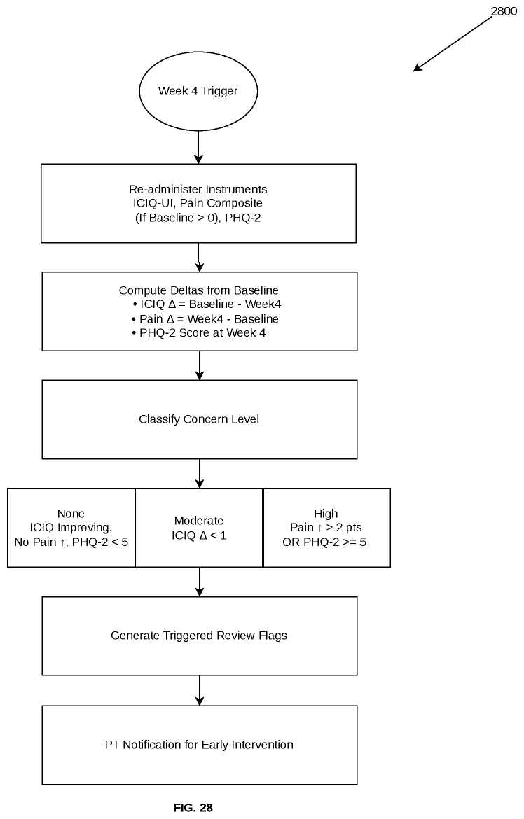

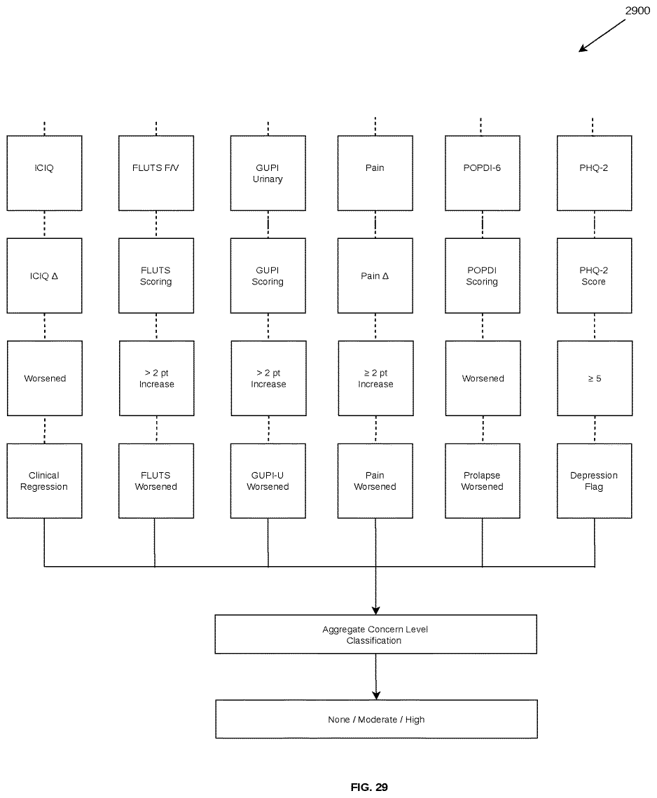

FIG. 28 illustrates a longitudinal reassessment workflow 2800 in accordance with the present invention. The workflow 2800 defines a deterministic mid-program clinical checkpoint for reevaluating patient outcomes at a scheduled follow-up interval, such as week 4, in order to convert baseline and interim clinical data into triggered review logic. The workflow 2800 is implemented in association with the outcome collection module, regression detection module, and clinical review module. As shown, the longitudinal reassessment workflow 2800 includes a week 4 trigger, a re-administer instruments stage, a compute deltas from baseline stage, a classify concern level stage, concern-level branches, a generate triggered review flags stage, and a PT notification for early intervention stage. The workflow 2800 further includes an audit record and a transformation note indicating conversion of the outcome record from a two-phase model to a three-phase longitudinal model.

As shown, the process begins with the week 4 trigger, which initiates reassessment after a predetermined interval has elapsed from baseline care plan execution. The workflow 2800 proceeds to the re-administer instruments stage, where validated clinical instruments, including ICIQ-UI SF, pain composite, and PHQ-2, are re-administered. Selection of instruments at this stage may be conditionally determined based on baseline values or qualifying criteria, thereby ensuring consistent longitudinal measurement across relevant domains. The re-administration stage captures updated patient responses using the same validated instruments used at baseline, enabling direct and reproducible comparison between baseline and follow-up values.

The workflow 2800 proceeds to the compute deltas from baseline stage, where follow-up values are compared to baseline values to calculate domain-specific change metrics. In the illustrated workflow 2800, the delta computations include an ICIQ delta, a pain delta, and a PHQ-2 value at week 4, thereby producing structured outputs that quantify clinical change over time. Each delta is computed using predefined formulas, such as baseline minus follow-up for ICIQ and follow-up minus baseline for pain, so that improvement or worsening is consistently represented across patients. These domain-specific delta values are aligned with regression detection logic and provide standardized inputs for subsequent classification.

Based on the computed delta values, the workflow 2800 advances to the classify concern level stage, which assigns the patient to a deterministic concern category based on predefined threshold conditions. When the patient satisfies improvement or stability criteria, the patient is classified in a no concern category, corresponding to improved ICIQ, no increase in pain, and PHQ-2 below a predefined threshold (e.g., less than 5). When the patient exhibits intermediate change not meeting escalation thresholds, the patient is classified in a moderate concern category, corresponding to limited improvement (e.g., ICIQ delta less than a predefined improvement threshold). When the patient satisfies worsening criteria, the patient is classified in a high concern category, corresponding to pain increase exceeding a predefined threshold (e.g., ≥2 points) or PHQ-2 meeting or exceeding a predefined escalation threshold. This threshold-based classification ensures that concern level determination is transparent, reproducible, and clinically interpretable.

Following classification, the workflow 2800 proceeds to the generate triggered review flags stage, where flags are deterministically generated for patients whose reassessment results satisfy escalation conditions. The triggered review flags may be derived from moderate or high concern categories and are used by the clinical review module to determine whether clinician intervention is required. The workflow 2800 then proceeds to the PT notification stage, which initiates an early intervention process by notifying a clinician or therapy workflow to review or modify the care plan. The audit record stores reassessment data, computed deltas, classification results, and generated flags in association with a clinical logic version identifier, thereby maintaining an auditable linkage between inputs, decisions, and outcomes.

The transformation note indicates that the reassessment workflow 2800 converts an outcome record from a two-phase longitudinal model to a three-phase longitudinal model, thereby extending analysis to include baseline, treatment, and follow-up phases. This transformation enables iterative refinement of care plan logic based on repeated measurements over time rather than a single post-treatment comparison. In this exemplary scenario, a patient returning at week 4 is re-evaluated using validated instruments, domain-specific delta values are computed relative to baseline, a concern level is deterministically assigned based on predefined thresholds, and when escalation criteria are satisfied, a triggered review flag is generated, and a clinician notification is issued to enable timely intervention.

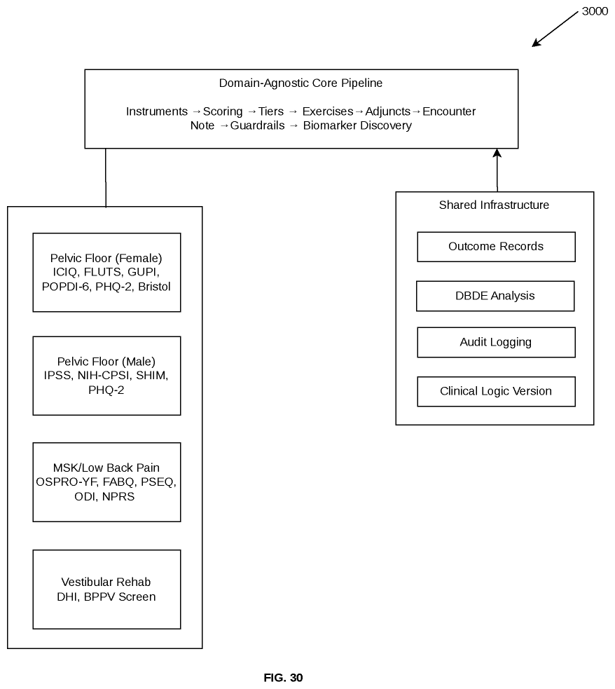

FIG. 29 illustrates a multidomain concern aggregation workflow 2900 in accordance with the present invention. The workflow 2900 implements functionality corresponding to the multi-domain regression detection module by evaluating multiple clinical domains independently and aggregating the results into an overall concern classification. As shown, the multidomain concern aggregation workflow 2900 includes an ICIQ branch, a FLUTS F/V branch 2904, a GUPI urinary branch, a pain branch, a POPDI-6 branch, and a PHQ-2 branch. Each branch is configured to process domain-specific inputs and generate corresponding domain-specific deltas and flags that contribute to downstream concern evaluation.

As shown, the ICIQ branch receives an ICIQ score and computes an ICIQ delta representing a difference between baseline and follow-up values. The computed delta is evaluated against a predefined worsening threshold, and when the threshold condition is satisfied, a clinical regression flag is generated. Similarly, the FLUTS F/V branch evaluates FLUTS scoring and generates a FLUTS worsened flag when the score change exceeds a predefined threshold, such as greater than two points. The GUPI urinary branch evaluates GUPI scoring and generates a GUPI-U worsened flag when the score increases beyond a corresponding threshold value. Each of these branches operates independently using deterministic threshold comparisons, thereby preserving domain-specific sensitivity.

The workflow 2900 further includes the pain branch, the POPDI-6 branch, and the PHQ-2 branch. The pain branch 2908 computes a pain delta and generates a pain worsened flag when the increase in pain meets or exceeds a predefined threshold, such as an increase of at least two points. The POPDI-6 branch evaluates POPDI scoring and generates a prolapse worsened flag when worsening is detected according to predefined criteria. The PHQ-2 branch 2912 evaluates the PHQ-2 score and generates a depression flag when the score meets or exceeds a predefined threshold, such as greater than or equal to five. Each condition is evaluated using deterministic rules that correspond to clinically meaningful changes within the respective domain.

The workflow 2900 proceeds to an aggregate concern level classification stage, where the domain-specific flags generated by the branches are combined to determine an overall concern level. In one implementation, the aggregate concern level classification produces classifications including none, moderate, or high based on a rule-based evaluation of the presence, number, and severity of domain-specific flags. The aggregation logic maintains independence of domain-level evaluations while enabling a composite clinical interpretation that reflects multi-domain regression. The resulting concern level may be used to generate triggered review flags, initiate clinician review, or modify care plan parameters.

The multidomain concern aggregation workflow 2900 ensures that worsening in any individual clinical domain is detectable independently of changes in other domains, thereby preventing improvement in one domain from masking deterioration in another. This approach enables comprehensive longitudinal monitoring across multiple validated clinical instruments, including urinary, pain, prolapse, and mental health domains. The aggregated concern level supports downstream processes, including clinical review flag generation, risk reclassification, protocol modification, and feedback-driven refinement of deterministic care plan logic within the closed-loop architecture

In this exemplary scenario, a patient may demonstrate improvement in ICIQ while simultaneously worsening in pain or PHQ-2, causing the workflow 2900 to classify the patient at a higher concern level based on the aggregate effect of the domain-specific flags.

FIG. 30 illustrates a domain-agnostic core pipeline 3000 in accordance with the present invention. The domain-agnostic core pipeline 3000 demonstrates that a common deterministic clinical processing architecture may be applied across multiple clinical domains while preserving consistent implementation of an input module, a deterministic scoring module, an assignment module, a risk determination module, a care plan generation module, an outcome collection module, and a feedback module. As shown, the domain-agnostic core pipeline 3000 includes a shared processing flow and a shared infrastructure configured to support a rule-based framework that operates consistently across distinct therapeutic contexts.

As shown, the shared processing flow defines a standardized sequence of operations beginning with receipt of instrument responses, followed by deterministic scoring, classification into severity ranges, assignment of tiers and therapy profiles, generation of parameterized exercises and adjunct therapies, encounter documentation generation, application of clinical guardrails, and execution of biomarker discovery processes. Each stage within the shared processing flow corresponds to one or more modules of the processing module, including the deterministic scoring module, assignment module, risk determination module, care plan generation module, clinical review module, and biomarker generation module. This structure ensures that validated patient-reported data are processed through a consistent deterministic logic pipeline to produce individualized care plans and associated outputs.

The left-side domain-specific grouping illustrates that the domain-agnostic core pipeline 3000 accepts different sets of validated clinical assessment instruments depending on the clinical domain while maintaining the same downstream processing logic. For example, a pelvic floor female group may include ICIQ, FLUTS, GUPI, POPDI-6, PHQ-2, and Bristol measures; a pelvic floor male group may include IPSS, NIH-CPSI, SHIM, and PHQ-2; a musculoskeletal or low back pain group may include OSPRO-YF, FABQ, PSEQ, ODI, and NPRS; and a vestibular rehabilitation group may include DHI and BPPV screening. Each instrument set is mapped to corresponding deterministic scoring functions and severity thresholds within the deterministic scoring module, thereby enabling domain-specific inputs to be normalized into standardized baseline clinical scores and clinical flags without altering the core architecture.

The shared infrastructure provides cross-domain support functions that are common to all clinical use cases and correspond to the memory module and analytical modules of the architecture. In particular, the shared infrastructure includes outcome records configured to store longitudinal treatment data, DBDE analysis configured to perform statistical analysis including descriptive statistics, effect size computation, and significance testing, audit logging configured to maintain auditable linkage between inputs, computed scores, applied rules, generated care plans, and outcomes, and a clinical logic version configured to track the version of deterministic scoring rules, assignment thresholds, and therapy parameters applied. These infrastructure elements enable consistent evidence capture, traceability, and feedback-driven refinement across all supported domains.

The domain-agnostic core pipeline 3000, therefore, enables substitution of domain-specific instrument inputs, scoring mappings, and therapy parameter definitions while preserving a fixed deterministic processing sequence. Thresholds, classification rules, and assignment logic are applied via domain-specific mappings integrated into the deterministic scoring and assignment modules, without modifying the overall workflow structure. This approach supports scalability across multiple therapeutic areas while maintaining reproducibility, auditability, and consistency in care plan generation.

In this exemplary scenario, pelvic floor therapy, male urology, musculoskeletal rehabilitation, and vestibular rehabilitation workflows each utilize the same core pipeline 3000, with differences limited to the selected validated clinical assessment instruments, associated deterministic scoring functions, and corresponding therapy parameter definitions. The resulting outputs, including care plans, outcome records, and biomarker analyses, are generated using the same processing framework, thereby enabling cross-domain application of deterministic clinical logic and longitudinal, evidence-based refinement.

FIG. 30 further demonstrates that the disclosed processing pipeline operates independently of any specific clinical specialty and is adaptable across multiple domains through substitution of domain-specific input instruments, scoring functions, and intervention libraries, while maintaining a consistent deterministic processing architecture. This domain-agnostic configuration enables reuse of the underlying rule-based framework without modification to the core computational logic.

In an illustrative implementation outside the pelvic floor domain, the system is applied to cardiovascular rehabilitation. Patient-specific clinical data includes cardiac risk scores, exercise tolerance measures, and patient-reported symptom data collected using validated cardiovascular assessment instruments. The deterministic scoring module computes severity classifications and risk indicators based on predefined cardiovascular thresholds. The assignment module assigns a rehabilitation tier and therapy profile corresponding to the patient's functional capacity and risk classification. The care plan generation module generates a structured cardiac rehabilitation program including exercise prescriptions, monitoring intervals, and progression criteria. Longitudinal outcome data, including adherence data and functional improvement metrics, are collected by the outcome collection module and utilized by the feedback module to update subsequent care plans. This example demonstrates the applicability of the disclosed deterministic architecture across distinct clinical domains while preserving rule-based operation and controlled feedback refinement.

The foregoing description of embodiments of the present disclosure has been provided for purposes of illustration and description. It is not intended to be exhaustive or to limit the disclosure to the precise forms disclosed. Modifications and variations are possible in light of the above teachings or may be acquired from the practice of the disclosure.

In some embodiments, the methods described herein may be implemented as computer-executable instructions stored on a non-transitory computer-readable medium, including but not limited to memory devices, storage disks, or cloud-based storage systems. Execution of such instructions by one or more processors causes the system to perform the operations described herein.

The system may be implemented in a distributed computing environment in which components are located across multiple computing devices connected via one or more networks. Processing may be performed by central processing units (CPUs), graphics processing units (GPUs), or specialized processing hardware.

The structured clinical data, care plan objects, intervention mappings, and outcome records described herein constitute specific data structures that enable deterministic processing and traceable transformation of input data into clinically actionable outputs. These data structures improve computing systems' ability to store, process, and retrieve clinically relevant information in a consistent and reproducible manner.

1. A system for deterministic generation and adaptive management of a personalized pelvic floor physical therapy care plan, comprising:
an input module to receive patient-specific clinical data, including responses to a plurality of validated clinical assessment instruments;
a deterministic scoring module to compute one or more baseline clinical scores and one or more clinical flags from the patient-specific clinical data using deterministic scoring functions;
an assignment module to assign the patient to at least one exercise tier, at least one therapy profile, and one or more diagnosis codes based on the baseline clinical scores and clinical flags;
a risk determination module to determine a patient risk classification based on the exercise tier, therapy profile, diagnosis codes, baseline clinical scores, and clinical flags;
a care plan generation module to generate an individualized pelvic floor physical therapy care plan based on the patient risk classification;
an outcome collection module to collect longitudinal treatment outcome data generated from execution of the individualized care plan; and
a feedback module to update the baseline clinical scores, clinical flags, exercise tier, therapy profile, diagnosis codes, and patient risk classification based on the longitudinal treatment outcome data for subsequent care plan generation.

2. The system according to claim 1, wherein the plurality of validated clinical assessment instruments includes at least one of an incontinence instrument, a pain instrument, a prolapse instrument, a bowel function instrument, or a mental health screening instrument.

3. The system according to claim 1, wherein the deterministic scoring module computes severity classifications by mapping baseline clinical scores to predefined severity ranges and generating composite scores derived from multiple instrument values.

4. The system according to claim 1, wherein the clinical flags include at least one of constipation indicators, depression indicators, prolapse indicators, or pain severity indicators.

5. The system according to claim 1, wherein the deterministic scoring functions include a composite pain score derived from reuse of a validated instrument pain item as both a component of the validated instrument score and a component of a custom composite score while preserving the original instrument structure.

6. The system according to claim 1, wherein the assignment module assigns the exercise tier based on predefined severity thresholds.

7. The system according to claim 1, wherein the care plan generation module generates parameterized therapeutic exercises including at least one of repetition counts, hold durations, relaxation durations, session frequency, or progression criteria.

8. The system according to claim 1, wherein the assignment module assigns diagnosis codes based on predefined mappings between clinical score ranges and diagnostic classifications.

9. The system according to claim 1, wherein the risk determination module classifies patients into at least standard risk and elevated risk categories based on combinations of pain severity, prolapse indicators, and mental health screening results.

10. The system according to claim 1, wherein the care plan generation module personalizes exercise instructions based on a received patient cue preference, including at least one of a biologic cueing, an imaginative cueing, a breath-based cueing, or a contraction cueing.

11. The system according to claim 1, wherein the system further comprises a documentation module that generates structured SOAP clinical encounter documentation from the individualized care plan.

12. The system according to claim 1, wherein the system further comprises a clinical review module to enforce clinical guardrail checks during plan modifications.

13. The system according to claim 1, wherein the longitudinal outcome data includes at least one of a symptom progression, a treatment adherence data, functional outcome measures, or repeated clinical assessment results.

14. The system according to claim 1, wherein the system further comprises embedding a clinical logic version identifier within generated care plans and outcome records, and updating the version identifier when scoring rules, assignment thresholds, diagnosis mappings, or therapy parameters are modified.

15. The system according to claim 1, wherein the input module further enforces field-level validation with conditional instrument completion requirements to prevent incomplete instrument scoring from influencing deterministic scoring outputs.

16. The system according to claim 1, wherein the assignment module executes conditional logic branches that modify the individualized care plan based on threshold values of the baseline clinical scores or clinical flags.

17. The system according to claim 1, wherein the system further comprises a protocol modification module to automatically substitute one or more therapeutic actions with alternative actions while preserving associated parameter values upon detection of a predefined condition.

18. The system according to claim 1, wherein the feedback module performs statistical analysis comprising descriptive statistics, effect sizes using Cohen's d, and statistical significance using Welch's t-test.

19. The system according to claim 1, wherein the system further comprises a biomarker generation module that partitions patients into subgroups based on combinations of baseline clinical scores, behavioral attributes, and therapy parameters before statistical analysis for biomarker generation.

20. The system according to claim 1, wherein the system maintains an auditable linkage between patient-specific input data, computed clinical scores, applied rules, generated care plan parameters, collected outcome data, and updated decision parameters.

21. The system according to claim 1, wherein the system further comprises a mid-program clinical checkpoint at a predetermined interval that re-administers a subset of baseline clinical instruments, computes deltas relative to baseline, classifies a concern level using deterministic thresholds, and generates triggered review flags for a supervising clinician.

22. The system according to claim 12, wherein the clinical review module enforces two categories of review flags: a) always-review flags deterministically generated at care plan creation based on baseline instrument scores and risk factors, and b) triggered flags dynamically generated during post-treatment monitoring based on longitudinal score changes, adherence patterns, or adverse event reports.

23. The system according to claim 1, wherein the system further comprises a daily adherence logging sub-module to receive patient adherence entries, permit retroactive entries with a backfill marker, compute rolling adherence rates over a defined window, generate adherence concern flags when thresholds are not met, and perform month-level aggregation.

24. The system according to claim 1, wherein the system comprises a structured adverse event capture sub-module with severity-gated escalation.

25. The system according to claim 24, wherein the structured adverse event capture sub-module collects new-symptom indicators, free-text descriptions, and numeric severity scales, elevates concern levels when severity exceeds a predefined threshold, generates adverse event review flags, and incorporates the adverse event data into outcome records for biomarker analysis.

26. The system according to claim 1, wherein the system further comprises a multi-domain regression detection module for a post-treatment checkpoint.

27. The system according to claim 26, wherein the multi-domain regression detection module computes domain-specific deltas independently for a plurality of clinical instruments, applies worsening thresholds for each domain, and generates domain-specific review flags when regression exceeds the respective thresholds.

28. The system according to claim 1, further comprising an agreement analysis module to compute an AI-clinician agreement rate from aggregated outcome records by comparing a number of care plans approved without clinician modifications to a total number of completed care plans, and to present the agreement rate as a regulatory compliance metric.

29. A method for deterministic generation and adaptive management of a personalized pelvic floor physical therapy care plan, comprising:
receiving patient-specific clinical data, including responses to a plurality of validated clinical assessment instruments;
computing one or more baseline clinical scores and one or more clinical flags using deterministic scoring functions;
assigning a patient to at least one exercise tier, at least one therapy profile, and one or more diagnosis codes based on the baseline clinical scores and clinical flags;
determining a patient risk classification based on the exercise tier, therapy profile, diagnosis codes, baseline clinical scores, and clinical flags;
generating an individualized pelvic floor physical therapy care plan based on the patient's risk classification;
collecting longitudinal treatment outcome data generated from execution of the individualized care plan; and
updating the baseline clinical scores, clinical flags, exercise tier, therapy profile, diagnosis codes, and patient risk classification based on the longitudinal treatment outcome data for subsequent care plan generation.

30. A system for deterministic generation and adaptive management of a personalized pelvic floor physical therapy care plan, comprising:
an input module to receive patient-specific clinical data, including responses to a plurality of validated clinical assessment instruments;
a deterministic scoring module to compute one or more baseline clinical scores and one or more clinical flags from the patient-specific clinical data using deterministic scoring functions;
an assignment module to assign a patient to at least one exercise tier, at least one therapy profile, and one or more diagnosis codes based on the baseline clinical scores and clinical flags;
a risk determination module to determine a patient risk classification based on the exercise tier, therapy profile, diagnosis codes, baseline clinical scores, and clinical flags;
a care plan generation module to generate an individualized pelvic floor physical therapy care plan based on the patient's risk classification;
an outcome collection module to collect longitudinal treatment outcome data generated from execution of the individualized care plan;
a biomarker generation module to receive aggregated clinical data, therapy data, and longitudinal treatment outcome data, and generates digital biomarkers by applying statistical analysis to detect statistically significant clinical patterns; and
a feedback module to update the baseline clinical scores, clinical flags, exercise tier, therapy profile, diagnosis codes, and patient risk classification based on the generated digital biomarkers and the longitudinal treatment outcome data for subsequent care plan generation.
